(19) United States
(12) Patent Application Publication
(10) Pub. No.: US 2003/0129223 A1
(43) Pub. Date: Jul. 10, 2003

(54) Targeted multivalent macromolecules

(75) Inventors: Charles Aaron Wartchow, San Francisco, CA (US); Neal Edward DeChene, Morgan Hill, CA (US); John S. Pease, Los Altos, CA (US); Zhimin Shen, Palo Alto, CA (US); Julie Trulson, San Jose, CA (US); Mark David Bednarski, Los Altos, CA (US); S. Narasimhan Danthi, Mountain View, CA (US); Michael Zhang, San Jose, CA (US); Hoyul Steven Choi, San Jose, CA (US)

Correspondence Address: SWANSON & BRATSCHUN L.L.C., 1745 SHEA CENTER DRIVE, SUITE 330, HIGHLANDS RANCH, CO, 80129, US

(73) Assignee: TARGESOME, INC.

(21) Appl. No.: 10/158,777

(22) Filed: May 30, 2002

Related U.S. Application Data
(63) Continuation-in-part of application No. 09/976,254, filed on Oct. 11, 2001.
(60) Provisional application No. 60/239,684, filed on Oct. 11, 2000.
(60) Provisional application No. 60/309,104, filed on Jul. 31, 2001.
(60) Provisional application No. 60/312,435, filed on Aug. 15, 2001.
(60) Provisional application No. 60/294,309, filed on May 30, 2001.

(51) Int. Cl. A61K 39/395; A61K 9/127

(52) U.S. Cl. 424/450; 424/146.1; 424/178.1

(57) ABSTRACT

Targeted therapeutic agents, comprising a linking carrier, a therapeutic entity associated with the linking carrier, and at least one targeting entity are provided, as well as methods for their preparation and use.

### CROSS REFERENCE TO RELATED APPLICATIONS

This application is a continuation-in-part of U.S. patent application Ser. No. 09/976,254, “Targeted Therapeutic Agents,” filed Oct. 11, 2001, which claims the benefit of U.S. Provisional Patent Application Serial No. 60/239,684, “Vascular-Targeted Therapeutic Agents,” filed Oct. 11, 2000. This application also claims the benefit of U.S. Provisional Patent Application Serial No. 60/294,309, “Delivery System for Nucleic Acids,” filed May 30, 2001; U.S. Provisional Patent Application Serial No. 60/309,104, “Synthesis of Multivalent Nanoparticles for Use in Targeting Vascular Receptors,” filed Jul. 31, 2001; and U.S. Provisional Patent Application Serial No. 60/312,435, “Targeted Lipid Constructs For Radiotherapeutic Treatment Of Tumors,” filed Aug. 15, 2001.

### STATEMENT REGARDING FEDERALLY-SPONSORED RESEARCH AND DEVELOPMENT

Statement under MPEP 310. The U.S. Government has a paid-up license in this invention and the right in limited circumstances to require the patent owner to license others on reasonable terms as provided for by the terms of NIH/NCI P20 center grant (CA86312).

Part of the work performed during development of this invention utilized U.S. Government funds. The U.S. Government has certain rights in this invention. This research was supported in part by the NIH/NCI P20 center grant (CA86312).

### FIELD OF THE INVENTION

The present invention concerns targeted agents suitable for a number of in vitro and in vivo applications, including therapeutics, imaging and diagnostics. More particularly, the present invention is concerned with macromolcules having more than one targeting and/or therapeutic entity.

### BACKGROUND OF THE INVENTION

Cancer remains one of the leading causes of death in the industrialized world. In the United States, cancer is the second most common cause of death after heart disease, accounting for approximately one-quarter of the deaths in 1997. Clearly, new and effective treatments for cancer will provide significant health benefits. Among the wide variety of treatments proposed for cancer, targeted therapeutic agents hold considerable promise. In principle, a patient could tolerate much higher doses of a cytotoxic agent if the cytotoxic agent is targeted specifically to cancerous tissue, as healthy tissue should be unaffected or affected to a much smaller extent than the pathological tissue.

Therapeutic Agents

A vast number of therapeutic agents are available for the treatment of cancer. Only a few of interest are discussed here. The anthracycline antibiotic doxorubicin (doxorubicin) and its derivatives, as well as other cationic anthracyclines currently are of great clinical interest in the treatment of cancer, including leukemias and solid tumors. Doxorubicin has wide activity against a number of human neoplasms and is used extensively both as a single agent and in combination regimens. Doxorubicin can be administered in its free form, however, this use of free doxorubicin is linked to toxicity in the form of both an acute and a chronic form of cardiomyopathy. There are two US Food and Drug Administration approved liposomal formulations of doxorubicin currently available, with several additional liposomal formulations being researched either in the laboratory or in clinical trials. These liposomal formulations reduce the toxicity of doxorubicin, as these systems tend to sequester the drug away from organs such as the heart, with greater accumulation in liver, spleen and tumors. Overall, the use of liposomal doxorubicin allows for a greater lifetime cumulative dose of doxorubicin to be administered.

The taxanes are a group of drugs that includes paclitaxel (Taxol®) and docetaxel (Taxotere®), which are used in the treatment of cancer. Taxanes block cell division by the promotion and stabilization of microtubule assemblies. This induced stability dispruts the kinetics and equilibrium of microtubule-dependent cytoplasmic structures that are required for such functions as mitosis, maintenance of cellular morphology, shape changes, neurite formation, locomotion, and secretion, thereby damaging the cells. In December 1992, the U.S. Food and Drug Administration (FDA) approved the use of paclitaxel for ovarian cancer that was resistant to treatment (refractory). Paclitaxel was later approved as initial treatment for ovarian cancer in combination with cisplatin. Women with epithelial ovarian cancer are now generally treated with surgery followed by a taxane and a platinum (another type of anticancer drug). The FDA has also approved paclitaxel for the treatment of breast cancer that recurred within 6 months after adjuvant chemotherapy (chemotherapy that is given after the primary treatment to enhance the effectiveness of the primary treatment), or that spread (metastasized) to nearby lymph nodes or other parts of the body. Paclitaxel is also used for other cancers, including AIDS-related Kaposi's sarcoma and lung cancer.

Docetaxel, a compound that is structurally similar to paclitaxel, has been approved by the FDA to treat advanced breast, lung, and ovarian cancer. Both paclitaxel and docetaxel have unpleasant side effects, and neither is currently available in a liposomal formulation.

Camptothecin and topotecan are other therapeutic agents which exhibit an in vivo antitumor effect, thought to be mediated through the inhibition of angiogenesis. Clements, et al., Cancer Chemother. Pharmacol. (1999) 44:411-16. This publication, and all other patents, patent applications, and publications referred to herein are incorporated by reference herein in their entirety.

Integrins

The integrins are a class of proteins involved in the attachment of cells to matrix via RGD peptide sequences. Ruoslahti & Pierschbacher, Science (1987) 238:491-497. Their expression has been closely associated with many major disease processes involved in the formation of new blood vessels (angiogenesis) such as, osteoporosis, rheumatoid arthritis, macular degeneration and cancer. Folkman, Nature Medicine (1995) 1(1):27-31. The inhibition of the integrins is a new strategy to treat these diseases by either interfering directly with the function of these proteins (anti-angiogenesis) and/or the use of the integrins as an anchor for the delivery of pharmaceutical agents (vascular targeting). Schmitzer, New Eng. J. Med. (1998) 339(7):472-474; Eliceiri, & Cheresh, J. Clin. Invest. (1999) 103(9):1227-1230. Multivalency is a potentially powerful strategy for increasing the avidity of molecules for cell surface receptors. Mammen, et al., Angew. Chem. Int. Ed. (1998) 37:2754-2794. Polymers have been synthesized that contain multivalent arrays of RGD peptides and these materials have shown increased avidity to the integrin is in in vitro assays. Saiki, et al., Cancer Res. (1989) 49(14):3815-3822; Komazawa, et al., J Bioact. Compat. Polym. (1993) 8:258-274; Oku, et al., Life Sci. (1996) 58(24):2263-2270; Kurohane, et al., Life Sci. (2000) 63(3):273-81; Maynard, et al., J. Am. Chem. Soc. (2001) 123:1275-1279. These materials also have been used to inhibit lung and liver metastasis in vivo in animal tumor models. To date no multivalent materials bearing ligands that mimic RGD have been designed for inhibiting the integrins.

Currently at least eleven different α subunits have been identified and at least six different β subunits have been identified. The various a subunits can combine with various β subunits to form distinct integrins. The integrin identified as αᵥβ₃ (also known as the vitronectin receptor) has been identified as an integrin that plays a role in various conditions or disease states including but not limited to tumor metastasis, solid tumor growth (neoplasia), osteoporosis, Paget's disease, humoral hypercalcemia of malignancy, angiogenesis, including tumor angiogenesis, antiangiogenesis, retinopathy, macular degeneration, arthritis, including rheumatoid arthritis, periodontal disease, psoriasis and smooth muscle cell migration (e.g., restenosis). Additionally, it has been found that such integrin inhibiting agents would be useful as antivirals, antifungals and antimicrobials. Thus, therapeutic agents that selectively inhibit or antagonize αᵥβ₃ would be beneficial for treating such conditions. It has been shown that the αᵥβ₃ integrin binds to a number of Arg-Gly-Asp (RGD) containing matrix molecules, such as fibrinogen (Bennett et al., Proc. Natl. Acad. Sci. USA, Vol. 80 (1983) 2417), fibronectin (Ginsberg et al., J. Clin. Invest., Vol. 71 (1983) 619-624), and von Willebrand factor (Ruggeri et al., Proc. Natl. Acad. Sci. USA, Vol. 79 (1982) 6038). Compounds containing the RGD sequence mimic extracellular matrix ligands so as to bind to cell surface receptors. However, it is also known that RGD peptides in general are non-selective for RGD dependent integrins. For example, most RGD peptides that bind to αᵥβ₃ also bind to αᵥβ₅, αᵥβ₁, and α_{IIb}β_{IIIa}. Antagonism of platelet α_{IIb}β_{IIIa} (also known as the fibrinogen receptor) is known to block platelet aggregation in humans.

A number of anti-integrin antibodies are known. Doerr, et al., J. Biol. Chem. 1996 271:2443 reported that a blocking antibody to αᵥβ₅ integrin in vitro inhibits the migration of MCF-7 human breast cancer cells in response to stimulation from IGF-1. Gui et al., British J. Surgery 1995 82:1192, report that antibodies against αᵥβ₁ and αᵥβ₅ inhibit in vitro chemoinvasion by human breast cancer carcinoma cell lines Hs578T and MDA-MB-231. Lehman et al., Cancer Research 1994 54:2102 show that a monoclonal antibody (69-6-5) reacts with several αᵥ integrins including αᵥβ₃ and inhibits colon carcinoma cell adhesion to a number of substrates, including vitronectin. Brooks et al., Science 1994 264:569 show that blockade of integrin activity with an anti-αᵥβ₃ monoclonal antibody inhibits tumor-induced angiogenesis of chick chorioallantoic membranes by human M21 melanoma fragments. Chuntharapai, et al., Exp. Cell. Res. 1993 205:345 disclose monoclonal antibodies 9G2.1.3 and IOC4.1.3 which recognize the αᵥβ₃ complex, the latter monoclonal antibody is said to bind weakly or not at all to tissues expressing αᵥβ₃ with the exception of osteoclasts and was suggested to be useful for in vivo therapy of bone disease. The former monoclonal antibody is suggested to have potential as a therapeutic agent in some cancers.

Ginsberg et al., U.S. Pat. No. 5,306,620 disclose antibodies that react with integrin so that the binding affinity of integrin for ligands is increased. As such these monoclonal antibodies are said to be useful for preventing metastasis by immobilizing melanoma tumors. Brown, U.S. Pat. No. 5,057,604 discloses the use of monoclonal antibodies to αᵥβ₃ integrins that inhibit RGD-mediated phagocytosis enhancement by binding to a receptor that recognizes RGD sequence containing proteins. Plow et al., U.S. Pat. No. 5,149,780 disclose a protein homologous to the RGD epitope of integrin β subunits and a monoclonal antibody that inhibits integrin-ligand binding by binding to the β₃ subunit. That action is said to be of use in therapies for adhesion-initiated human responses such as coagulation and some inflammatory responses.

Carron, U.S. Pat. No. 6,171,588, describe monoclonal antibodies which can be used in a method for blocking αᵥβ₃-mediated events such as cell adhesion, osteoclast-mediated bone resorption, restenosis, ocular neovascularization and growth of hemangiomas, as well as neoplastic cell or tumor growth and dissemination. Other uses described are antibody-mediated targeting and delivery of therapeutics for disrupting or killing αᵥβ₃ bearing neoplasms and tumor-related vascular beds. In addition, the inventive monoclonal antibodies can be used for visualization or imaging of αᵥβ₃ -bearing neoplasms or tumor-related vascular beds by NMR or immunoscintigraphy.

VEGF

A number of angiogenic growth factors have been described to date among which vascular endothelial growth factor (VEGF) appears to play a key role as a positive regulator of physiological and pathological angiogenesis (reviewed in Brown et al. (1997) in Control of Angiogenesis (Goldberg and Rosen, eds.), Birkhauser, Basel, 233-269; Thomas (1996) J. Biol. Chem. 271:603-606; Neufeld et al. (1999) FASEB J. 13:9-22). VEGF is a secreted disulfide-linked homodimer that selectively stimulates endothelial cells to proliferate, migrate, and produce matrix-degrading enzymes (Conn et al. (1990) Proc. Natl. Acad. Sci. U.S.A. 87:1323-1327; Ferrara and Henzel (1989) Biochem. Biophys. Res. Commun. 161:851-858; Gospodarowicz et al. (1989) Proc. Natl. Acad. Sci. U.S.A. 86:7311-7315; Pepper et al. (1991) Biochem. Biophys. Res. Commun. 181:902-906; Unemori et al. (1992) J. Cell. Physiol. 153:557-562), all of which are processes required for the formation of new vessels. In addition to being the only known endothelial cell specific mitogen, VEGF is unique among angiogenic growth factors in its ability to induce a transient increase in blood vessel permeability to macromolecules (hence its original and alternative name, vascular permeability factor) (Dvorak et al.(1979) J. Immunol. 122:166-174; Senger et al.(1983) Science 219:983-985; Senger et al.(l 986) Cancer Res. 46:5629-5632). Increased vascular permeability and the resulting deposition of plasma proteins in the extravascular space assists the new vessel formation by providing a provisional matrix for the migration of endothelial cells (Dvorak et al.(1995) Am. J. Pathol. 146:1029-1039). Hyperpermeability is indeed a characteristic feature of new vessels, including those associated with tumors (Dvorak et al.(1995) Am. J. Pathol. 146:1029-1039). Furthermore, compensatory angiogenesis induced by tissue hypoxia is now known to be mediated by VEGF (Levy et al.(1996) J. Biol. Chem. 271:2746-2753); Shweiki et al. (1992) Nature 359:843-845).

VEGF is produced and secreted in varying amounts by virtually all tumor cells (Brown et al. (1997) in Control of Angiogenesis (Goldberg and Rosen, eds.), Birkhauser, Basel:233-269). Direct evidence that VEGF and its receptors contribute to tumor growth was recently obtained by a demonstration that the growth of human tumor xenografts in nude mice could be inhibited by neutralizing antibodies to VEGF (Kim et al. (1993) Nature 362:841-844), by the expression of dominant-negative VEGFR2 (Millauer et al. (1996) Cancer Res. 56:1615-1620; Millauer et al. (1994) Nature 367:576-579), by low molecular weight inhibitors of VEGF receptor inhibitors (Strawn et al. (1966) Cancer Res. 56:3540-3545), or by the expression of antisense sequence to VEGF mRNA (Saleh et al. (1996) Cancer Res. 56:393-401). Importantly, the incidence of tumor metastases was also found to be dramatically reduced by VEGF antagonists (Asano et al. (1995) Cancer Res. 55:5296-5301; Warren et al. (1995) J. Clin. Invest. 95:1789-1797; Claffey et al. (1996) Cancer Res. 56:172-181; Melnyk et al. (1996) Cancer Res. 56:921-924). Inhibitors of VEGF signaling may thus have broad clinical utility as anticancer agents. In addition to cancer, as noted above, other proliferative diseases characterized by excessive neovascularization such as psoriasis, age-related macular degeneration, diabetic retinopathy and rheumatoid arthritis could be treated with antagonists of VEGF signaling.

VEGF occurs in several forms (VEGF-121, VEGF-145, VEGF-165, VEGF-189, VEGF-206) as a result of alternative splicing of the VEGF gene that consists of eight exons (Houck et al. (1991) Mol. Endocrin. 5:1806-1814; Tischer et al. (1991) J. Biol. Chem. 266:11947-11954; Poltorak et al. (1997) J. Biol. Chem. 272:7151-7158). The three smaller forms are diffusable, while the larger two forms remain predominantly localized to the cell membrane as a consequence of their high affinity for heparin. VEGF-165 and VEGF-145 also bind to heparin (as a consequence of containing basic exon 7- and exon 6-encoded domains, respectively), albeit with somewhat lower affinity compared with VEGF-189 (that contains both exons 6 and 7). VEGF-165 appears to be the most abundant form in most tissues (Houck et al. (1991) Mol. Endocrinol. 5:1806-1814; Carmeliet et al. (1999) Nature Med. 5:495-502). VEGF-121, the only alternatively spliced form that does not bind to heparin, appears to have a somewhat lower affinity for the receptors (Gitay-Goren et al. (1996) J. Biol. Chem. 271:5519-5523) as well as lower mitogenic potency (Keyt et al. (1996) J. Biol. Chem. 271:7788-7795).

Biological effects of VEGF are mediated by two homologous tyrosine kinase receptors, Flt-1 (VEGFR1) and Flk-1/KDR (VEGFR2) whose expression is highly restricted to cells of endothelial origin (de Vries et al. (1992) Science 255:989-991; Millauer et al. (1993) Cell 72:835-846; Terman et al. (1991) Oncogene 6:519-524). Both receptors have an extracellular domain consisting of seven IgG-like domains, a transmembrane domain and an intracellular tyrosine kinase domain. The affinity of VEGFR1 for VEGF (K_{d}=1-20 pM) is higher compared to that of VEGFR2 (K_{d}=50-770 pM) (Brown et al. (1997) in Regulation of Angiogenesis, supra; de Vries et al. (1992) Science 255:989-991; Terman et al. (1992) Biochem. Biophys. Res. Commun. 187:1579-1586). In human umbilical cord endothelial cells (HUVECs) in 2-dimensional culture, VEGFR2 is by far the more abundant receptor (Brown et al. (1997) in Regulation of Angiogenesis, supra). In vivo, however, in quiescent endothelial cells, both receptors are expressed at low levels (Kremer et al. (1997) Cancer Res. 57:3852-3859; Barleon et al. (1997) Cancer Res. 57:5421-5425).

Both receptors are substantially upregulated when endothelial cells are activated by a variety of stimuli. Hypoxia, for example, induces an increase in expression of both VEGFR1 and VEGFR2 in endothelial cells (Tuder et al. (1995) J. Clin. Invest. 95:1798-1807; Gerber et al. (1997) J. Biol. Chem. 272:23659-23667; Brogi et al. (1996) J. Clin. Invest. 97:469-476; Kremer et al. (1997) Cancer Res. 57:3852-3859). For VEGFR1, hypoxia leads to both direct activation via the flt-1 promoter that contains the hypoxia-inducible-factor-1 (HIF-1) consensus binding site (Gerber et al. (1997) J. Biol. Chem., supra) and indirect activation via hypoxia-induced VEGF (Barleon et al. (1997) Cancer Res., supra). VEGF-induced upregulation of VEGFR1 is mediated by both VEGFR1 and VEGFR2 (Barleon et al. (1997) Cancer Res., supra). VEGFR2 is upregulated by VEGF (through VEGFR2, but not VEGFR1) (Kremer et al. (1997) Cancer Res., supra; Wilting et al. (1996) Dev. Biol. 176:76-85) and possibly by a yet unidentified factor in hypoxia-conditioned media from myoblasts (Brogi et al. (1996) J. Clin. Invest., supra). The expression of VEGFR2 in endothelial cells is also upregulated by bFGF and this accounts in part for the synergistic activation of endothelial cells by VEGF and bFGF (Pepper et al. (1998) Exp. Cell Res. 241:414-425). In addition, since both kdr and flt-1 promoters contain a cis-acting fluid shear-stress-responsive element, VEGFR1 and VEGFR2 expression may be sensitive to variations in blood flow (Tuder et al. (1995) J. Clin. Invest., supra).

Experiments using porcine aortic endothelial (PAE) cells transfected with the flt-1 or kdr receptor genes have suggested that VEGFR2 is the primary transducer in endothelial cells of VEGF-mediated signals related to changes in cell morphology and mitogenicity (Waltenberger et al. (1994) J. Biol. Chem. 269:26988-26995). In the same study, stimulation of flt-1-transfected PAE cells with VEGF did not appear to produce detectable changes. More recently, however, it was demonstrated that VEGF signaling through VEGFR1 induces migration of monocytes and upregulation of tissue factor expression in both endothelial cells and monocytes (Clauss et al. (1996) J. Biol. Chem. 271:17629-17634; Barleon et al. (1996) Blood 87:3336-3343). Based on the observation that the extracellular domain of VEGFR2 is retained on a cation exchange resin only in the presence of VEGFR1 and that the VEGFR2 retention is enhanced when both VEGFR1 and VEGF were present, Kendall et al. have concluded that the two receptors have some affinity for one another and that this interaction is stabilized by VEGF (Kendall et al. (1996) Biochem Biophys. Res. Commun. 226:324-328). When both receptors are expressed on cell surface, it appears likely that the VEGFR1/R2 heterodimer constitutes at least a fraction of the binding-competent VEGF receptor.

Although VEGFR1 and VEGFR2 are expressed predominantly on endothelial cells, they have also been detected on some non-endothelial cells. VEGFR1 is expressed on trophoblasts (Charnockjones et al. (1994) Biol. Reprod. 51:524-530), monocytes (Barleon et al. (1996) Blood, supra), hematopoietic stem cells and megakaryocytes/platelets (Katoh et al. Cancer Res. 55:5687-5692), renal mesangial cells (Takahashi et al. (1995) Biochem. Biophys. Res. Commun. 209:218-226) and pericytes (Yamagishi et al. (1999) Lab. Invest. 79:501-509). In monocytes, VEGFR1 is responsible for the VEGF-mediated induction of migration and tissue factor expression (Clauss et al. (1996) J. Biol. Chem., supra; Barleon et al. (1996) Blood, supra; Hiratsuka et al. (1998) Proc. Natl. Acad. Sci., supra). In pericytes, VEGFR1 may mediate the recently described ability of VEGF to act as a mitogen and chemotactic factor (Yamagishi et al. (1999) Lab. Invest., supra). The role of VEGFR1 in trophoblasts and mesangial cells remains to be elucidated. The expression of VEGFR2 has been detected on hematopoietic stem cells, megakaryocytes/platelets and retinal progenitor cells (Katoh et al. (1995) Cancer Res. 55:5687-5692; Yang et al. (1996) J. Neurosci. 16:6089-6099). VEGFR1 and VEGFR2 expression has also been reported on malignant cells including leukemia cells (Katoh et al. (1995) Cancer Res., supra) and melanoma cells (Gitay-Goren et al. (1993) Biochem. Biophys. Res. Commun. 190:702-709).

Targeted Therapeutics

Examples of the targeted therapeutic approach have been described in various patent publications and scientific articles. International Patent Application WO 93/17715 describes antibodies carrying diagnostic or therapeutic agents targeted to the vasculature of solid tumor masses through recognition of tumor vasculature-associated antigens. International Patent Application WO 96/01653 and U.S. Pat. No. 5,877,289 describe methods and compositions for in vivo coagulation of tumor vasculature through the site-specific delivery of a coagulant using an antibody, while International Patent Application WO 98/31394 describes use of Tissue Factor compositions for coagulation and tumor treatment. International Patent Application WO 93/18793 and U.S. Pat. Nos. 5,762,918 and 5,474,765 describe steroids linked to polyanionic polymers which bind to vascular endothelial cells. International Patent Application WO 91/07941 and U.S. Pat. No. 5,165,923 describe toxins, such as ricin A, bound to antibodies against tumor cells. U.S. Pat. Nos. 5,660,827, 5,776,427, 5,855,866, and 5,863,538 also disclose methods of treating tumor vasculature. International Patent Application WO 98/10795 and WO 99/13329 describe tumor homing molecules, which can be used to target drugs to tumors.

In Tabata, et al., Int. J. Cancer 1999 82:737-42, antibodies are used to deliver radioactive isotopes to proliferating blood vessels. Ruoslahti & Rajotte, Annu. Rev. Immunol. 2000 18:813-27; Ruoslahti, Adv. Cancer Res. 1999 76:1-20, review strategies for targeting therapeutic agents to angiogenic neovasculature, while Arap, et al., Science 1998 279:377-80 describe selection of peptides which target tumor blood vessels.

It should be noted that the typical arrangement used in such systems is to link the targeting entity to the therapeutic entity via a single bond or a relatively short chemical linker. Examples of such linkers include SMCC (succinimidyl 4-[N-maleimidomethyl]cyclohexane-1-carboxylate) or the linkers disclosed in U.S. Pat. No. 4,880,935, and oligopeptide spacers. Carbodiimides and N-hydroxysuccinimide reagents have been used to directly join therapeutic and targeting entities with the appropriate reactive chemical groups.

The use of cationic organic molecules to deliver heterologous genes in gene therapy procedures has been reported in the literature. Not all cationic compounds will complex with DNA and facilitate gene transfer. Currently, a primary strategy is routine screening of cationic molecules. The types of compounds which have been used in the past include cationic polymers such as polyethyleneamine, ethylene diamine cascade polymers, and polybrene. Proteins, such as polylysine with a net positive charge, have also been used. The largest group of compounds, cationic lipids; includes DOTMA, DOTAP, DMRIE, DC-chol, and DOSPA. All of these agents have proven effective but suffer from potential problems such as toxicity and expense in the production of the agents. Cationic liposomes are currently the most popular system for gene transfection studies. Cationic liposomes serve two functions: protect DNA from degradation and increase the amount of DNA entering the cell. While the mechanisms describing how cationic liposomes function have not been fully delineated, such liposomes have proven useful in both in vitro and in vivo studies. However, these liposomes suffer from several important limitations. Such limitations include low transfection efficiencies, expense in production of the lipids, poor colloidal stability when complexed to DNA, and toxicity.

Although conjugates of targeting entities with therapeutic entities via relatively small linkers have attracted much attention, far less attention has been focused on using large particles as linkers. Typically, the linker functions simply to connect the therapeutic and targeting entities, and consideration of linker properties generally focuses on avoiding interference with the entities linked, for example, avoiding a linkage point in the antigen binding site of an immunoglobulin.

Large particulate assemblies of biologically compatible materials, such as liposomes, have been used as carriers for administration of drugs and paramagnetic contrast agents. U.S. Pat. Nos. 5,077,057 and 5,277,914 teach preparation of liposome or lipidic particle suspensions having particles of a defined size, particularly lipids soluble in an aprotic solvent, for delivery of drugs having poor aqueous solubility. U.S. Pat. No. 4,544,545 teaches phospholipid liposomes having an outer layer including a modified, cholesterol derivative to render the liposome more specific for a preselected organ. U.S. Pat. No. 5,213,804 teaches liposome compositions containing an entrapped agent, such as a drug, which are composed of vesicle-forming lipids and 1 to 20 mole percent of a vesicle-forming lipid derivatized with hydrophilic biocompatible polymer and sized to control its biodistribution and recirculatory half life. U.S. Pat. No. 5,246,707 teaches phospholipid-coated microcrystalline particles of bioactive material to control the rate of release of entrapped water-soluble biomolecules, such as proteins and polypeptides. U.S. Pat. No. 5,158,760 teaches liposome encapsulated radioactive labeled proteins, such as hemoglobin.

U.S. Pat. Nos. 5,512,294 and 6,090,408, and 6,132,764 (the contents of which are hereby incorporated by reference herein) describe the use of polymerized liposomes for various biological applications. One listed embodiment is to targeted polymerized liposomes which may be linked to or may encapsulate a therapeutic compound, (e.g. proteins, hormones or drugs), for directed delivery of a treatment agent to specific biological locations for localized treatment. Other publications describing liposomal compositions include U.S. Pat. Nos. 5,663,387, 5,494,803, and 5,466,467. Liposomes containing polymerized lipids for non-covalent immobilization of proteins and enzymes are described in Storrs et al., “Paramagnetic Polymerized Liposomes: Synthesis, Characterization, and Applications for Magnetic Resonance Imaging,” J. Am. Chem. Soc. (1995) 117(28):7301-7306; and Storrs et al., “Paramagnetic Polymerized Liposomes as New Recirculating MR Contrast Agents,” JMRI (1995) 5(6):719-724. Wu et al., “Metal-Chelate-Dendrimer-Antibody Constructs for Use in Radioimmunotherapy and Imaging,” Bioorganic and Medicinal Chemistry Letters (1994) 4(3):449-454, is a publication directed to dendrimer-based compounds.

Stabilization

The association of liposomes with polymeric compounds in order to avoid rapid clearance in the liver, or for other stabilizing effects, has been described. For example, Dadey, U.S. Pat. No. 5,935,599 described polymer-associated liposomes containing a liposome, and a polymer having a plurality of anionic moieties in a salt form. The polymer may be synthetic or naturally-occurring. The polymer-associated liposomes remain in the vascular system for an extended period of time.

Polysaccharides are one class of polymeric stabilizer. Calvo Salve, et al., U.S. Pat. No. 5,843,509 describe the stabilization of colloidal systems through the formation of lipid-polysaccharide complexes and development of a procedure for the preparation of colloidal systems involving a combination of two ingredients: a water soluble and positively charged polysaccharide and a negatively-charged phospholipid. Stabilization occurs through the formation, at the interface, of an ionic complex: aminopolysaccharide-phospholipid. The polysaccharides utilized by Calvo Salve, et al., include chitin and chitosan.

Dextran is another polysaccharide whose stabilizing properties have been investigated. Cansell, et al., J. Biomed. Mater. Res. 1999, 44:140-48, report that dextran or functionalized dextran was hydrophobized with cholesterol, which anchors in the lipid bilayer of liposomes during liposome formation, resulting in a liposome coated with dextran. These liposomes interacted specifically with human endothelial cells in culture. In Letourneur, et al., J. Controlled Release 2000, 65:83-91, the antiproliferative functionalized dextran-coated liposomes were used as a targeting agent for vascular smooth muscle cells. Ullman, et al. Proc. Nat. Acad. Sci 91:5426-30 (1994) and Ullman, et al., Clin. Chem. 42:1518-26 (1996) describe the coating of polystyrene beads with dextran and the attachment of ligands, nucleic acids, and proteins to the dextran-polystyrene complexes.

Dextran has also been used to coat metal nanoparticles, and such nanoparticles have been used primarily as imaging agents. For example, Moore, et al., Radiology 2000, 214:568-74, report that in a rodent model, long-circulating dextran-coated iron oxide nanoparticles were taken up preferentially by tumor cells, but also were taken up by tumor-associated macrophages and, to a much lesser extent, endothelial cells in the area of angiogenesis. Groman, et al., U.S. Pat. No. 4,770,183, describe 10-5000 Å superparamagnetic metal oxide particles for use as imaging agents. The particles may be coated with dextran or other suitable polymer to optimize both the uptake of the particles and the residence time in the target organ. A dextran-coated iron oxide particle injected into a patient's bloodstream, for example, localizes in the liver. Groman, et al., also report that dextran-coated particles can be preferentially absorbed by healthy cells, with less uptake into cancerous cells.

Imaging

Magnetic resonance imaging (MRI) is an imaging technique which, unlike X-rays, does not involve ionizing radiation. MRI may be used for producing cross-sectional images of the body in a variety of scanning planes such as, for example, axial, coronal, sagittal or orthogonal. MRI employs a magnetic field, radio-frequency energy and magnetic field gradients to make images of the body. The contrast or signal intensity differences between tissues mainly reflect the T1 (longitudinal) and T2 (transverse) relaxation values and the proton density in the tissues. To change the signal intensity in a region of a patient by the use of a contrast medium, several possible approaches are available. For example, a contrast medium may be designed to change either the T1, the T2 or the proton density.

Generally speaking, MRI requires the use of contrast agents. If MRI is performed without employing a contrast agent, differentiation of the tissue of interest from the surrounding tissues in the resulting image may be difficult. In the past, attention has focused primarily on paramagnetic contrast agents for MRI. Paramagnetic contrast agents involve materials which contain unpaired electrons. The unpaired electrons act as small magnets within the main magnetic field to increase the rate of longitudinal (T1) and transverse (T2) relaxation. Paramagnetic contrast agents typically comprise metal ions, for example, transition metal ions, which provide a source of unpaired electrons. However, these metal ions are also generally highly toxic. For example, ferrites often cause symptoms of nausea after oral administration, as well as flatulence and a transient rise in serum iron. The gadolinium ion, which is complexed in Gd-DTPA, is highly toxic in free form. The various environments of the gastrointestinal tract, including increased acidity (lower pH) in the stomach and increased alkalinity (higher pH) in the intestines, may increase the likelihood of decoupling and separation of the free ion from the complex. In an effort to decrease toxicity, the metal ions are typically chelated with ligands.

Ultrasound is another valuable diagnostic imaging technique for studying various areas of the body, including, for example, the vasculature, such as tissue microvasculature. Ultrasound provides certain advantages over other diagnostic techniques. For example, diagnostic techniques involving nuclear medicine and X-rays generally involve exposure of the patient to ionizing electron radiation. Such radiation can cause damage to subcellular material, including deoxyribonucleic acid (DNA), ribonucleic acid (RNA) and proteins. Ultrasound does not involve such potentially damaging radiation. In addition, ultrasound is inexpensive relative to other diagnostic techniques, including CT and MRI, which require elaborate and expensive equipment.

Ultrasound involves the exposure of a patient to sound waves. Generally, the sound waves dissipate due to absorption by body tissue, penetrate through the tissue or reflect off of the tissue. The reflection of sound waves off of tissue, generally referred to as backscatter or reflectivity, forms the basis for developing an ultrasound image. In this connection, sound waves reflect differentially from different body tissues. This differential reflection is due to various factors, including the constituents and the density of the particular tissue being observed. Ultrasound involves the detection of the differentially reflected waves, generally with a transducer that can detect sound waves having a frequency of one to ten megahertz (MHz). The detected waves can be integrated into an image which is quantitated and the quantitated waves converted into an image of the tissue being studied.

As with the diagnostic techniques discussed above, ultrasound also generally involves the use of contrast agents. Exemplary contrast agents include, for example, suspensions of solid particles, emulsified liquid droplets, and gas-filled bubbles (see, e.g., Hilmann et al., U.S. Pat. No. 4,466,442, and published International Patent Applications WO 92/17212 and WO 92/21382). Widder et al., published application EP-A-0 324 938, disclose stabilized microbubble-type ultrasonic imaging agents produced from heat-denaturable biocompatible protein, for example, albumin, hemoglobin, and collagen.

The reflection of sound from a liquid-gas interface is extremely efficient. Accordingly, liposomes or vesicles, including gas-filled bubbles, are useful as contrast agents. As discussed more fully hereinafter, the effectiveness of liposomes as contrast agents depends upon various factors, including, for example, the size and/or elasticity of the bubble.

Many of the liposomes disclosed in the prior art have undesirably poor stability. Thus, the prior art liposomes are more likely to rupture in vivo resulting, for example, in the untimely release of any therapeutic and/or diagnostic agent contained therein. Various studies have been conducted in an attempt to improve liposome stability. Such studies have included, for example, the preparation of liposomes in which the membranes or walls thereof comprise proteins, such as albumin, or materials which are apparently strengthened via crosslinking. See, e.g., Klaveness et al., WO 92/17212, in which there are disclosed liposomes which comprise proteins crosslinked with biodegradable crosslinking agents. A presentation was made by Moseley et al., at a 1991 Napa, Calif. meeting of the Society for Magnetic Resonance in Medicine, which is summarized in an abstract entitled “Microbubbles: A Novel MR Susceptibility Contrast Agent.” The microbubbles described by Moseley et al. comprise air coated with a shell of human albumin. Alternatively, membranes can comprise compounds which are not proteins but which are crosslinked with biocompatible compounds. See, e.g., Klaveness et al., WO 92/17436, WO 93/17718 and WO 92/21382.

Prior art techniques for stabilizing liposomes, including the use of proteins in the outer membrane, suffer from various drawbacks. The use in membranes of proteins, such as albumin, can impart rigidity to the walls of the bubbles. This results in bubbles having educed elasticity and, therefore, a decreased ability to deform and pass through capillaries. Thus, there is a greater likelihood of occlusion of vessels with prior art contrast agents that involve proteins.

### SUMMARY OF THE INVENTION

The present invention provides a targeted therapeutic agent comprising a linking carrier, a therapeutic entity associated with the linking carrier, and at least one targeting entity. In some embodiments, the agent has three or more targeting entities, ten or more targeting entities, 100 or more targeting entities, and 1000 or more targeting entities, or is present at a concentration from 0.1 to 10 mole percent of the targeting agent. The linking carrier may be a macromolecule, including a liposome, a polymerized vesicle, a dendrimer, and a block copolymer, among others. In some embodiments, the linking carrier comprises a phosphatidylcholine derivative.

The targeting entity may be associated with the lipid construct by covalent or non-covalent means. The targeting entity may target the lipid construct to a cell surface, or may have a vascular target, a tumor cell target.

In some embodiments, the targeting entity is an integrin-specific molecule, such as an RGD peptide, or RGD petidomimetic, including 3-{4-[2-(3,4,5,6-tetrahydropyrimidin-2-ylamino)-ethyloxy]-benzoylamino}-2(S)-benzene-sulfonyl-aminopropionic acid.

In particular, the present invention provides a targeted therapeutic agent comprising more than one 3-{4-[2-(3,4,5,6-tetrahydropyrimidin-2-ylamino)-ethyloxy]-benzoylamino}-2(S)-benzene-sulfonyl-aminopropionic acid moiety. The targeted therapeutic agent may include a compound of the formula:

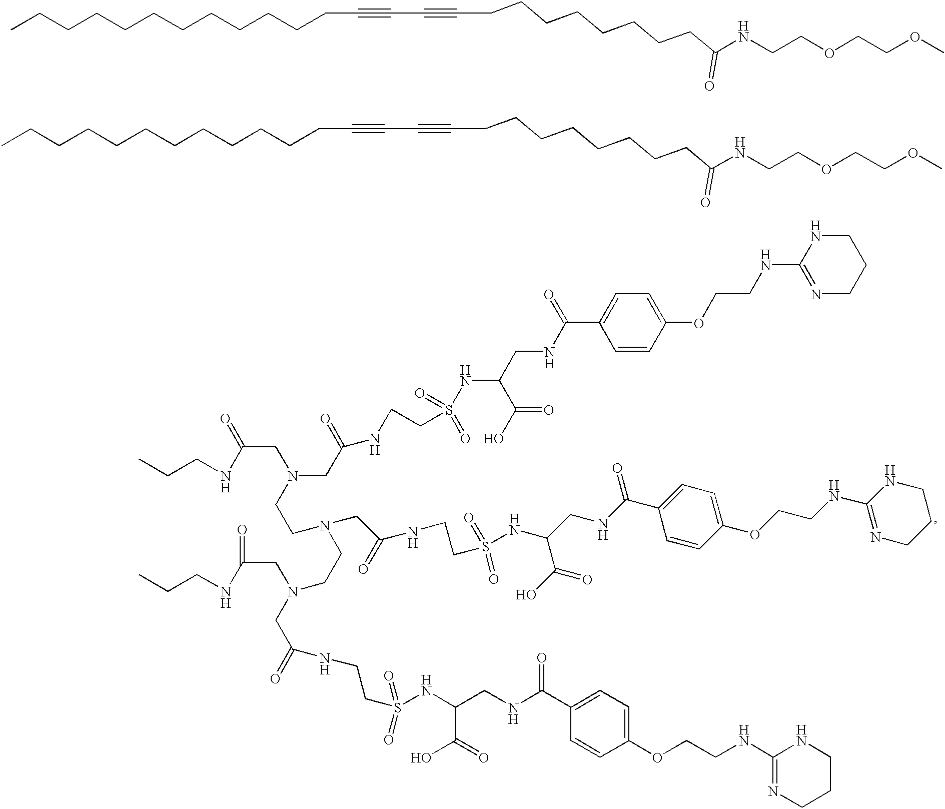

wherein the compound is associated with the lipid construct by non-covalent or covalent means. This compound is also provided by the present invention.

In some embodiments, the targeting entity is a tyrosine kinase specific molecule, such as the compounds AG1433 or SU1498.

In other embodiments, the targeting entity has a target selected from the group consisting of P-selectin, E-selectin, pleiotropin, G-protein coupled receptors, endosialin, endoglin, VEGF receptors, PDGF receptor, EGF receptor, FGF receptors, the matrix metalloproteases including MMP2 and MMP9, and prostate specific membrane antigen (PSMA).

In other embodiments, the targeting entity is an enzyme modulator.

In further embodiments, the targeted therapeutic agent further comprises a stabilizing entity, such as natural polymer, a semi-synthetic polymer, and a synthetic polymer, such as dextran, modified dextran, and poly (ethylene imine). In some embodiments, the stabilizing entity provides the capacity for multivalency.

The targeted therapeutic agent may comprise a therapeutic entity which is present at a concentration of about 1% to about 20%. The therapeutic entity may be doxorubicin, daunorubicin, epirubin, or idarubicin., or a taxane compound, such as paclitaxel or docetaxel, or other agents, such as camptothecin or topotecan.

In some embodiments, the invention provides a targeted therapeutic agent, comprising doxorubicin and 3-{4-[2-(3,4,5,6-tetrahydropyrimidin-2-ylamino)-ethyloxy]-benzoylamino}-2(S)-benzene-sulfonyl-aminopropionic acid.

In still other embodiments, the invention provides a method of preparing a targeted therapeutic agent, comprising providing a targeted lipid construct, said targeted lipid construct comprising more than one targeting entity, and associating a therapeutic entity within the lipid construct. The lipid construct selected from the group consisting of liposomes, micelles, vesicles, and polymerized liposomes. The targeting entity may be doxorubicin, daunorubicin, epirubin, idarubicin, a taxane compound, such as paclitaxel or docetaxel, or other therapeutic entity, such as camptothecin or topotecan.

In a further embodiment, the invention provides a method of treating a patient in need thereof comprising administering an effective amount of a pharmaceutical composition comprising a linking carrier, said linking carrier comprising at least one targeting entity, and an associated therapeutic entity to a patient need thereof.

### BRIEF DESCRIPTION OF THE FIGURES

FIGS. 1A-I shows schematics of exemplary therapeutic constructs of the present invention. Lipid constructs that form micelles or vesicles are preferred carriers.

FIG. 1A shows a polymer-coated carrier with targeting agent 54 and an encapsulated therapeutic agent 56. The polymer coat 52 is external relative to vesicle 50.

FIG. 1B shows a polymer-coated carrier with targeting agent 54 and a therapeutic agent 56 that is associated with the components of the vesicle. The polymer coat 52 is external relative to vesicle 50.

FIG. 1C shows carrier 50 with targeting agent 54 and an encapsulated therapeutic agent 56.

FIG. 1D shows carrier 50 with targeting agent 54 and a therapeutic agent 56 that is associated with the components of the vesicle.

FIG. 1E shows a polymer-coated carrier with targeting agent 54 and a therapeutic agent 56 that is associated with the surface of the vesicle. The polymer coat 52 is external relative to vesicle 50.

FIG. 1F shows carrier 50 with targeting agent 54 and therapeutic agent 56, which is attached to the surface of the vesicle by covalent or non-covalent means.

FIG. 1G shows a polymer-coated carrier with a therapeutic agent 56 that is associated with the surface of the vesicle by covalent or non-covalent means. The polymer coat 52 is external relative to vesicle 50.

FIG. 1H shows therapeutic agent 56 attached to the surface of carier 50 by covalent or non-covalent means.

FIG. 1I shows a polymer-coated carrier with targeting agent 54 and therapeutic agent 56 that are associated with the polymer coat by covalent or non-covalent means. The polymer coat 52 is external relative to vesicle 50.

FIG. 2. Coupling of a ligand Z-Y-L where Z is a chemically reactive moiety covalently attached to a spacer Y that is covalently attached to ligand L. This conjugation may require an activating agent such as a carbodiimide derivative or reducing agent.

FIG. 3 shows the structure of N-succinyl-DPPE, sodium salt.

FIG. 4 shows the structure of N-caproylamine-DPPE hydrochloride.

FIGS. 5-15 show exemplary lipids with a variety of functionalites for linking a lipid to a targeting entity or therapeutic entity, and showing various spacer groups.

FIG. 16 shows the synthesis of the integrin antagonist that contains a linker to attach to a lipid for incorporation into polymerized vesicles for multivalent display. The compound was designed to incorporate an ethylamine linker and retain the aminosulfonate that is necessary for binding to the integrins. Conditions: (a) 4N NaOH, 10% NaHCO₃; (b) PCl₅; (c) NMM, THF; (d) LiOH, THF, H₂O; (e) TFA, CH₂Cl₂; (f) HS, EDC, HOBT, NMM; (g) H₂, 10% Pd/C, HOAc, HCl; (h) BOP, Et₃N, DMF, CH₂Cl₂.

FIG. 17 shows the key monomeric lipids 12-16 for use in assembling the polymerized PVs PV1-PV6. The lipids were combined in the ratios as shown in the accompanying table. These compounds were then sonicated, cooled and polymerized by irradiation with UV light (254 nm) for 2 hours and then sterile filtered (0.2 μM). FIGS. 17-30 show lipids for the attachment of targeting agents. These lipids may be used to prepare vesicles for the attachment of targeting or therapeutic agents or both. Some of these lipids may be incorporated into vesicles and then further derivatized in aqueous solution with chemically reactive entities to which targeting agents may be attached. For examples 23-30, R is defined as any lipid, fatty acid, or di- or tri-block copolymer.

FIGS. 18A-18B shows the binding of vesicles containing chelator lipid 15 to αᵥβ₃ integrin-coated 96-well plates. For this assay, vesicles were labled with europium, and time-resolved fluorescence was measured as described in EXAMPLE 5.

FIG. 19 shows the concentration of RGD mimetic 10 required to inhibit 50% of RGD mimetic polymerized vesicles contructs from Table Z. These results were obtained using the integrin-binding assay described in Example 5.

FIGS. 20A-20E showe the use of PVs in imaging tumors in vivo. FIG. 20A shows a schematic of the imaged animal and tumor. FIGS. 20B and 20C are images at 3 hours and 24 hours respectively, after injection of PV1 (targeted PV). FIGS. 20D and 20E are images at 3 hours and 24 hours respectively, after injection of PV4 (control PV).

FIG. 21 shows results from the treatment of endothelial cells and tumor cells in vitro with peptidomimetic-vesicle conjugates containingl % by weight doxorubicin (PM-V-1%Dox) vesicles containing 1% by weight doxorubicin (V-1% Dox), and free doxorubicin at concentrations identical to that used in the vesicles (1% Dox). The cells were treated as described in EXAMPLE 36.

FIG. 22 shows the inhibition of the binding of HRP-labeled fibronectin to the αᵥβ₃ integrin by RGD peptidomimetic (PM) vesicles containing N-succinyl-DPPE (SDPPE), DMPC, DPPC, cholesterol (CH), BisT-PC, RGD peptidomimetic lipid (PML) 1, and paclitaxel (PTX). In this inhibition assay, described in EXAMPLE 38, signal decreases with increasing concentration of vesicles with surface bound RGD peptidomimetic.

FIG. 23 shows the efficacy of integrin-targeted vesicles labeled with yttrium 90 (IA-NP-Y90) in the mouse melanoma model as described in Example 29. Treatment groups include IA (the RGD peptidomimetic 10), IA-NP (RGD-peptidomimetic-polymerized vesicle conjugates), NP-Y90 (polymerized vesicles labeled with yttrium-90), and IA-NP-Y90 (RGD-peptidomimetic-polymerized vesicle conjugates labeled with yttrium-90).

FIG. 24 shows the normalized tumor volume 7 days post treatment sorted by treatment group for the study described in Example 29.

FIG. 25 shows the tumor growth delay data in mouse melanoma study described in example 29 as measured by tumor volume quadrupling time (TVQT).

FIG. 26. Treatment of solid tumors in a mouse melanoma model with integrin targeted dextran-coated polymerized vesicle conjugates labeled with yttrium-90 as described in Example 30.

FIG. 27 shows efficacy in the mouse colon cancer model as described in Example 31. Error bars indicate ±one standard error. Treatment groups include buffer, PM (RGD peptidomimetic 10), PM-PV (RGD peptidomimetic-vesicle conjugates), PV-Y90 (polymerized vesicles labeled with yttrium-90), and PM-PV-Y90 (RGD peptidomimetic-vesicle conjugates labeled with yttrium-90).

FIG. 28: Plot of normalized tumor volume on day 8 sorted by group for the study in Example 31.

FIG. 29 shows the inhibition of the papain-catalyzed hydrolysis of substrate Ala-Phe-Lys-7-aminomethylcoumarin (Biochim. Biophys Acta 1190, 430, (1994)) by N-Acetyl-Leu-Val-Lys-aldehyde (LVK-CHO, J. Med. Chem 36, 1084, (1993)) and N-Acetyl-Leu-Val-Lys-aldehyde-vesicle conjugates (Vesicle-LVK-CHO) described in Example 44.

FIG. 30 shows the inhibition of the papain-catalyzed hydrolysis of substrate Z-Phe-Arg-7-aminomethylcoumarin (Biochem J. 187, 909, (1980)) by N-Acetyl-Leu-Val-Lys-aldehyde (LVK-CHO) and N-Acetyl-Leu-Val-Lys-aldehyde-vesicle conjugates (Vesicle-LVK-CHO) described in Example 44.

FIG. 31 shows the inhibition of the papain-catalyzed hydrolysis of substrate Ala-Phe-Lys-7-aminomethylcoumarin by vesicles by Gly-Phe-Gly-semicarbazone (GFGsc, J. Parasitol. 83, 112, (1997)) and Gly-Phe-Gly-semicarbazone-vesicle conjugates (Vesicle-dex-GFGsc and Vesicle-GFGsc) described in Example 44.

FIG. 32 shows the inhibition of the papain-catalyzed hydrolysis of substrate Z-Phe-Arg-7-aminomethylcoumarin by vesicles by Gly-Phe-Gly-semicarbazone (GFGsc) and Gly-Phe-Gly-semicarbazone-vesicle conjugates (Vesicle-dex-GFGsc and Vesicle-GFGsc) described in Example 44.

FIG. 33 shows the inhibition of the cathepsin-catalyzed hydrolysis of substrate Z-Arg-Arg-7-aminomethylcoumarin (Z-RRamc, Meth. Enzymol. 80, 535 (1981)) by inhibitor Leu-Val-Lys-aldehyde (LVK-CHO) and a vesicle conjugate (Vesicle-LVK-CHO).

FIG. 34 shows compound 18: Arginine-lipid

FIGS. 35A and 35B shows the structures of AG1433 and SU1498, respectively.

FIG. 36. Synthetic scheme for the preparation of compounds in Examples 39-43.

FIG. 37. Synthetic scheme for the preparation of compounds in Examples 46-49.

FIG. 38. Shows the normalized tumor volumes after the treatment of subcutaneous tumors in a syngeneic murine tumor model with sucrose (♦), Ldox (□, liposomal doxorubicin, 10 μg/g doxorubicin), ITL (◯, integrin-targeted liposomes), ITLdox1 (●, integrin-targeted liposomes containing doxorubicin, 1 μg/g doxorubicin), and ITLdox10 (▪, integrin-targeted liposomes containing doxorubicin, 10 μg/g doxorubicin) as described in EXAMPLE 36

FIG. 39. Structure of a typical phosphatidylcholine lipid.

### DETAILED DESCRIPTION OF THE INVENTION

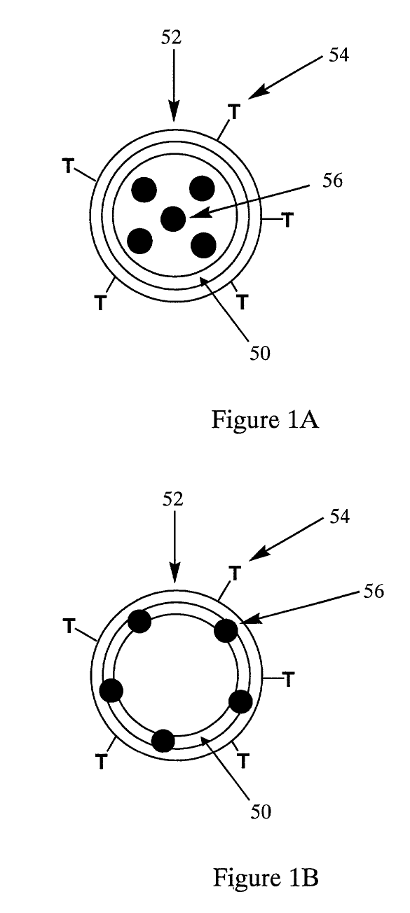

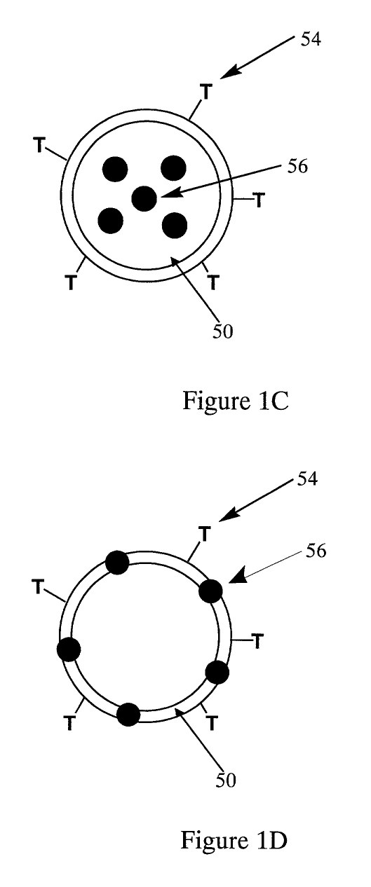

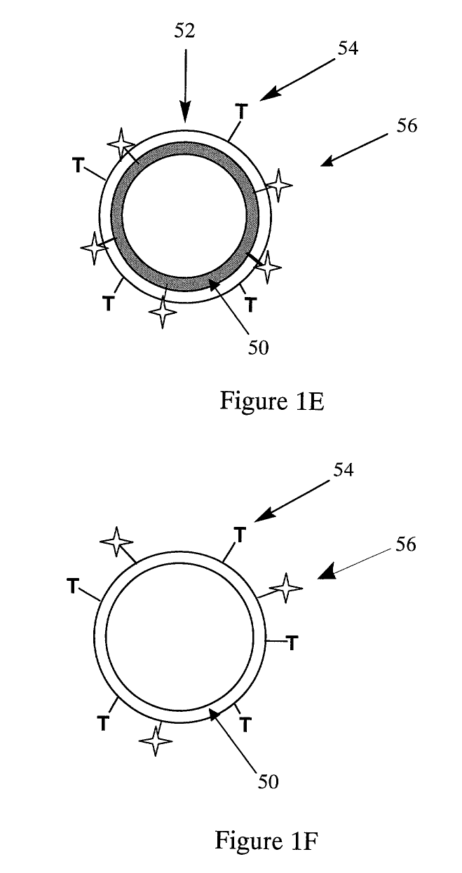

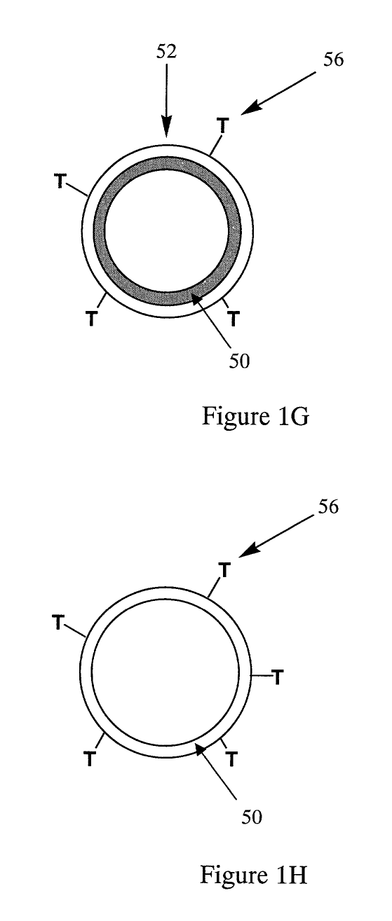

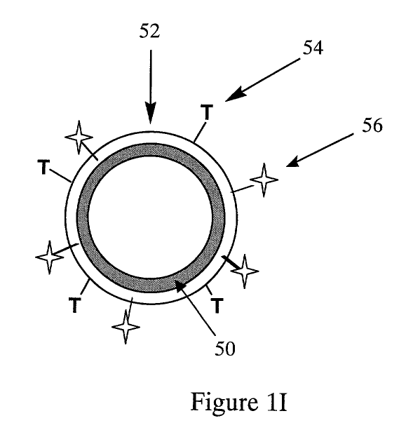

The present invention is directed toward novel targeting molecules which bind specifically and with high avidity to biological targets and methods for their preparation. This invention relates to stabilized therapeutic and imaging agents, examples of which are shown schematically in FIGS. 1A-1I which are comprised of a linking carrier, 50, a stabilizing agent, 52, a targeting entity 54, and/or a therapeutic or treatment entity, 56. As depicted in FIGS. 1A and 1B, the targeting and/or therapeutic entities may be associated with the lipid construct or the stabilizing entity. FIGS. 1A, 1B, 1C, and 1D show examples comprise both a therapeutic or targeting agent, but the agents of the invention may contain a therapeutic entity, a targeting entity, or both. Additionally, the therapeutic entity may be encapsulated within the lipid construct, or may be associated with the surface of the lipid construct or stabilizing agent. It is to be noted that the term “a” or “an” entity refers to one or more of that entity; for example, a therapeutic entity refers to one or more therapeutic entities or at least one therapeutic entity. As such, the terms “a” (or “an”), “one or more” and “at least one” can be used interchangeably herein. It is also to be noted that the terms “comprising,” “including,” and “having” can be used interchangeably.

These multivalent agents exhibit high avidity for their targets, and demonstrate up to 200-fold increase in their capacity to block cell adhesion when compared to the monomeric ligands and accumulate in vivo in tumors in a mouse melanoma model. The targeted agents of the present invention comprise more than one targeting entity. In some embodiments, the targeted agents comprise three or more targeting entities. In other embodiments, the targeted agents comprise ten or more targeting entities. In other embodiments, the targeted agents comprise 100 or more targeting entities. In other embodiments, the targeted agents comprise 1000 or more targeting entities. Examples are provided herein describing the preparation of such multivalent targeting agents, including agents comprising 0.1-30 mol % of the targeting entity.

More particularly, this invention relates to therapeutic and imaging agents which are comprised of a lipid construct, more than one targeting entity, and a therapeutic or imaging entity.

Linking Carriers

The term “linking carrier” refers to any entity which A) serves to link the therapeutic entity and the targeting entity, and B) confers additional advantageous properties to the vascular-targeted therapeutic agents other than merely keeping the therapeutic entity and the targeting entity in close proximity. Examples of these additional advantages include, but are not limited to: 1) multivalency, which is defined as the ability to attach either i) multiple therapeutic entities to the vascular-targeted therapeutic agents (i.e., several units of the same therapeutic entity, or one or more units of different therapeutic entities), which increases the effective “payload” of the therapeutic entity delivered to the targeted site; ii) multiple targeting entities to the vascular-targeted therapeutic agents (i.e., one or more units of different therapeutic entities, or, preferably, several units of the same targeting entity); or iii) both items i) and ii) of this sentence; and 2) improved circulation lifetimes, which can include tuning the size of the particle to achieve a specific rate of clearance by the reticuloendothelial system. The effective payload of therapeutic entity is the number of therapeutic entities delivered to the target site per binding event of the agent to the target. The payload will depend on the particular therapeutic entity and target. In some cases the payload will be as little as about 1 molecule delivered per binding event of the agent. In the case of a metal ion, the payload can be about one to 10³ molecules delivered per binding event. It is contemplated that the payload can be as high as 10⁴ molecules delivered per binding event. The payload can vary between about 1 to about 10⁴ molecules per binding event.

Preferred linking carriers are biocompatible polymers (such as dextran) or macromolecular assemblies of biocompatible components, such as lipid constructs, dendrimers, block copolymers, and the like. Components which may be used in the preparation of macromolecular assemblies are described herein. Examples of linking carriers include, but are not limited to, liposomes, micelles, di- and tri-block copolymers, polymerized liposomes, other lipid vesicles, dendrimers, polyethylene glycol assemblies, capped polylysines, poly(hydroxybutyric acid), dextrans, and coated polymers. A preferred linking carrier is a polymerized liposome. Polymerized liposomes are described in U.S. Pat. Nos. 5,512,294 and 6,132,764. Another preferred linking carrier is a dendrimer. A “lipid construct,” as used herein, is a structure containing lipids, phospholipids, or derivatives thereof comprising a variety of different structural arrangements which lipids are known to adopt in aqueous suspension. These structures include, but are not limited to, lipid bilayer vesicles, micelles, liposomes, emulsions, lipid ribbons or sheets, and may be complexed with a variety of drugs and components which are known to be pharmaceutically acceptable. In the preferred embodiment, the lipid construct is a liposome or polymerized vesicle.

Liposomes

As used herein, lipid refers to an agent exhibiting amphipathic characteristics causing it to spontaneously adopt an organized structure in water wherein the hydrophobic portion of the molecule is sequestered away from the aqueous phase. A lipid in the sense of this invention is any substance with characteristics similar to those of fats or fatty materials. As a rule, molecules of this type possess an extended apolar region and, in the majority of cases, also a water-soluble, polar, hydrophilic group, the so-called head-group. Phospholipids are lipids which are the primary constituents of cell membranes. Typical phospholipid hydrophilic groups include phosphatidylcholine (FIG. 39) and phosphatidylethanolamine moieties, while typical hydrophobic groups include a variety of saturated and unsaturated fatty acid moieties, including diacetylenes. Mixture of a phospholipid in water causes spontaneous organization of the phospholipid molecules into a variety of characteristic phases depending on the conditions used. These include bilayer structures in which the hydrophilic groups of the phospholipids interact at the exterior of the bilayer with water, while the hydrophobic groups interact with similar groups on adjacent molecules in the interior of the bilayer. Such bilayer structures can be quite stable and form the principal basis for cell membranes.

Bilayer structures can also be formed into closed spherical shell-like structures which are called vesicles or liposomes. The liposomes employed in the present invention can be prepared using any one of a variety of conventional liposome preparatory techniques. As will be readily apparent to those skilled in the art, such conventional techniques include sonication, chelate dialysis, homogenization, solvent infusion coupled with extrusion, freeze-thaw extrusion, microemulsification, as well as others. These techniques, as well as others, are discussed, for example, in U.S. Pat. No. 4,728,578, U.K. Patent Application G.B. 2193095 A, U.S. Pat. No. 4,728,575, U.S. Pat. No. 4,737,323, International Application PCT/US85/01161, Mayer et al., Biochimica et Biophysica Acta, Vol. 858, pp. 161-168 (1986), Hope et al., Biochimica et Biophysica Acta, Vol. 812, pp. 55-65 (1985), U.S. Pat. No. 4,533,254, Mahew et al., Methods In Enzymology, Vol. 149, pp. 64-77 (1987), Mahew et al., Biochimica et Biophysica Acta, Vol. 75, pp. 169-174 (1984), and Cheng et al., Investigative Radiology, Vol. 22, pp. 47-55 (1987), and U.S. Ser. No. 428,339, filed Oct. 27, 1989. The disclosures of each of the foregoing patents, publications and patent applications are incorporated by reference herein, in their entirety. A solvent free system similar to that described in International Application PCT/US85/01161, or U.S. Ser. No. 428,339, filed Oct. 27, 1989, may be employed in preparing the liposome constructions. By following these procedures, one is able to prepare liposomes having encapsulated therein a gaseous precursor or a solid or liquid contrast enhancing agent.

The materials which may be utilized in preparing the liposomes of the present invention include any of the materials or combinations thereof known to those skilled in the art as suitable in liposome construction. The lipids used may be of either natural or synthetic origin. Such materials include, but are not limited to, lipids such as cholesterol, phosphatidylcholine, phosphatidylethanolamine, phosphatidylserine, phosphatidylglycerol, phosphatidic acid, phosphatidylinositol, lysolipids, fatty acids, sphingomyelin, glycosphingolipids, glucolipids, glycolipids, sulphatides, lipids with amide, ether, and ester-linked fatty acids, polymerizable lipids, and combinations thereof As one skilled in the art will recognize, the liposomes may be synthesized in the absence or presence of incorporated glycolipid, complex carbohydrate, protein or synthetic polymer, using conventional procedures. The surface of a liposome may also be modified with a polymer, such as, for example, with polyethylene glycol (PEG), using procedures readily apparent to those skilled in the art. Lipids may contain functional surface groups for attachment to a metal, which provides for the chelation of radioactive isotopes or other materials that serve as the therapeutic entity. Any species of lipid may be used, with the sole proviso that the lipid or combination of lipids and associated materials incorporated within the lipid matrix should form a bilayer phase under physiologically relevant conditions. As one skilled in the art will recognize, the composition of the liposomes may be altered to modulate the biodistribution and clearance properties of the resulting liposomes.

Common adjuvants include cholesterol and alpha-tocopherol, among others. The lipid constructs may be used alone or in any combination which one skilled in the art would appreciate to provide the characteristics desired for a particular application. In addition, the technical aspects of lipid construct, vesicle, and liposome formation are well known in the art and any of the methods commonly practiced in the field may be used for the present invention. The therapeutic or treatment entity may be associated with the agent by covalent or non-covalent means. As used herein, associated means attached to by covalent or noncovalent interactions.

The membrane bilayers in these structures typically encapsulate an aqueous volume, and form a permeability barrier between the encapsulated volume and the exterior solution. Lipids dispersed in aqueous solution spontaneously form bilayers with the hydrocarbon tails directed inward and the polar headgroups outward to interact with water.

Simple agitation of the mixture usually produces multilamellar vesicles (MLVs), structures with many bilayers in an onion-like form having diameters of 1-10 μm (1000-10,000 nm). Sonication of these structures, or other methods known in the art, leads to formation of unilamellar vesicles (UVs) having an average diameter of about 30-300 nm. However, the range of 50 to 200 nm is considered to be optimal from the standpoint of, e.g., maximal circulation time in vivo. The actual equilibrium diameter is largely determined by the nature of the phospholipid used and the extent of incorporation of other lipids such as cholesterol. Standard methods for the formation of liposomes are known in the art, for example, methods for the commercial production of liposomes are described in U.S. Pat. No. 4,753,788 to Ronald C. Gamble and U.S. Pat. No. 4,935,171 to Kevin R. Bracken.

Either as MLVs or UVs, liposomes have proven valuable as vehicles for drug delivery in animals and in humans. Active drugs, including small hydrophilic molecules and polypeptides, can be trapped in the aqueous core of the liposome, while hydrophobic substances can be dissolved in the liposome membrane. Other molecules, such as DNA or RNA, may be attached to the outside of the liposome for gene therapy or gene delivery applications. The liposome structure can be readily injected and form the basis for both sustained release and drug delivery to specific cell types, or parts of the body. MLVs, primarily because they are relatively large, are usually rapidly taken up by the reticuloendothelial system (the liver and spleen). The invention typically utilizes vesicles which remain in the circulatory system for hours and break down after internalization by the target cell. For these requirements the formulations preferably utilize UVs having a diameter of less than 200 nm, preferably less than 100 nm.

Polymerized Liposomes

Polymerized liposomes, also referred to herein as “polymerized vesicles” and “nanoparticles,” are self-assembled aggregates of lipid molecules which offer great versatility in particle size and surface chemistry. Polymerized liposomes are described in U.S. Pat. Nos. 5,512,294 and 6,132,764, incorporated by reference herein in their entirety. The hydrophobic tail groups of polymerizable lipids are derivatized with polymerizable groups, such as diacetylene groups, which irreversibly cross-link, or polymerize, when exposed to ultraviolet light or other radical, anionic or cationic, initiating species, while maintaining the distribution of functional groups at the surface of the liposome. The resulting polymerized liposome particle is stabilized against fusion with cell membranes or other liposomes and stabilized towards enzymatic degradation. The size of the polymerized liposomes can be controlled by extrusion or other methods known to those skilled in the art. Polymerized liposomes may be comprised of polymerizable lipids, but may also comprise saturated and non-alkyne, unsaturated lipids. The polymerized liposomes can be a mixture of lipids which provide different functional groups on the hydrophilic exposed surface. For example, some hydrophilic head groups can have functional surface groups, for example, biotin, amines, cyano, carboxylic acids, isothiocyanates, thiols, disulfides, a-halocarbonyl compounds, α,β-unsaturated carbonyl compounds and alkyl hydrazines. These groups can be used for attachment of targeting entities, such as antibodies, ligands, proteins, peptides, carbohydrates, vitamins, nucleic acids or combinations thereof for specific targeting and attachment to desired cell surface molecules, and for attachment of therapeutic entities, such as drugs, nucleic acids encoding genes with therapeutic effect or radioactive isotopes. Other head groups may have an attached or encapsulated therapeutic entity, such as, for example, antibodies, hormones and drugs for interaction with a biological site at or near the specific biological molecule to which the polymerized liposome particle attaches. Other hydrophilic head groups can have a functional surface group of diethylenetriamine pentaacetic acid, ethylenedinitrile tetraacetic acid, tetraazocyclododecane-1,4,7,10-tetraacetic acid (DOTA), porphoryin chelate and cyclohexane-1,2,-diamino-N,N′-diacetate, as well as derivatives of these compounds, for attachment to a metal, which provides for the chelation of radioactive isotopes or other materials that serve as the therapeutic entity. Examples of lipids with chelating head groups are provided in U.S. Pat. No. 5,512,294, incorporated by reference herein in its entirety.

The polymerized liposome particle can also contain groups to control nonspecific adhesion and reticuloendothelial system uptake. For example, PEGylation of liposomes has been shown to prolong circulation lifetimes; see International Patent Application WO 90/04384.

The component lipids of polymerized liposomes can be purified and characterized individually using standard, known techniques and then combined in controlled fashion to produce the final particle. The polymerized liposomes can be constructed to mimic native cell membranes or present functionality, such as ethylene glycol derivatives, that can reduce their potential immunogenicity. Additionally, the polymerized liposomes have a well-defined bilayer structure that can be characterized by known physical techniques such as transmission electron microscopy and atomic force microscopy.

Dendrimers

Another preferred linking carrier is a dendrimer. Dendrimers are polymers with well-defined branching from a central core (e.g., “starburst polymers”). In contrast to conventional polymers, dendrimers tend to be highly branched, monodisperse macromolecules, i.e., the molecular weight tends to be very well-defined instead of a range as with conventional linear or branched polymers. Dendrimers are described in U.S. Pat. Nos. 4,507,466, 4,558,120, 4,568,737, 4,587,329, 4,631,337, 4,694,064, 4,737,550, and 4,857,599, as well as numerous other patents and patent publications. Dendrimer structure, synthesis, and characteristics are reviewed in Kim and Zimmerman, “Applications of dendrimers in bio-organic chemistry,” Current Opinion In Chemical Biology (1998) 2(6):733-42; Tam and Spetzler, “Chemoselective approaches to the preparation of peptide dendrimers and branched artificial proteins using unprotected peptides as building blocks,” Biomedical Peptides, Proteins & Nucleic Acids (1995) 1(3):123-32; Frechet, “Functional polymers and dendrimers: reactivity, molecular architecture, and interfacial energy,” Science (1994) 263(5154):1710-5; Liu and Frechet, “Designing dendrimers for drug delivery,” Pharmaceutical Science and Technology Today (1999) 2(10):393401; Verprek and Jezek “Peptide and glycopeptide dendrimers. Part I,” Journal of Peptide Science (1999) 5(1):5-23; Veprek and Jezek, “Peptide and glycopeptide dendrimers. Part II,” Journal Of Peptide Science (1999) 5(5)203-20; Tomalia et al., “Starburst dendrimers: Molecular-level control of size, shape, surface chemistry, topology, and flexibility from atoms to macroscopic matter” Angewandte Chemie—International Edition in English (1990) 29(2):138-175; Bosman et al., “About dendrimers: Structure, physical properties, and applications” Chemical Reviews (1999) 99(7):1665-1688; Fischer and Vogtle, “Dendrimers: From design to application—A progress report,” Angewandte Chemie-International Edition (1999) 38(7):885905; Roovers and Comanita, “Dendrimers And Dendrimer-Polymer Hybrids,” Advances In Polymer Science (1999) 142:179-228; Smith and Diederich, “Functional Dendrimers: Unique Biological Mimics,” Chemistry-A European Journal (1998) 4(8):1353-1361; and Matthews et al., “Dendrimers—Branching out from curiosities into new technologies,” Progress In Polymer Science (1998) 23(1):1-56. The synthesis of dendrimers typically uses reiterative synthetic cycles, allowing control over the dendrimer's size, shape, surface chemistry, flexibility, and interior topology. An example of a dendrimer suitable for use as a linking entity is described in Wu et al., “Metal-Chelate-Dendrimer-Antibody Constructs for Use in Radioimmunotherapy and Imaging,” Bioorganic and Medicinal Chemistry Letters (1994) 4(3):449-454.

Dendrimers can be readily used as linking carriers by employing a variety of chemical conjugation techniques to attach the targeting entity and therapeutic entity. For example, in U.S. Pat. No. 6,020,457, which discloses a dendrimer having a disulfide (—S—S—) bond in its core, the dendrimer can be constructed by the methods described in the patent. The final external layer of the dendrimer can be capped with a reactive group such as an amine or carboxyl group. These reactive groups can then be derivatized with either targeting entities or therapeutic entities (or, in some cases, a mixture of both). The core disulfide bond can then be reduced to a thiol, and the complementary entity attached via the thiol functionality. That is, if a therapeutic entity had been attached to the external layer of the dendrimeric linking carrier, upon reduction and formation of the thiol functionality, a targeting entity can be attached via the free -SH group. One example of such targeting entity is an N-terminal-iodoacetylated peptide (the peptide may be a hormone or bioactive fragment of a larger protein), which is readily synthesized by standard solid-phase peptide techniques. The iodoacetyl group will react with the free thiol functionality, resulting in the conjugation of the therapeutic-entity-derivatized linking carrier with the targeting entity (the peptide).

Block Copolymers

A block copolymer, as used herein, is combination of two or more chains of constitutionally or configurationally different features. A block copolymer can be used as a linking carrier by employing a variety of chemical conjugation techniques to attach the targeting entity and therapeutic entity. Block copolymers include diblock, triblock, or multiblock copolymers.

The use of amphiphilic block copolymer micelles has recently been attracting much interest as a potentially effective drug carrier which is capable of solubilizing a hydrophobic drug in an aqueous environment. For example, there have been reported many studies on amphiphilic block copolymer micelles having surfactant-like properties, and particularly noteworthy are the attempts to incorporate hydrophobic drugs into block copolymer micelles stabilized due to the specific nature and properties of the copolymer. For example, EP No. 0 397 307 A2 discloses polymeric micelles of an AB type amphiphilic diblock copolymer which contains poly(ethylene oxide) as the hydrophilic component and poly(amino acids) as the hydrophobic component, wherein therapeutically active agents are chemically bonded to the hydrophobic component of the polymer. EP No. 0 583 955 A2, discloses a method for physically incorporating hydrophobic drugs into amphiphilic diblock copolymer micelles described in EP No. 0 397 307 A2. EP No. 0 552 802 A2 discloses formation of chemically fixed micelles having poly(ethylene oxide) as the hydrophilic component and poly(lactic acid) as the hydrophobic component which can be crosslinked in an aqueous phase. U.S. Pat. No. 4,745,160 discloses a pharmaceutically or veterinary acceptable amphiphilic, non-cross linked linear, branched or graft block copolymer having polyethylene glycol as the hydrophilic component and poly(D-, L- and DL-lactic acids) as the hydrophobic components. U.S. Pat. No. 5,543,158 discloses nanoparticle or microparticle formed of a block copolymer consisting essentially of poly(alkylene glycol) and a biodegradable polymer, poly(lactic acid). In the nanoparticle or microparticle, the biodegradable moieties of the copolymer are in the core of the nanoparticle or microparticle and the poly(alkylene glycol) moieties are on the surface of the nanoparticle or microparticle in an amount effective to decrease uptake of the nanoparticle or microparticle by the reticuloendothelial system. U.S. Pat. No. 6,007,845 describes a multiblock copolymer-based composition prepared by covalently linking a multifunctional compound with one or more hydrophobic polymers and one or more hydrophilic polymers, and containing a biologically active material. U.S. Pat. No. 5,543,158 provides for block copolymer bas-ed particles that are not rapidly cleared from the blood stream by the macrophages of the reticuloendothelial system, and that can be modified as necessary to achieve variable release rates or to target specific cells or organs as desired. The particles have a biodegradable solid core containing a biologically active material and poly(alkylene glycol) moieties on the surface. The terminal hydroxyl group of the poly(alkylene glycol) can be used to covalently attach onto the surface antibodies targeted to specific cells or organs, or molecules affecting the charge, lipophilicity or hydrophilicity of the particle.

Examples of biocompatible polymers suitable for use as linking carrier block copolymers in the present inveintion are poly(ethylene-covinyl acetate), and silicone rubber cross linked to poly (dimethyl siloxan sulfoxide) and derivatives thereof, polylactic acid, polyglycolic acid or polycaprolactone and their associated copolymers, e.g. poly (lactide-co-glycolide) at all lactide to glycolide ratios, and both L-lactide or D,L lactide. Additional hydrophilic polymers include polypyrrolidone, poly(amino acids), including short non-toxic and non-immunogenic proteins and peptides such as human albumin, fibrin, gelatin and fragments thereof, dextrans, and poly(vinyl alcohol). Other materials include a Pluronic™ F68 (BASF Corporation), a copolymer of polyoxyethylene and polyoxypropylene, which is approved by the U.S. Food and Drug Administration (FDA). Other hydrophobic polymers can be polyanhydrides, polydioxanones, polyphosphazenes, polymers of α-hydroxy carboxylic acids, polyhydroxybutyric acid, polyorthoesters, polycaprolactone, polyphosphates, or copolymers prepared from the monomers of these polymers can be used to form the multiblock copolymers described herein. The variety of materials that can be used to prepare the block copolymers forming the particles significantly increases the diversity of release rate and profile of release that can be accomplished in vivo.

In a preferred embodiment, a polyester of poly(lactic-co-glycolic)acid (PLGA) is used as a hydrophobic erodible polymer bound to the multifunctional compound. These polymers are approved for parenteral administration by the FDA.

The block copolmers of the present invention are preferably composed of a polymeric-backbone having an interactive region for physically cross-linking with other entities, including targeting entities, therapeutic entities, or other polymers. Preferably, the backbone of the polymer comprises a plurality of interactive regions. The functional groups encompass conjugatable groups such as for example amines, hydroxyls, carbonyls, thiols, and carboxylic acids for covalently bonding of other bioactive molecules to the surface of the particle, as described in mre detail below. The linkages formed following conjugation of the bioactive molecules to the conjugatable groups include amides, esters, and thioethers. Examples of copolymers which have conjugatable functional groups include (poly) lysine, acetylated poly (lysine); poly (glutamic acid, and poly(oxyethylene)-poly (oxyproplene) copolymers.

Therapeutic Entities

The term “therapeutic entity” refers to any molecule, molecular assembly or macromolecule that has a therapeutic effect in a treated subject, where the treated subject is an animal, preferably a mammal, more preferably a human. The term “therapeutic effect” refers to an effect which reverses a disease state, arrests a disease state, slows the progression of a disease state, ameliorates a disease state, relieves symptoms of a disease state, or has other beneficial consequences for the treated subject. Therapeutic entities include, but are not limited to, drugs, including antibiotics, drugs such as doxorubicin, paclitaxel, and other chemotherapy agents including camptothecin and topotecan; small molecule therapeutic drugs, toxins such as ricin; radioactive isotopes; genes encoding proteins that exhibit cell toxicity, and prodrugs (drugs which are introduced into the body in inactive form and which are activated in situ).

Radioisotopes useful as therapeutic entities are described in Kairemo, et al., Acta Oncol. 35:343-55 (1996), and include Y-90, 1-123, I-125, 1-131, Bi-213, At-211, Cu-67, Sc-47, Ga-67, Rh-105, Pr-142, Nd-147, Pm-151, Sm-153, Ho-166, Gd-159, Th-161, Eu-152, Er-171, Re-186, and Re-188.

Additional therapeutic agents include but are not limited to cytotoxic or cytostatic agents that target growth factors, cell cycle modulators, Bcl-2, TNF-α receptor, cyclin-dependent kinases, the Ras pathway, the EGFR pathway, and other relevant cellular pathways, proteins involved in multi-drug resistance including p-glycoprotein, tubulins, DNA, RNA, topoisomerases, telomerases, and kinases, and enzymes involved in DNA methylation. These therapeutic agents may be alkylating agents, cisplatinum and derivatives, pyrimidine and purine analogues, topoisomerase inhibitors, microtuble-targeting agents, estrogen derivatives, androgen derivatives, interferons, intercalating agents, and MDR inhibitors, for example. Specific agents include tubulin-binding molecules vincristine, vinblastine, vindesine, and vinorelbine.

In another preferred embodiment, the therapeutic entity is an intracellular kinase inhibitor such as AG1433 or SU1498 (FIGS. 35A and 35B, respectively) and the target is Flk-1/KDR. It should be noted that therapeutic entities such as AG1433 or SU1498 could also be classified as targeting entities; likewise, some targeting entities may also act as therapeutic entities.

In preferred embodiments of the present invention, the therapeutic entity is encapsulated within a liposome or polymerized vesicle or associated by covalent or non-covalent means with the linking carrier or macromolecular assembly. Preferably, these agents are encapsulated in amounts such that the dose of targeted therapeutic agents is effective to treat the disease.

In other preferred embodiments, the therapeutic entity is associated with the surface of a liposome or polymerized vesicle.

Stabilizing Entities

The agents of the present invention preferably contain a stabilizing entity. As used herein, “stabilizing” refers to the ability to imparts additional advantages to the therapeutic or imaging agent, for example, physical stability, i.e., longer half-life, colloidal stability, and/or capacity for multivalency; that is, increased payload capacity due to numerous sites for attachment of targeting agents. As used herein, “stabilizing entity” refers to a macromolecule or polymer, which may optionally contain chemical functionality for the association of the stabilizing entity to the surface of the vesicle, and/or for subsequent association of therapeutic entities or targeting agents. The polymer should be biocompatible with aqueous solutions. Polymers useful to stabilize the liposomes of the present invention may be of natural, semi-synthetic (modified natural) or synthetic origin. A number of stabilizing entities which may be employed in the present invention are available, including xanthan gum, acacia, agar, agarose, alginic acid, alginate, sodium alginate, carrageenan, gelatin, guar gum, tragacanth, locust bean, bassorin, karaya, gum arabic, pectin, casein, bentonite, unpurified bentonite, purified bentonite, bentonite magma, and colloidal bentonite.

Other natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrose, dextrin, glucose, polyglucose, polydextrose, pustulan, chitin, agarose, keratin, chondroitin, dermatan, hyaluronic acid, alginic acid, xanthin gum, starch and various other natural homopolyner or heteropolymers, such as those containing one or more of the following aldoses, ketoses, acids or amines: erythrose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Other suitable polymers include proteins, such as albumin, polyalginates, and polylactide-glycolide copolymers, cellulose, cellulose (microcrystalline), methylcellulose, hydroxyethylcellulose, hydroxypropylcellulose, hydroxypropylmethylcellulose, carboxymethylcellulose, and calcium carboxymethylcellulose.

Exemplary semi-synthetic polymers include carboxymethylcellulose, sodium carboxymethylcellulose, carboxymethylcellulose sodium 12, hydroxymethylcellulose, hydroxypropylmethylcellulose, methylcellulose, and methoxycellulose. Other semi-synthetic polymers suitable for use in the present invention include carboxydextran, aminodextran, dextran aldehyde, chitosan, and carboxymethyl chitosan.

Exemplary synthetic polymers include poly(ethylene imine) and derivatives, polyphosphazenes, hydroxyapatites, fluoroapatite polymers, polyethylenes (such as, for example, polyethylene glycol, the class of compounds referred to as Pluronics®, commercially available from BASF, (Parsippany, N.J.), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof, polysorbate, carbomer 934P, magnesium aluminum silicate, aluminum monostearate, polyethylene oxide, polyvinylalcohol, povidone, polyethylene glycol, and propylene glycol. Methods for the preparation of vesicles which employ polymers to stabilize vesicle compositions will be readily apparent to one skilled in the art, in view of the present disclosure, when coupled with information known in the art, such as that described and referred to in Unger, U.S. Pat. No. 5,205,290, the disclosure of which is hereby incorporated by reference herein in its entirety.

In a preferred embodiment, the stabilizing entity is dextran. In another preferred embodiment, the stabilizing entity is a modified dextran, such as amino dextran. In a further preferred embodiment, the stabilizing entity is poly(ethylene imine) (PEI). Without being bound by theory, it is believed that dextran may increase circulation times of liposomes in a manner similar to PEG. Additionally, each polymer chain (i.e. aminodextran or succinylated aminodextran) contains numerous sites for attachment of targeting agents, providing the ability to increase the payload of the entire lipid construct. This ability to increase the payload differentiates the stabilizing agents of the present invention from PEG. For PEG there is only one site of attachment, thus the targeting agent loading capacity for PEG (with a single site for attachment per chain) is limited relative to a polymer system with multiple sites for attachment.

In other preferred embodiments, the following polymers and their derivatives are used. Poly(galacturonic acid), poly(L-glutamic acid), poly(L-glutamic acid-L-tyrosine), poly[R)-3-hydroxybutyric acid], poly(inosinic acid potassium salt), poly(L-lysine), poly(acrylic acid), poly(ethanolsulfonic acid sodium salt), poly(methylhydrosiloxane), poly(vinyl alcohol), poly(vinylpolypyrrolidone), poly(vinylpyrrolidone), poly(glycolide), poly(lactide), poly(lactide-co-glycolide), and hyaluronic acid. In other preferred embodiments, copolymers including a monomer having at least one reactive site, and preferably multiple reactive sites, for the attachment of the copolymer to the vesicle or other molecule.

In some embodiments, the polymer may act as a hetero- or homobifunctional linking agent for the attachment of targeting agents, therapeutic entities, proteins or chelators such as DTPA and its derivatives.

In one embodiment, the stabilizing entity is associated with the vesicle by covalent means. In another embodiment, the stabilizing entity is associated with the vesicle by non-covalent means. Covalent means for attaching the targeting entity with the liposome are known in the art and described in the EXAMPLES section.

Noncovalent means for attaching the targeting entity with the liposome include but are not limited to attachment via ionic, hydrogen-bonding interactions, including those mediated by water molecules or other solvents, hyrdophobic interactions, or any combination of these.

In a preferred embodiment, the stabilizing agent forms a coating on the liposome.

Targeting Entities

The term “targeting entity” refers to a molecule, macromolecule, or molecular assembly which binds specifically to a biological target. Examples of targeting entities include, but are not limited to, antibodies (including antibody fragments and other antibody-derived molecules which retain specific binding, such as Fab, F(ab′)2, Fv, and scFv derived from antibodies); receptor-binding ligands, such as hormones or other molecules that bind specifically to a receptor; cytokines, which are polypeptides that affect cell function and modulate interactions between cells associated with immune, inflammatory or hematopoietic responses; molecules that bind to enzymes, such as enzyme inhibitors; nucleic acid ligands or aptamers, and one or more members of a specific binding interaction such as biotin or iminobiotin and avidin or streptavidin. Preferred targeting entities are molecules which specifically bind to receptors or antigens found on vascular cells. More preferred are molecules which specifically bind to receptors, antigens or markers found on cells of angiogenic neovasculature or receptors, antigens or markers associated with tumor vasculature. The receptors, antigens or markers associated with tumor vasculature can be expressed on cells of vessels which penetrate or are located within the tumor, or which are confined to the inner or outer periphery of the tumor. In one embodiment, the invention takes advantage of pre-existing or induced leakage from the tumor vascular bed; in this embodiment, tumor cell antigens can also be directly targeted with agents that pass from the circulation into the tumor interstitial volume.

Other targeting entities target endothelial receptors, tissue or other targets accessible through a body fluid or receptors or other targets upregulated in a tissue or cell adjacent to or in a bodily fluid. For example, targeting entities attached to carriers designed to deliver drugs to the eye can be injected into the vitreous, choroid, or sclera; or targeting agents attached to carriers designed to deliver drugs to the joint can be injected into the synovial fluid.

The targeting entity may have other effects, including therapeutic effects, in addition to specifically binding to a target. For example, the targeting entity may modulate the function of an enzyme target. By “modulate the function” it is meant altering when compared to not adding the targeting entity. In most cases, a preferred form of modulation of function is inhibition. Examples of targeting agents which may have other functions or effects are described herein. Other targeting entities that fall into this category include Combrestastatin A4 Prodrug (CA4P) (Oxigene/BMS) which may be used as a vascular targeting agent that also acts as an anti-angiogenesis agent, and Cidecin (Cubist Pharm/Emisphere) a cyclic lipopeptide used as a bactericidal and anti-inflammatory agent.

Targeting entities attached to the polymerized liposomes, or linking carriers of the invention include, but are not limited to, small molecule ligands, such as carbohydrates, and compounds such as those disclosed in U.S. Pat. No. 5,792,783 (small molecule ligands are defined herein as organic molecules with a molecular weight of about 5000 daltons or less); proteins, such as antibodies and growth factors; peptides, such as RGD-containing peptides (e.g. those described in U.S. Pat. No. 5,866,540), bombesin or gastrin-releasing peptide, peptides selected by phage-display techniques such as those described in U.S. Pat. No. 5,403,484, and peptides designed de novo to be complementary to tumor-expressed receptors; antigenic determinants; or other receptor targeting groups.

These targeting entities can be used to control the biodistribution, non-specific adhesion, and blood pool half-life of the lipid constructs. For example, β-D-lactose targets the asialoglycoprotein (ASG) found in liver cells which are in contact with the circulating blood pool. Glycolipids can be derivatized for use as targeting entities by converting the commercially available lipid (DAGPE) or PEG-PDA amines into glycolipids.

In some embodiments, the targeting entity targets the liposomes to a cell surface. Delivery of the therapeutic or imaging agent can occur through endocytosis of the liposomes. Such deliveries are known in the art. See, for example, Mastrobattista, et al., Immunoliposomes for the Targeted Delivery of Antitumor Drugs, Adv. Drug Del. Rev. (1999) 40:103-27.

In one embodiment, the attachment is by covalent means. In another embodiment, the attachment is by non-covalent means. For example, antibody targeting entities may be attached by a biotin-avidin biotinylated antibody sandwich to allow a variety of commercially available biotinylated antibodies to be used on the coated polymerized liposome.

In a preferred embodiment, the targeting entity is a small molecule ligand peptidomimetic which binds to chemokine receptors CCR4 and CCR5, VCAM, EGFR, FGFR, matrix metalloproteases (MMPs) including surface associated MMPs, PDGFR, P- and E-selectins, pleiotropin, Flk-1/KDR, Flt-1, Tek, Tie, neuropilin-1, endoglin, endosialin, Axl, αᵥβ₃, αᵥβ₅, α₅β₁, α₄β₁, α₁β₁, α₂β₂, or prostate specific membrane antigen (PSMA). Additional targets are described by E. Ruoslahti in Nature Reviews: Cancer, 2, 83-90 (2002). Further targets include the CD family of cell surface antigens including CD1 through CD178, and any target that is accessible to the targeting agent by administration to a patient including extracellular matrix components that are exposed in diseased tissue but less so in normal tissue.

Examples of targeting entities which may be used in the targeted agents of the present invention include, but are not limited to Conivaptan (Yamanouchi Pharm.), a V1 & V2 vasopressin receptor antagonist; GBC-590 (Abbott/GlycoGenesys), a lectin inhibitor useful in prevention of metastasis; Veletri (Actelion), an endothelin antagonist (tesosentan); VLA-4 Antagonist (Aventis) an agent with potential for treating rheumatoid arthritis, multiple sclerosis, cardiovascular disease and other conditions; Campath (Berlex/Millenium), a monoclonal antibody specific for CD52+ malignant lymphocytes; Tracleer (Actelion), an endothelin antagonist (bosentan) approved for the treatment of pulmonary arterial hypotension; and Natrecor (Scios), a natriuretic peptide that binds to vascular smooth muscle cells and endothelial cells.

In a preferred embodiment, the targeting entity is an integrin-specific molecule. The integrin specific molecule may be an RDG peptide or derivative thereor. Other integrin-specific molecules are described, for instance, in U.S. Pat. No. 5,561,148; U.S. Pat. No. 6,204,280, International Publication No. WO 01/14338, and International Publication No. WO 01/14337. In a particularly preferred embodiment, the targeting entity is compound 10, 3-{4-[2-(3,4,5,6-tetrahydropyrimidin-2-ylamino)-ethyloxy]-benzoylamino}-2(S)-benzene-sulfonyl-aminopropionic acid, and the target is αᵥβ₃. In another embodiment, the integrin-specific molecule is Cilengitide. In another particularly preferred embodiment, the targeting entity is a protease inhibitor such as N-acetyl-Leu-Val-Lys-aldehyde (Bachem N-1380) or Gly-Phe-Gly-aldehyde semicarbazone (Bachem C-3085) and the target is papain or cathespin B.

An antitumor agent can be a conventional antitumor therapy, such as cisplatin; antibodies directed against tumor markers, such as anti-Her2/neu antibodies (e.g., Herceptin); or tripartite agents, such as those described herein for vascular-targeted therapeutic agents, but targeted against the tumor cell rather than the vasculature. A summary of monoclonal antibodies directed against various tumor markers is given in Table I of U.S. Pat. No. 6,093,399, hereby incorporated by reference herein in its entirety. In general, when the vascular-targeted therapy agent compromises vascular integrity in the area of the tumor, the effectiveness of any drug which operates directly on the tumor cells can be enhanced.

In one embodiment of the invention, a vascular-targeted therapeutic agent is combined with an agent targeted directly towards tumor cells. This embodiment takes advantage of the fact that the neovasculature surrounding tumors is often highly permeable or “leaky,” allowing direct passage of materials from the bloodstream into the interstitial space surrounding the tumor. Alternatively, the targeted therapeutic agent itself can induce permeability in the tumor vasculature. For example, when the agent carries a radioactive therapeutic entity, upon binding to the vascular tissue and irradiating that tissue, cell death of the vascular epithelium will follow and the integrity of the vasculature will be compromised.

Accordingly, in one embodiment, the vascular-targeted therapeutic agent has two targeting entities: a targeting entity directed towards a vascular marker, and a targeting entity directed towards a tumor cell marker. In another embodiment, an antitumor agent is administered with the vascular-targeted therapy agent. The antitumor agent can be administered simultaneously with the vascular-targeted therapy agent, or subsequent to administration of the vascular-targeted therapy agent. In particular, when the vascular-targeted therapy agent is relied upon to compromise vascular integrity in the area of the tumor, administration of the antitumor agent is preferably done at the point of maximum damage to the tumor vasculature.

The size of the vesicles can be adjusted for the particular intended end use including, for example, diagnostic and/or therapeutic use. As the size of the linking carrier can be manipulated readily, the overall size of the vascular-targeted therapeutic agents can be adapted for optimum passage of the particles through the permeable (“leaky”) vasculature at the site of pathology, as long as the agent retains sufficient size to maintain its desired properties (e.g., circulation lifetime, multivalency). Accordingly, the particles can be sized at 30, 50, 100, 150, 200, 250, 300 or 350 nm in size, as desired. In addition, the size of the particles can be chosen so as to permit a first administration of particles of a size that cannot pass through the permeable vasculature, followed by one or more additional administrations of particles of a size that can pass through the permeable vasculature. The size of the vesicles may preferably range from about 30 nanometers (nm) to about 400 nm in diameter, and all combinations and subcombinations of ranges therein. More preferably, the vesicles have diameters of from about 10 nm to about 500 nm, with diameters from about 40 nm to about 120 nm being even more preferred. In connection with particular uses, for example, intravascular use, including magnetic resonance imaging of the vasculature, it may be preferred that the vesicles be no larger than about 500 nm in diameter, with smaller vesicles being preferred, for example, vesicles of no larger than about 100 nm in diameter. It is contemplated that these smaller vesicles may perfuse small vascular channels, such as the microvasculature, while at the same time providing enough space or room within the vascular channel to permit red blood cells to slide past the vesicles.

Further therapeutics contemplated for use in the invention include but are not limited to AGI-1067 (Atherogenics), for the treatment of restenosis, nystatin, an antifungal agent, and Gleevec, which blocks Bcr-Abl intracellular protein in white blood cells.

While one major focus of the invention is the use of vascular-targeted therapy agent against the vasculature of tumors in order to treat cancer, the agents of the invention can be used in any disease where neovascularization or other aberrant vascular growth accompanies or contributes to pathology. Diseases associated with neovascular growth include, but are not limited to, solid tumors; blood-borne tumors such as leukemias; tumor metastasis; benign tumors, for example hemangiomas, acoustic neuromas, neurofibromas, trachomas, and pyogenic granulomas; rheumatoid arthritis; psoriasis; chronic inflammation; ocular angiogenic diseases, for example, diabetic retinopathy, retinopathy of prematurity, macular degeneration, corneal graft rejection, neovascular glaucoma, retrolental fibroplasia, rubeosis; arteriovenous malformations; ischemic limb angiogenesis; Osler-Webber Syndrome; myocardial angiogenesis; plaque neovascularization; telangiectasia; hemophiliac joints; angiofibroma; and wound granulation. Diseases of excessive or abnormal stimulation of endothelial cells include, but are not limited to, intestinal adhesions, atherosclerosis, restenosis, scleroderma, and hypertrophic scars, i.e., keloids.

Differing administration vehicles, dosages, and routes of administration can be determined for optimal administration of the agents; for example, injection near the site of a tumor may be preferable for treating solid tumors. Therapy of these disease states can also take advantage of the permeability of the neovasulature at the site of the pathology, as discussed above, in order to specifically deliver the vascular-targeted therapeutic agents to the interstitial space at the site of pathology.

Targeted Multivalent Agents

The linking carrier can be coupled to the targeting entity and the therapeutic entity by a variety of methods, depending on the specific chemistry involved. The coupling can be covalent or non-covalent. A variety of methods suitable for coupling of the targeting entity and the therapeutic entity to the linking carrier can be found in Hermanson, “Bioconjugate Techniques”, Academic Press: New York, 1996; and in “Chemistry of Protein Conjugation and Cross-linking” by S. S. Wong, CRC Press, 1993. Specific coupling methods include, but are not limited to, the use of bifunctional linkers, carbodiimide condensation, disulfide bond formation, and use of a specific binding pair where one member of the pair is on the linking carrier and another member of the pair is on the therapeutic or targeting entity, e.g. a biotin-avidin interaction.

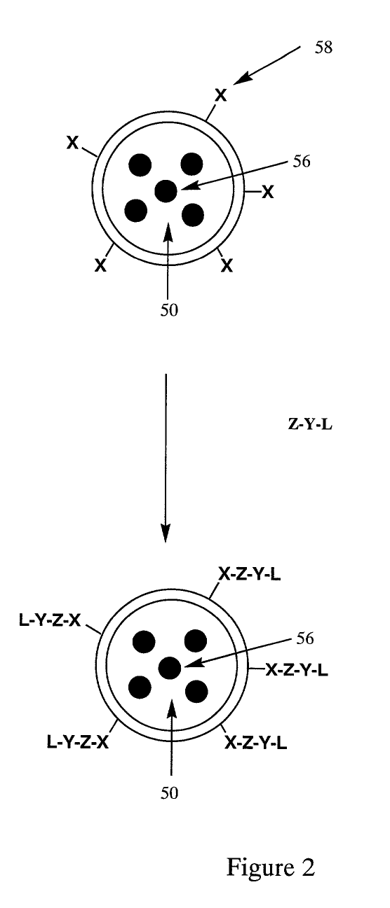

A schematic of the coupling of a ligand Z-Y-L where Z is a chemically reactive moiety covalently attached to a spacer Y that is covalently attached to ligand L is shown in FIG. 2. This conjugation may require an activating agent.

Generally, prior to forming the linkage between the targeting entity and the lipid, linking carrier, and/or optionally, the spacer group, at least one of the chemical functionalities will be activated. One skilled in the art will appreciate that a variety of chemical functionalities, including hydroxy, amino, and carboxy groups, can be activated using a variety of standard methods and conditions. For example, a hydroxyl group of the ligand or lipid can be activated through treatment with phosgene to form the corresponding chloroformate. In addition, if the hydroxyl functionality is part of a sugar residue, then the hydroxyl group can be activated through reaction with di-(n-butyl)tin oxide to form a tin complex.

Carboxy groups may be activated by conversion to the corresponding acyl halide. This reaction may be performed under a variety of conditions as illustrated in Jerry March, Advanced Organic Chemistry: Reactions, Mechanisms, and Structure, Fourth Ed., at 388-89. In one embodiment, the acyl halide is prepared through the reaction of the carboxy containing group with oxalyl chloride.

Typically, the lipid or linking carrier is linked covalently to a targeting entity using standard chemical techniques through their respective chemical functionalities. Optionally, the targeting entity can be coupled to the lipid or liking carrier through one or more spacer groups. The spacer groups can be equivalent or different when used in combination.

The lipid-targeting agent complex is prepared by linking a lipid to a targeting entity (or optionally to a spacer group which has been or will be attached to a targeting group) via their respective chemical functionalities. Preferably, the lipid (e.g., chemical functionality 1) is joined to the targeting entity, optionally via a spacer group, (e.g., chemical functionality 2) via the linkages shown in Table 3. Those of skill in the art will recognize that one can first attach the spacer either to the targeting agent or to the lipid. The chemical functionalities shown in Table 3 can be present on the targeting entity, spacer, or lipid, depending on the synthesis scheme employed.

TABLE 3

| Chemical | Chemical | |
|---|---|---|
| Functionality 1 | Functionality 2 | Linkage |
| Hydroxy | Carboxy | Ester |
| | Hydroxy | Carbonate |
| | Amine | Carbamate |
| | SO₃ | Sulfate |
| | | Phosphate |
| | Carboxy | Acyloxyalkyl |
| | | ether |
| | Ketone | Ketal |
| | Aldehyde | Acetal |
| | Hydroxy | Anhydride |
| Mercapto | Mercapto | Disulfide |
| | Carboxy | Acyloxyalkyl |
| | | thioether |
| | Carboxy | Thioester |
| Carboxy | Amino | Amide |
| | Mercapto | Thioester |
| | Carboxy | Acyloxyalkyl |
| | | ester |
| | Carboxy | Acyloxyalkyl |
| | | amide |
| | Amino | Acyloxyalkoxy |
| | | carbonyl |
| | Carboxy | Anhydride |
| | Carboxy | N-acylamide |
| | Hydroxy | Ester |
| | Hydroxy | Hydroxy- |
| | | methyl |
| | | ketone ester |
| | Hydroxy | Alkoxy- |
| | | carbonyl |
| | | oxyalkyl |
| Amino | Carboxy | Acyloxyalkyl |
| | | amine |
| | Carboxy | Acyloxyalkyl |
| | | amide |
| | Amino | Urea |
| | Carboxy | Amide |
| | Carboxy | Acyloxyalkoxy |
| | | carbonyl |
| | Amide | N-Mannich |
| | | base |
| | Carboxy | Acyloxyalkyl |
| | | carbamate |
| Phosphate | Hydroxy | Phosphate |
| | Oxygen | ester |
| | Amine | Phosphor- |
| | | amidate |
| | Mercapto | Thiophosphate |
| | | ester |
| Ketone | Carboxy | Enol ester |
| Sulfonamide | Carboxy | Acyloxyalkyl |
| | | sulfonamide |
| | Ester | N-sulfonyl- |
| | | imidate |

One skilled in the art will readily appreciate that many of these linkages may be produced in a variety of ways and using a variety of conditions. For the preparation of esters, see, e.g., March, ibid., at 1157; for thioesters, see March, supra at 362-363, 491, 720-722, 829, 941, and 1172; for carbonates, see March, supra at 346-347; for carbamates, see March, supra at 1156-57; for amides, see March supra at 1152; for ureas and thioureas, see March supra at 1174; for acetals and ketals, see Greene et al. supra 178-210 and March supra at 1146; for acyloxyalkyl derivatives, see Prodrugs: Topical and Ocular Drug Delivery, K. B. Sloan, ed., Marcel Dekker, Inc., New York, 1992; for enol esters, see March supra at 1160; for N-sulfonylimidates, see Bundgaard et al., (1988) J. Med. Chem., 31:2066; for anhydrides, see March supra at 355-56, 636-37, 990-91, and 1154; for N-acylamides, see March supra at 379; for N-Mannich bases, see March supra at 800-02, and 828; for hydroxymethyl ketone esters, see Petracek et al. (1987) Annals NY Acad. Sci., 507:353-54; and for disulfides, see March supra at 1160.

A variety of ketal type linkages may be produced. Ketal type linkages that may be produced in the pharmaceutical agent-chemical modifier complexes of the present invention include, but are not limited to, imidazolidin-4-ones, see Prodrugs, supra; oxazolin-5-ones, see Greene et al. supra at 358; dioxolan-4-one, see Schwenker et al. (1991) Arch. Pharm. (Weinheim) 324:439; spirothiazolidines, see Bodor et al. (1982) Int. J. Pharm., 10:307 and Greene et al. supra at 219 and 292; and oxazolidines, see March supra at 87 and Greene et al. supra at 217-218 and 266-267.

In a preferred embodiment, the targeting entity is attached to a carboxyl head group on the lipid. In another preferred embodiment, the targeting entity is attached to a maleimide or the alpha-methyl group of an acetamide.

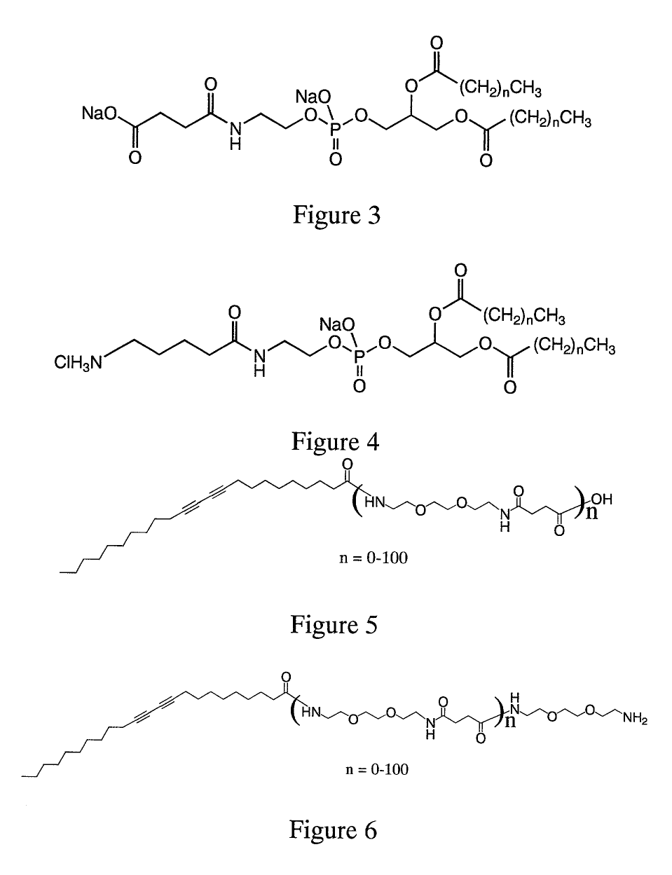

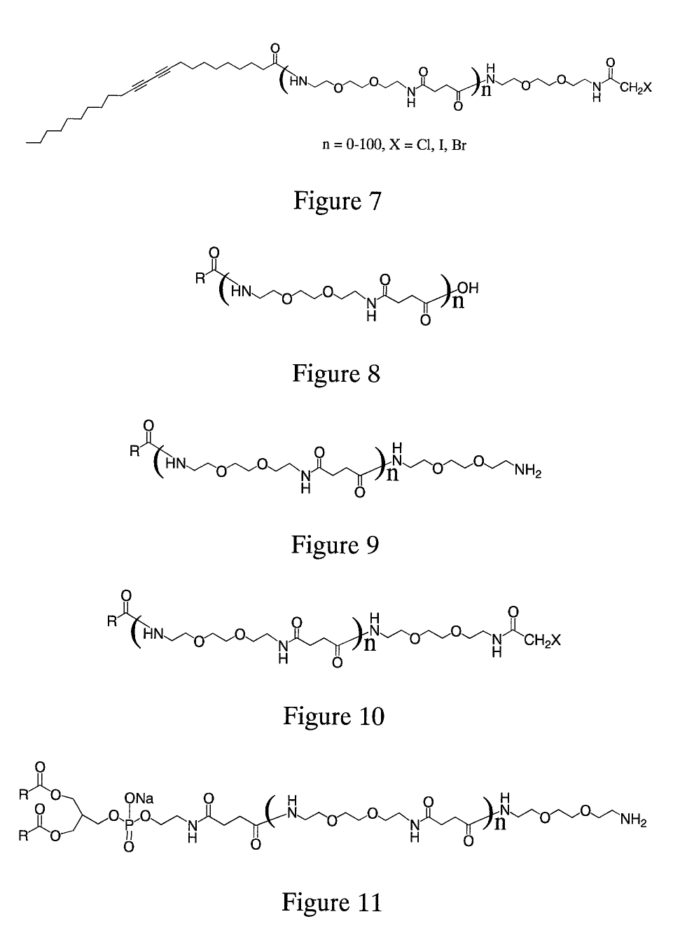

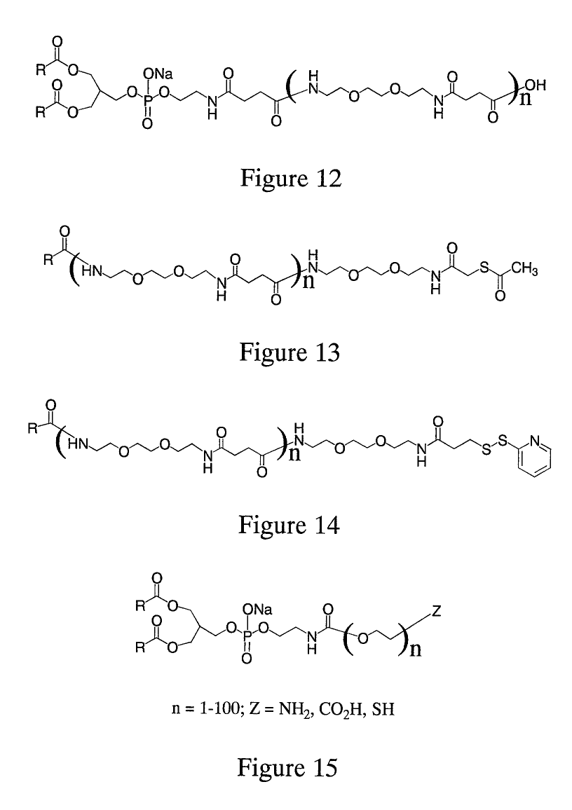

Exemplary lipids with a variety of functionalites for linking a lipid to a targeting entity or therapeutic entity are shown in FIGS. 3-15. Additional linkages and functionalities, for example, for the attachment of nucleic acids, are desrcribed in Hale, et al., U.S. Pat. No. 5,607,691.

One or more spacer groups optionally may be introduced between the lipid and the targeting entity. Spacer groups typically contain two chemical functionalities and, typically do not carry a charge. Typically, one chemical functionality of the spacer group bonds to a chemical functionality of the lipid, while the other chemical functionality of the spacer group is used to bond to a chemical functionality of the targeting entity. Examples of chemical functionalities of spacer groups include hydroxy, mercapto, carbonyl, carboxy, amino, ketone, and mercapto groups. Spacer groups may also be used in combination. When a combination of spacer groups is used, the spacer groups may be different or equivalent.

Preferred spacer groups include 6-aminohexanol, 6-mercaptohexanol, 10-hydroxydecanoic acid, glycine and other amino acids, 1,6-hexanediol, beta-alanine, 2-aminoethanol, cysteamine (2-aminoethanethiol), 5-aminopentanoic acid, 6-aminohexanoic acid, 3-maleimidobenzoic acid, phthalide, alpha-substituted phthalides, the carbonyl group, aminal esters, and the like. Particularly preferred spacer groups are also depicted schematically in FIGS. 3-15, and include polyethylene glycol, and ethylene glycol derivatives with terminal amino groups.

The spacer can serve to introduce additional molecular mass and chemical functionality into the linking carrier-targeting entity complex. Generally, the additional mass and functionality will affect the serum half-life and other properties of the pharmaceutical agent-chemical modifier complex. Thus, through careful selection of spacer groups, linking carrier-targeting entity complexes with a range of serum half-lives can be produced.

In addition, the nature of the linkage used to couple the spacer group to the chemical modifier or pharmaceutical agent may affect the serum half-life.

Although discussion has thus far focused on the coupling of a single type of targeting entity to a linking carrier, in some embodiments, other entities can be coupled to the linking carrier or the linking carrier-targeting entity complex. Other entities which can be covalently bound to the linking carrier-targeting entity complex (optionally via a spacer group), will serve to affect or modify a chemical, physical, or biological property of the complex, including providing a means for detection, for increasing the excretion half-life of the complex, for decreasing aggregation, for decreasing the inflammation and/or irritation accompanying the delivery of the pharmaceutical agent across membranes, and for facilitating receptor crosslinking.

An example of an additional entity which serves to provide a means for detection is a radiolabeling site, including radiolabeled chelates for cancer imaging or radiotherapy and for assessing dose regiments in different tissues. Examples of complexes utilizing lipids containing sites for radiolabeling are described herein, and in copending U.S. Provisional Patent Application Serial No. 60/308,347.

Other entities are capable of extending the excretion half-life of a pharmaceutical agent. Typically, these entities will find use with peptide and protein drugs or other pharmaceutical agents with short excretion half-lives. Generally, this modifier will comprise a moiety capable of binding to a serum protein, such as human serum albumin. Typically those moieties will be bound to plasma more than 60%, preferably more than 70%, more preferably more than 80%, and most preferably more than 90%, as measured by the procedures known in the art. Examples of such effector groups include naproxen, fluoxetine, oxazepam, nitrazepam, phenylbutazone, nortriptyline, methadone hydrochloride, lorazepam, imipramine, haloperidol, flurazepam, doxycycline, ditonin, diflunisal, diazoxide, diazepam, nordazepam, desipramine, dapsone, clofibrate, amantadine, chlorthalidone, clonazepam, chlorpropamide, chlorpromazine, chlorpheiramine, chloroquine, carbamazepine, auranofin, amitriptyline, amphotericin B, piroxicam, warfarin, pimozide, doxorubicin, pyrimethamine, amidoarone, protriptylene, desipramine, nortriptyline, oxazepam, nitrazepam, and tetrahydrocannabinols.

A receptor crosslinking functionality modifier is essentially a targeting modifier. Crosslinking of cell surface receptors is a useful ability for a pharmaceutical agent in that crosslinking is often a required step before receptor internalization. Thus, the crosslinking modifier can be used as a means to incorporate a pharmaceutical agent into a cell. In addition, the presence of two receptor binding sites (i.e., targeting modifiers) gives the pharmaceutical agent increased avidity.

A similar effect can also be obtained with an avidity modifier. In this case, each pharmaceutical agent will have a targeting modifier and an avidity modifier (i.e., a dimerization peptide). The dimerization of two peptides will effectively form one molecule with two targeting modifiers, thus allowing receptor crosslinking. With this bimolecular approach to crosslinking, the concentration dependence will be greater and increased targeting and crosslinking specificity can be obtained for tissues with high receptor density.

Alternatively, a functionality modifier may serve to prevent aggregation. Specifically, many peptide and protein pharmaceutical agents form dimers or larger aggregates which may limit their permeability or otherwise affect properties related to dosage form or bioavailability. For example, the hexameric form of insulin can be inhibited through the use of an appropriate functionality modifier and thus, result in greater diffusability of the monomeric form of insulin.

Large numbers of therapeutic entities may be attached to one linking carrier that may also bear from several to about one thousand targeting entities for in vivo adherence to targeted surfaces. The improved binding conveyed by multiple targeting entities can also be utilized therapeutically to block cell adhesion to endothelial receptors in vivo, for example. Blocking these receptors can be useful to control pathological processes, such as inflammation and control of metastatic cancer. For example, multi-valent sialyl Lewis X derivatized liposomes can be used to block neutrophil binding, and antibodies against VCAM-1 on polymerized liposomes can be used to block lymphocyte binding, e.g. T-cells.

Generally, lipids suitable for use in polymerized liposomes have an active head group for attaching one or more therapeutic entities or targeting entities, a spacer portion for accessibility of the active head group; a hydrophobic tail for self-assembly into liposomes; and a polymerizable group to stabilize the liposomes.

Targeted polymerized liposomes which recirculate in the vasculature may include endothelial antigens which interact with the cell adhesion molecules or other cell surface receptors to retain a number of the targeted polymerized liposomes at the desired location. The high concentration of therapeutic entities in the polymerized liposomes render possible site-specific delivery of high concentrations of drugs or other therapeutic entities, while minimizing the burden on other tissues. The polymerized liposomes described herein are particularly well-suited since they maintain their integrity in vivo, recirculate in the blood pool, are rigid and do not easily fuse with cell membranes, and serve as a scaffold for attachment of both the antibodies/targeting entities and the therapeutic entities. The size distribution, particle rigidity and surface characteristics of the polymerized liposomes can be tailored to avoid rapid clearance by the reticuloendothelial system and the surface can be modified with ethylene glycol to further increase intravascular recirculation times. In one embodiment, the polymerized liposomes were found to have blood pool half-lives of about 20 hours in rats.

In one embodiment, the site-specific polymerized liposomes having attached monoclonal antibodies for specific receptor targeting may be used to deliver therapeutic entities to cells expressing intercellular adhesion molecule-1, ICAM-1. This marker is upregulated in murine experimental autoimmune encephalitis, an animal model for multiple sclerosis.

Therapeutic Compositions

The present invention is also directed toward therapeutic compositions comprising the therapeutic agents of the present invention. Compositions of the present invention can also include other components such as a pharmaceutically acceptable excipient, an adjuvant, and/or a carrier. For example, compositions of the present invention can be formulated in an excipient that the animal to be treated can tolerate. Examples of such excipients include water, saline, Ringer's solution, dextrose solution, mannitol, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer, Tris buffer, histidine, citrate, and glycine, or mixtures thereof, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, the composition can also include an immunopotentiator, such as an adjuvant or a carrier. Adjuvants are typically substances that generally enhance the immune response of an animal to a specific antigen. Suitable adjuvants include, but are not limited to, Freund's adjuvant; other bacterial cell wall components; aluminum-based salts; calcium-based salts; silica; polynucleotides; toxoids; serum proteins; viral coat proteins; other bacterial-derived preparations; gamma interferon; block copolymer adjuvants, such as Hunter's Titermax adjuvant (Vaxcel.TM., Inc. Norcross, Ga.); Ribi adjuvants (available from Ribi ImmunoChem Research, Inc., Hamilton, Mont.); and saponins and their derivatives, such as Quil A (available from Superfos Biosector A/S, Denmark). Carriers are typically compounds that increase the half-life of a therapeutic composition in the treated animal. Suitable carriers include, but are not limited to, polymeric controlled release formulations, biodegradable implants, liposomes, bacteria, viruses, oils, esters, and glycols.

One embodiment of the present invention is a controlled release formulation that is capable of slowly releasing a composition of the present invention into an animal. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

Generally, the therapeutic agents used in the invention are administered to an animal in an effective amount. Generally, an effective amount is an amount effective to either (1) reduce the symptoms of the disease sought to be treated or (2) induce a pharmacological change relevant to treating the disease sought to be treated. For cancer, an effective amount includes an amount effective to: reduce the size of a tumor; slow the growth of a tumor; prevent or inhibit metastases; or increase the life expectancy of the affected animal.

Therapeutically effective amounts of the therapeutic agents can be any amount or doses sufficient to bring about the desired effect and depend, in part, on the condition, type and location of the cancer, the size and condition of the patient, as well as other factors readily known to those skilled in the art. The dosages can be given as a single dose, or as several doses, for example, divided over the course of several weeks.

The present invention is also directed toward methods of treatment utilizing the therapeutic compositions of the present invention. The method comprises administering the therapeutic agent to a subject in need of such administration.

The therapeutic agents of the instant invention can be administered by any suitable means, including, for example, parenteral, topical, oral or local administration, such as intradermally, by injection, or by aerosol. In the preferred embodiment of the invention, the agent is administered by injection. Such injection can be locally administered to any affected area. A therapeutic composition can be administered in a variety of unit dosage forms depending upon the method of administration. For example, unit dosage forms suitable for oral administration of an animal include powder, tablets, pills and capsules. Preferred delivery methods for a therapeutic composition of the present invention include intravenous administration and local administration by, for example, injection or topical administration. For particular modes of delivery, a therapeutic composition of the present invention can be formulated in an excipient of the present invention. A therapeutic reagent of the present invention can be administered to any animal, preferably to mammals, and more preferably to humans.

The particular mode of administration will depend on the condition to be treated. It is contemplated that administration of the agents of the present invention may be via any bodily fluid, or any target or any tissue accessible through a body fluid.

Preferred routes of administration of the cell-surface targeted therapeutic agents of the present invention are by intravenous, interperitoneal, or subcutaneous injection including administration to veins or the lymphatic system. While the primary focus of the invention is on vascular-targeted agents, in principle, a targeted agent can be designed to focus on markers present in other fluids, body tissues, and body cavities, e.g. synovial fluid, ocular fluid, or spinal fluid. Thus, for example, an agent can be administered to spinal fluid, where an antibody targets a site of pathology accessible from the spinal fluid. Intrathecal delivery, that is, administration into the cerebrospinal fluid bathing the spinal cord and brain, may be appropriate for example, in the case of a target residing in the choroid plexus endothelium of the cerebral spinal fluid (CSF)-blood barrier.

As an example of one treatment route of administration through a bodily fluid is one in which the disease to be treated is rheumatoid arthritis. In this embodiment of the invention, the invention provides therapeutic agents to treat inflamed synovia of people afflicted with rheumatoid arthritis. This type of therapeutic agent is a radiation synovectomy agent. Individuals with rheumatoid arthritis experience destruction of the diarthroidal or synovial joints, which causes substantial pain and physical disability. The disease will involve the hands (metacarpophalangeal joints), elbows, wrists, ankles and shoulders for most of these patients, and over half will have affected knee joints. Untreated, the joint linings become increasingly inflamed resulting in pain, loss of motion and destruction of articular cartilage. Chemicals, surgery, and radiation have been used to attack and destroy or remove the inflamed synovium, all with drawbacks.

The concentration of the radiation synovectomy agent varies with the particular use, but a sufficient amount is present to provide satisfactory radiation synovectomy. For example, in radiation synovectomy of the hip, the concentration of the agent will generally be higher than when used for the radiation synovectomy of the wrist joints. The radiation synovectomy composition is administered so that preferably it remains substantially in the joint for 20 half-lifes of the isotope although shorter residence times are acceptable as long as the leakage of the radionuclide is small and the leaked radionuclide is rapidly cleared from the body.

The radiation synovectomy compositions may be used in the usual way for such procedures. For example, in the case of the treatment of a knee-joint, a sufficient amount of the radiation synovectomy composition to provide adequate radiation synovectomy is injected into the knee-joint. There are a number of different techniques which can be used and the appropriate technique varies on the joint being treated. An example for the knee joint can be found, for example, in Nuclear Medicine Therapy, J. C. Harbert, J. S. Robertson and K. D. Reid, 1987, Thieme Medical Publishers, pages 172-3.

The route of administration through the synovia may also be useful in the treatment of osteoarthritis. Osteoarthritis is a disease where cartilage degradation leads to severe pain and inability to use the affected joint. Although age is the single most powerful risk factor, major trauma and repetitive joint use are additional risk factors. Major features of the disease include thinning of the joint, softening of the cartilage, cartilage ulcers, and abraded bone. Delivery of agents by injection of targeted carriers to synovial fluid to reduce inflammation, inhibit degradative enzymes, and decrease pain are envisioned in this embodiment of the invention.

Another route of administration is through ocular fluid. In the eye, the retina is a thin layer of light-sensitive tissue that lines the inside wall of the back of the eye. When light enters the eye, it is focused by the cornea and the lens onto the retina. The retina then transforms the light images into electrical impulses that are sent to the brain through the optic nerve.

The macula is a very small area of the retina responsible for central vision and color vision. The macula allows us to read, drive, and perform detailed work. Surrounding the macula is the peripheral retina which is responsible for side vision and night vision. Macular degeneration is damage or breakdown of the macula, underlying tissue, or adjacent tissue. Macular degeneration is the leading cause of decreased visual acuity and impairment of reading and fine “close-up” vision. Age-related macular degeneration (ARMD) is the most common cause of legal blindness in the elderly.

The most common form of macular degeneration is called “dry” or involutional macular degeneration and results from the thinning of vascular and other structural or nutritional tissues underlying the retina in the macular region. A more severe form is termed “wet” or exudative macular degeneration. In this form, blood vessels in the choroidal layer (a layer underneath the retina and providing nourishment to the retina) break through a thin protective layer between the two tissues. These blood vessels may grow abnormally directly beneath the retina in a rapid uncontrolled fashion, resulting in oozing, bleeding, or eventually scar tissue formation in the macula which leads to severe loss of central vision. This process is termed choroidal neovascularization (CNV).

CNV is a condition that has a poor prognosis; effective treatment using thermal laser photocoagulation relies upon lesion detection and resultant mapping of the borders. Angiography is used to detect leakage from the offending vessels but often CNV is larger than indicated by conventional angiograms since the vessels are large, have an ill-defined bed, protrude below into the retina and can associate with pigmented epithelium.

Neovascularization results in visual loss in other eye diseases including neovascular glaucoma, ocular histoplasmosis syndrome, myopia, diabetes, pterygium, and infectious and inflammatory diseases. In histoplasmosis syndrome, a series of events occur in the choroidal layer of the inside lining of the back of the eye resulting in localized inflammation of the choroid and consequent scarring with loss of function of the involved retina and production of a blind spot (scotoma). In some cases, the choroid layer is provoked to produce new blood vessels that are much more fragile than normal blood vessels. They have a tendency to bleed with additional scarring, and loss of function of the overlying retina. Diabetic retinopathy involves retinal rather than choroidal blood vessels resulting in hemorrhages, vascular irregularities, and whitish exudates. Retinal neovascularization may occur in the most severe forms. When the vasculature of the eye is targeted, it should be appreciated that targets may be present on either side of the vasculature.

Delivery of the agents of the present invention to the tissues of the eye can be in many forms, including intravenous, ophthalmic, and topical. For ophthalmic topical administration, the agents of the present invention can be prepared in the form of aqueous eye drops such as aqueous suspended eye drops, viscous eye drops, gel, aqueous solution, emulsion, ointment, and the like. Additives suitable for the preparation of such formulations are known to those skilled in the art. In the case of a sustained-release delivery system for the eye, the sustained-release delivery system may be placed under the eyelid or injected into the conjunctiva, sclera, retina, optic nerve sheath, or in an intraocular or intraorbitol location. Intravitreal delivery of agents to the eye is also contemplated. Such intravitreal delivery methods are known to those of skill in the art. The delivery may include delivery via a device, such as that described in U.S. Pat. No. 6,251,090 to Avery.

In a further embodiment, the therapeutic agents of the present invention are useful for gene therapy or gene delivery. As used herein, the phrases “gene therapy” or “gene delivery” refer to the transfer of genetic material (e.g., DNA or RNA) of interest into a host to treat or prevent a genetic or acquired disease or condition. The genetic material of interest encodes a product (e.g., a protein polypeptide, peptide or functional RNA) whose production in vivo is desired. For example, the genetic material of interest can encode a hormone, receptor, enzyme or polypeptide of therapeutic value. In a specific embodiment, the subject invention utilizes a class of lipid molecules for use in non-viral gene therapy which can complex with nucleic acids as described in Hughes, et al., U.S. Pat. No. 6,169,078, incorporated by reference herein in its entirety, in which a disulfide linker is provided between a polar head group and a lipophilic tail group of a lipid.

These therapeutic compounds of the present invention effectively complex with DNA and facilitate the transfer of DNA through a cell membrane into the intracellular space of a cell to be transformed with heterologous DNA. Furthermore, these lipid molecules facilitate the release of heterologous DNA in the cell cytoplasm thereby increasing gene transfection during gene therapy in a human or animal.

Cationic lipid-polyanionic macromolecule aggregates may be formed by a variety of methods known in the art. Representative methods are disclosed by Felgner et al., supra; Eppstein et al. supra; Behr et al. supra; Bangham, A. et al. M. Mol. Biol. 23:238, 1965; Olson, F. et al. Biochim. Biophys. Acta 557:9, 1979; Szoka, F. et: al. Proc. Natl. Acad. Sci. 75: 4194, 1978; Mayhew, E. et al. Biochim. Biophys. Acta 775:169, 1984; Kim, S. et al. Biochim. Biophys. Acta 728:339, 1983; and Fukunaga, M. et al. Endocrinol. 115:757, 1984. In general aggregates may be formed by preparing lipid particles consisting of either (1) a cationic lipid or (2) a cationic lipid mixed with a colipid, followed by adding a polyanionic macromolecule to the lipid particles at about room temperature (about 18 to 26° C.). In general, conditions are chosen that are not conducive to deprotection of protected groups. In one embodiment, the mixture is then allowed to form an aggregate over a period of about 10 minutes to about 20 hours, with about 15 to 60 minutes most conveniently used. Other time periods may be appropriate for specific lipid types. The complexes may be formed over a longer period, but additional enhancement of transfection efficiency will not usually be gained by a longer period of complexing.

The compounds and methods of the subject invention can be used to intracellularly deliver a desired molecule, such as, for example, a polynucleotide, to a target cell. The desired polynucleotide can be composed of DNA or RNA or analogs thereof. The desired polynucleotides delivered using the present invention can be composed of nucleotide sequences that provide different functions or activities, such as nucleotides that have a regulatory function, e.g., promoter sequences, or that encode a polypeptide. The desired polynucleotide can also provide nucleotide sequences that are antisense to other nucleotide sequences in the cell. For example, the desired polynucleotide when transcribed in the cell can provide a polynucleotide that has a sequence that is antisense to other nucleotide sequences in the cell. The antisense sequences can hybridize to the sense strand sequences in the cell. Polynucleotides that provide antisense sequences can be readily prepared by the ordinarily skilled artisan. The desired polynucleotide delivered into the cell can also comprise a nucleotide sequence that is capable of forming a triplex complex with double-stranded DNA in the cell. The desired polynucleotide delivered into the cell can interfere with biological pathways of the cell, thereby resulting in cell death.

Imaging

The present invention is directed to imaging agents displaying important properties in medical diagnosis. More particularly, the present invention is directed to magnetic resonance imaging contrast agents, such as gadolinium, ultrasound imaging agents, or nuclear imaging agents, such as Tc-99m, In-111, Ga-67, Rh-105, I-123, Nd -147, Pm-151, Sm-153, Gd-159, Tb-161, Er-171, Re-186, Re-188, and Tl-201.

This invention also provides a method of diagnosing abnormal pathology in vivo comprising, introducing a plurality of targeting image enhancing polymerized particles targeted to a molecule involved in the abnormal pathology into a bodily fluid contacting the abnormal pathology, the targeting image enhancing polymerized particles attaching to a molecule involved in the abnormal pathology, and imaging in vivo the targeting image enhancing polymerized particles attached to molecules involved in the abnormal pathology. Such methods are described in the EXAMPLES section, and also in copending U.S. Provisional Patent Application No. 60/308,347.

Exemplary Lipid Constructs and Uses

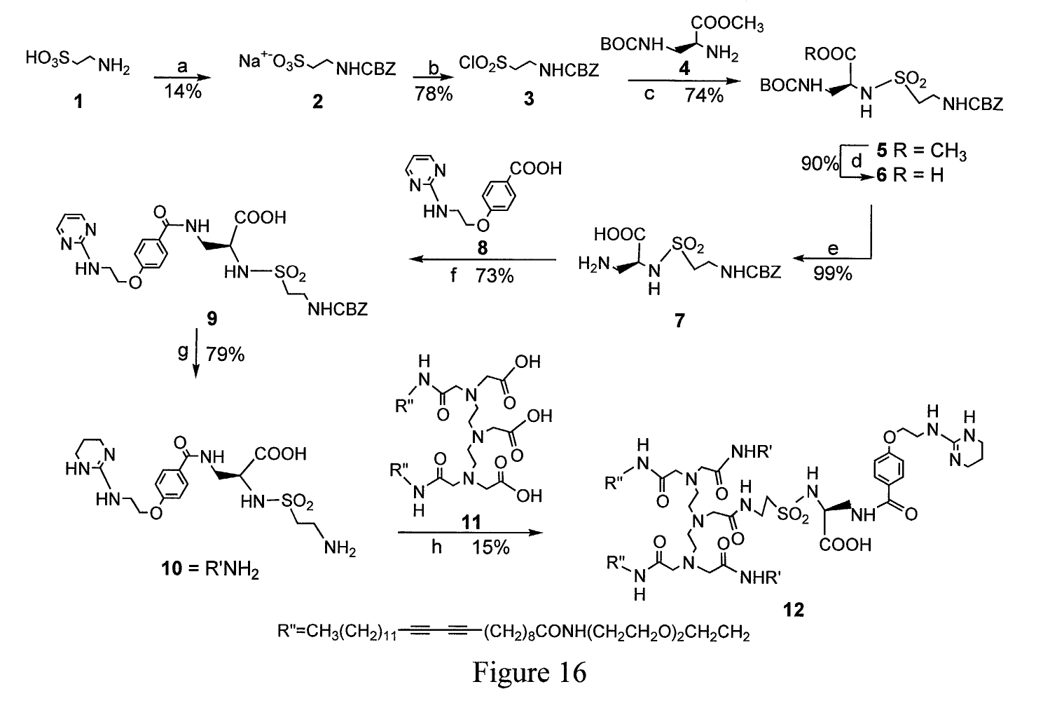

Integrin-targeted PVs consist of a phosphocholine (PC) lipid for biocompatibility, a lipid derivative of diethylenetriamine pentaacetic acid (DTPA) to impart colloidal stability and allow for in vitro binding assays, and a targeting lipid with a head group derived from the αᵥβ₃ integrin binding ligand 3-{4-[2-(3,4,5,6-tetrahydropyrimidin-2-ylamino)-ethyloxy]-benzoylamino}-2(S)-benzene-sulfonyl-aminopropionic acid, compound 10 in FIG. 16. FIG. 16 outlines the preparation of novel trivalent lipid-integrin antagnoist 12, used in the preparation targeting agents of the present invention.

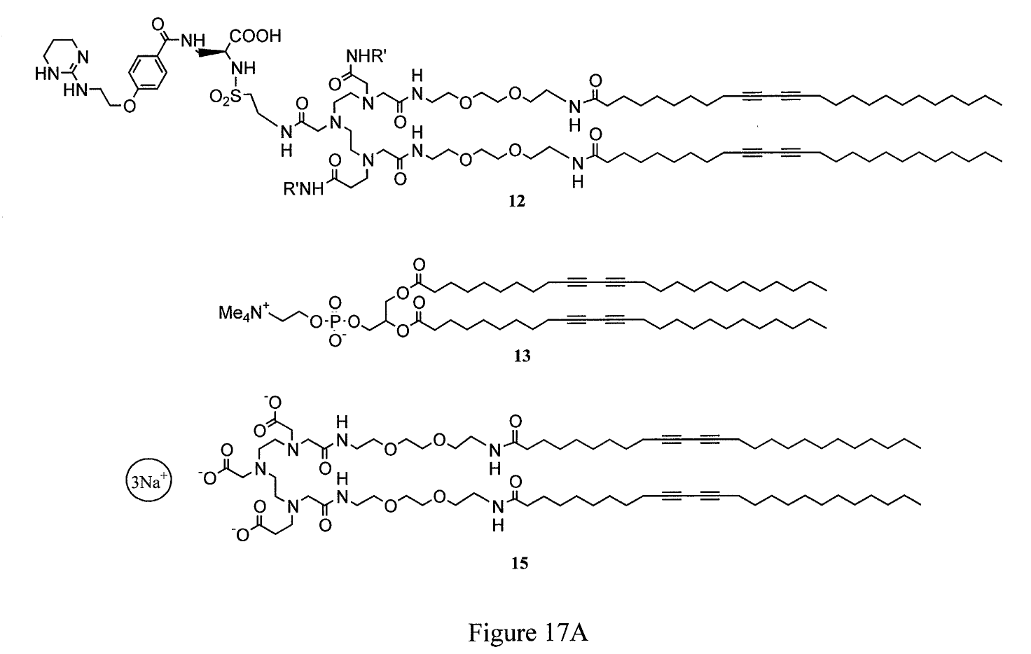

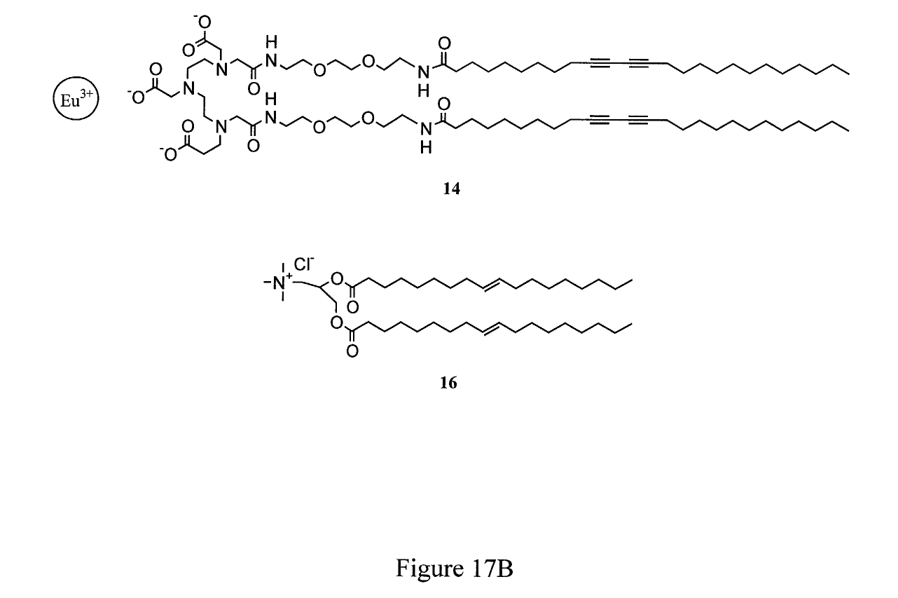

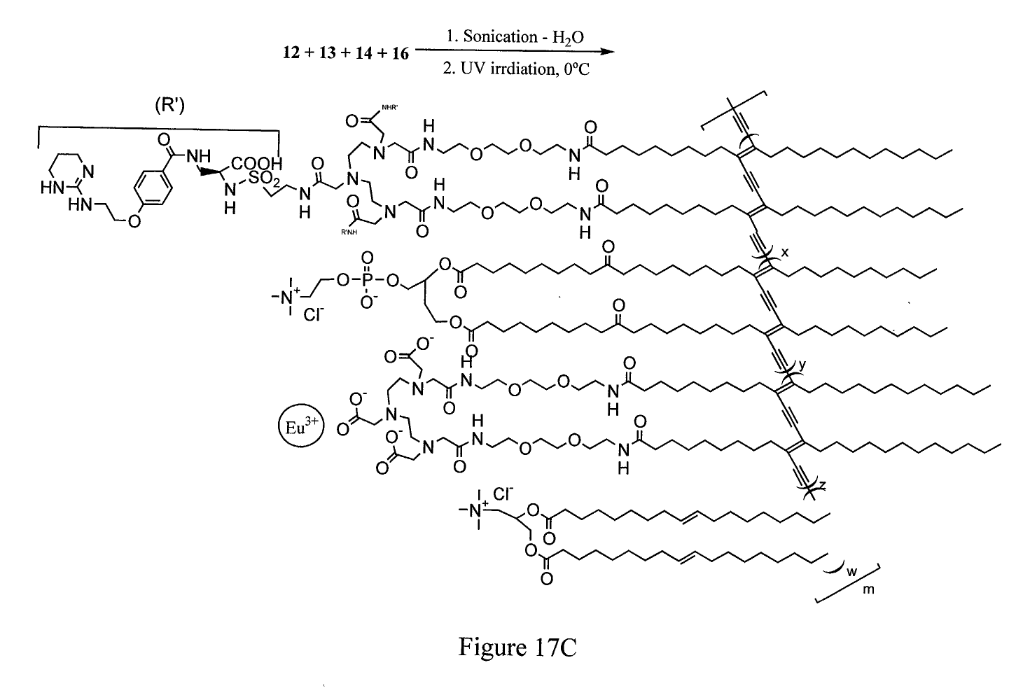

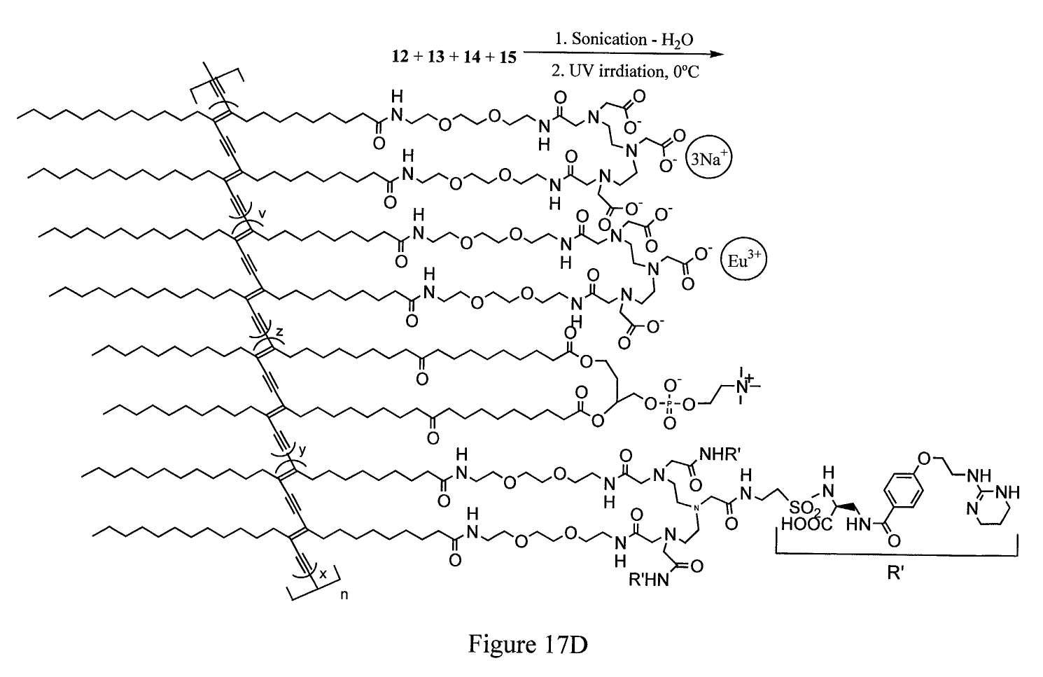

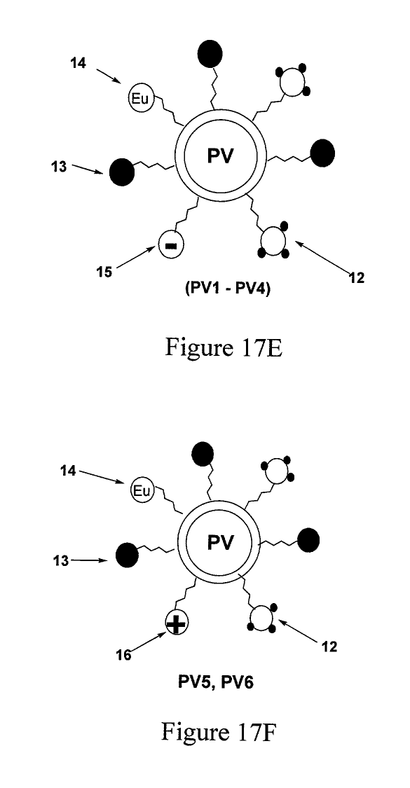

FIG. 17 outlines the formation of the nanoparticles (PVs) by self-assembly and polymerization of the appropriate lipids as previously described in Storrs, et al., ibid. The trivalent lipid-integrin antagnoist 12 was combined with commercially available diacetylene phospholipid 13 and the europium-chelator lipid complex 14 in a chloroform solution. Compound 14 was added at one percent to all formulations in order to visualize the particles using Fluorescence spectroscopy. Orellana, et al., Biochim. Biophys. Acta (1996) 1284:29-34. To this solution was added either the anionic chelator lipid 15 or the cationic lipid 16 in order to vary the surface charge and provide a surface to chelate radionuclides. Storrs, et al., 1995b. The surface density of the integrin antagonist on the PVs was controlled by varying the concentration of compound 12. To form vesicles, the combined lipid solutions were evaporated to dryness and dried under high vacuum to remove any residual solvent. The dried lipid film was hydrated to a knows lipid density (30 mM) using deionized water. The resulting suspension was then sonicated at temperatures above the gel-liquid crystal phase transition (Tₘ≈64° C.) using a probe-tip sonicator while maintaining the pH between 7.0 and 7.5. Spevak, W. R. Doctoral Thesis, University of California at Berkeley, 1993; Leaver, et al., Biochim. Biophys. Acta (1983) 732:210-218. After approximately one hour of sonication the solution became clear. The vesicles were then polymerized by cooling the solution to 0° C. on a bed of wet ice and irradiating the solution at 254 nm with a hand-held UV lamp for two hours. The resulting PVs (PV1 through PV6) were yellow-orange in color and had two visible absorption bands centered at 490 nm and 535 nm arising from the conjugated ene-yne diacetylene polymer. Storrs, et al., 1995a. The mean diameter of the PVs were between 40 nm and 50 nm as determined by dynamic light scattering (DLS) and the zeta potential was between −42 and −53 mV for PV1 through PV4 and +35 and +43 mV for PV5 and PV6 respectively (Brookhaven Instruments, Holtsville, N.Y.). The PVs were stable for months without significant changes in the physical and biological properties when formulated for in vivo applications using 150 mM sodium chloride, 50 mM histidine, and 5% dextrose solutions. Properties of exemplary PVs are shown in Table Z.

TABLE Z
Composition and physical properties of the PVs

| | mol % | | | | | | Zeta Potential |
|---|---|---|---|---|---|---|---|
| | 12 | 13 | 14 | 15 | 16 | Size (nm) | (mV) |
| PV1 | 10.0 | 79.0 | 1.0 | 10.0 | 0.0 | 45.1 ± 0.6 | −42 ± 1.3 |
| | | | | | | | (anionic) |
| PV2 | 1.0 | 88.0 | 1.0 | 10.0 | 0.0 | 42.8 ± 1.5 | −49 ± 0.8 |
| | | | | | | | (anionic) |
| PV3 | 0.1 | 88.9 | 1.0 | 10.0 | 0.0 | 44.4 ± 0.8 | −53 ± 1.1 |
| | | | | | | | (anionic) |
| PV4 | 0.0 | 89.0 | 1.0 | 10.0 | 0.0 | 46.4 ± 0.7 | −49 ± 0.3 |
| | | | | | | | (anionic) |
| PV5 | 10.0 | 59.0 | 1.0 | 0.0 | 30.0 | 41.7 ± 2.2 | 35 ± 1.1 |
| | | | | | | | (cationic) |
| PV6 | 0 | 69 | 1 | 0 | 30 | 36.8 ± 0.9 | 43 ± 0.6 |
| | | | | | | | (cationic) |

PV's were also prepared containing αᵥβ₃ integrin agonist-lipid compound 12 at 1-30 mole percent along with 1,2-bis(10,12-tricosadiynoyl)-sn-glycero-3-phosphocholine (BisT-PC, 13) at 99-70 mole percent. Liposomes and PV's containing agonist-lipid compound 12 are referred to herein as “integrin targeted liposomes” or ITLs.

To evaluate binding of PVs in vitro, vesicles were labeled with europium and binding was monitored in 96 well plates coated with the avP₃ integrin by time resolved fluorescence (TRF), as described in EXAMPLE 5. TRF signal was 6 fold higher for PVs than signal for non-targeting liposomes. Specific targeting was also demonstrated in a competition assay where signal from ITL-Eu complexes was reduced by the integrin ligand without the lipid side chain.

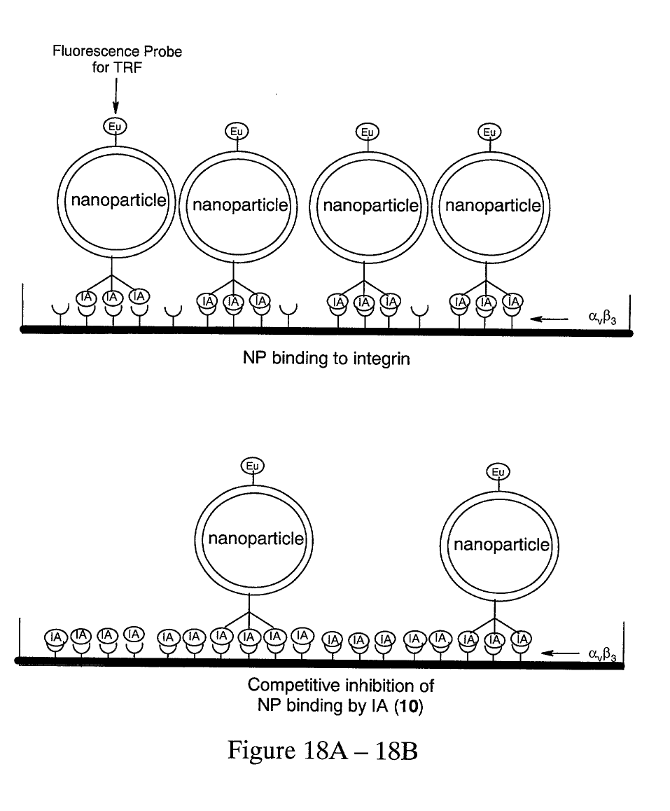

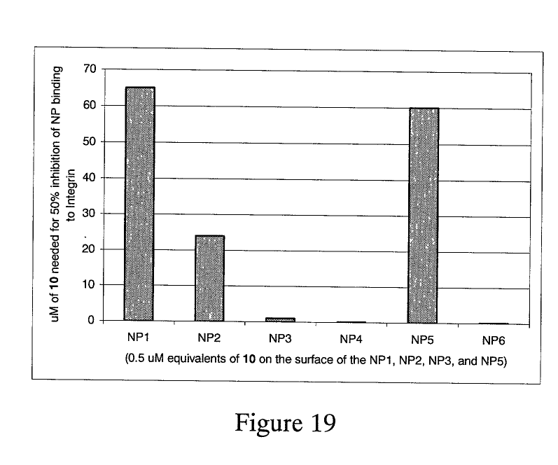

In order to assess the utility of the PVs in targeting the integrins as desribed in EXAMPLE 5, polymerized vesicles were constructed using 0.1, 1 and 10 mol % of integrin antagonist lipid complex compound 12 and compounds 13-16 as outlined in Table Z. The materials that contained 10 mol % of compound 12 (PV1 and PV5) had the highest avidity for the integrin αᵥβ₃. In a competitive integrin binding assay the PVs (PV1-PV5) were mixed with various concentration of 10 and then added to a 96 well plate previously coated with αᵥβ₃ integrin. The unbound PVs and integrin antagonist 10 were washed away and the bound PVs were measured using the Europium present in the PVs (Wallac, Gaitherburg, Md.) (FIGS. 18A-18B). It took over 100 fold of the free ligand 10 (65 μM) to achieve 50% inhibition of PV1 that had only the equivalent of 0.5 μM of the integrin antagonist 10 on its surface as shown in FIG. 19.

In an in vitro assay for inhibition of cell adhesion using αᵥβ₃ positive M21, described in EXAMPLE 6, Melanoma cells binding to vitronectin coated plates, the IC₅₀ for the free ligand 10 was 64 μM. In sharp contrast, the IC₅₀ for the anionic particle PV1 was 0.27 μM equivalents of compound 10 on the surface. This results in over 200 times greater avidity to the cell surface when 10 is on the NPs compared to the free ligand. Also for the cationic particle PV5, the IC₅₀ was 0.35 μM equivalents of compound 10 which is approximately 180 times greater avidity when compared with free ligand (Table 3).

TABLE 3
Cell adhesion inhibition assay

| | mol % of | Cell Adhesion Assay IC₅₀ | IC₅₀ for 10 |
|---|---|---|---|
| Material | lipid 12 | (μM of 10 on NPs) | +E,OVR IC₅₀ for NP |
| PV1 | 10 | 0.27 | 237 |
| PV2 | 1 | 7 | 9 |
| PV3 | 0.1 | 30.5 | 2 |
| PV4 | 0 | No Inhibition | x |
| PV5 | 10 | 0.35 | 183 |
| PV6 | 0 | No Inhibition | x |
| Compound 10 | x | 63.9 | x |

Thus regardless of the surface charge, the PVs had approximately 200 times increased avidity to the integrins when compared to the monomeric ligand. This demonstrates that a robust interaction occurs between the PV surface and the surface of the cell. This interaction is independent of surface charge on the PVs and is directly related to a specific receptor ligand interaction. Thus an increase of approximately two orders in magnitude of avidity can be achieved by multivalent presentation of an integrin antagonist on the surface of the PVs compared to the free ligand. When the amount of compound 12 in the PV formulations was decreased by 10 fold and 100 fold to 1 mol % and 0.1 mol % to give PV2 and PV3 respectively, the capacity to block cell adhesion decreased by approximately one and two orders of magnitude (Table 3).

The cell adhesion assay was also performed with plates coated with collagen. Collagen binds to collagen receptors (α₂β₁ integrins) but not αᵥβ₃ integrins. In this case, it was observed that the PV-integrin agonist inhibited cell adhesion, whereas neither the PV alone nor the agonist alone inhibited cell adhesion. Since neither component alone showed inhibition, it is clear that the individual components don't bind to collagen receptors. Without being bound by theory, it is believed that the observed inhibition of cell adhesion by PV-integrin agonist is due to the PV preventing interaction of collagen and its receptor by steric hindrance, due to the large size of the PV. Thus, not only does the PV targeted to a specific receptor bind to the receptor on the cell surface, but it blocks access to adjacent receptors due to its steric bulk.

To evaluate binding of ITLs in vitro, vesicles were labeled with europium and binding was monitored in 96 well plates coated with the αᵥβ₃ integrin by time resolved fluorescence (TRF). TRF signal was 6 fold higher for ITLs than signal for non-targeting liposomes. Specific targeting was also demonstrated in a competition assay where signal from ITL-Eu complexes was reduced by the integrin ligand without the lipid side chain.

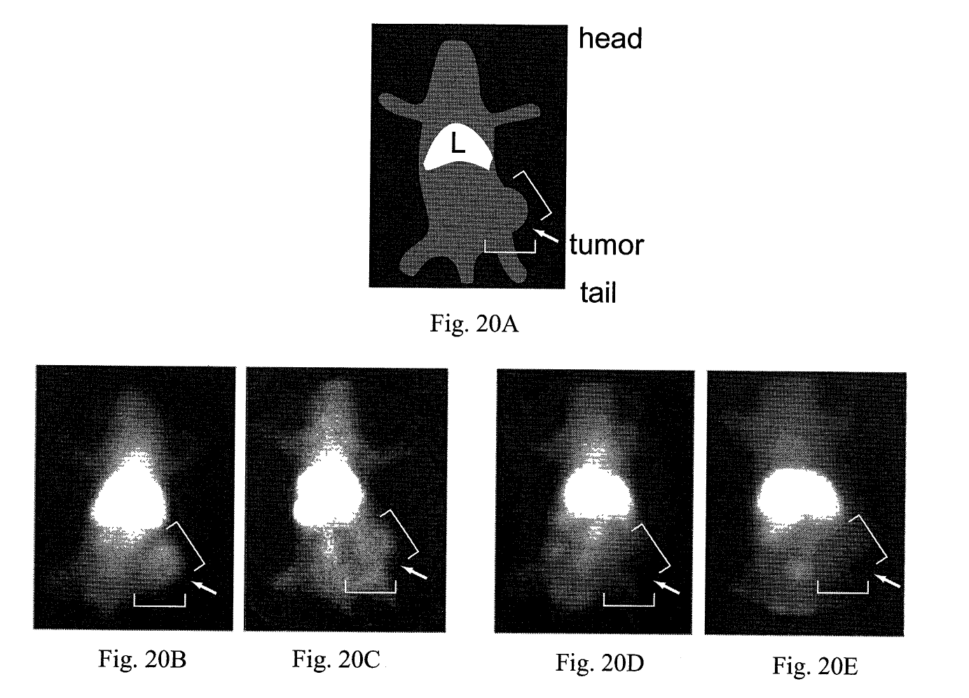

Paramagnetic PVs are useful for imaging tumors in vivo, as described in EXAMPLE 7. These materials can, therefore, serve as spatial and temporal imaging agents that have high avidity for the integrins in vivo. In additin to the 200-fold increase in avidity of the PVs in the cell adhesion assay compared to the free ligand, this effect is also observed in vivo by showing that a significant uptake of the PVs containing the integrin antagonist on the surface occurs in a melanoma tumor model and persists at the tumor site even after 24 hours, as shown in FIG. 20A-E.

Quantitative encapsulation of doxorubicin at 0.15 and 1.5 mg/mL was achieved in 10% sucrose solution using vesicles (15 mg/mL) containing 250 mM ammonium sulfate as described in EXAMPLE 11-EXAMPLE 14. Targeted delivery of doxorubicin by ITLs was demonstrated with murine endothelial cells (MECs) in an in vitro cell proliferation assay described in, but the murine tumor cells were resistant to treatment under identical assay conditions. For MECs, incubation with ITLdox resulted in 4-fold higher reduction in cell density than untargeted Ldox. ITLs without doxorubicin had no effect on cell proliferation. Doxorubicin at identical concentrations also resulted in significant reductions in cell proliferation, but analysis of the vesicles by size exclusion chromatography shows that reductions in cell proliferation were not due to release of doxorubicin from ITLdox.

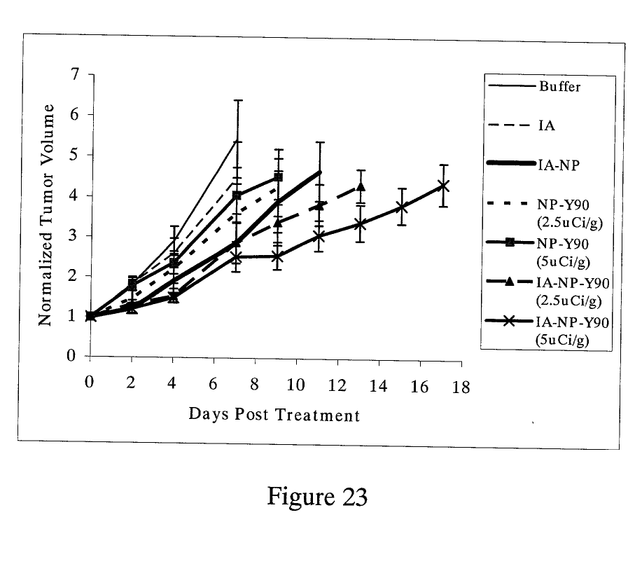

Statistically significant differences in tumor growth rate were observed for ITLdox in the syngeneic K1735-M2 murine melanoma model (ANOVA P<0.001). One to one comparisons indicate significantly reduced tumor growth associated with ITLdox treatment at doxorubicin doses of 1 and 10 μg/g relative to control treatments including 10% sucrose, Ldox, and ITL (Tukey's W procedure P=0.048 to P<0.001). Lipid doses were 100 μg/g. The data are shown in FIG. 23.

The EXAMPLES section also describes a number of other procedures, including encapsulation of other therapeutic entities, association of other targeting entities, entities with differing lipid compositions, association of therapeutic radioisotopes and the like.

Although only a few embodiments of the present invention have been described, it should be understood that the present invention may be embodied in many other specific forms without departing from the spirit or the scope of the present invention.

### EXAMPLES

### Example 1

### General Methods

All solvents and reagents used were of reagent grade. Solvent evaporations were performed under reduced pressure provided from house vacuum or a Welch direct drive vacuum pump at ≦40° C. ¹H and ¹³C-NMR spectra were recorded on a JEOL FX90Q at 90 MHz in CDCl₃, CD₃OD, D₂O or blends thereof as described for each case. (Note: although soluble in CDCl₃, the addition of CD₃OD to the lipids inhibits formation of inverted micelles and thus provided sharper spectra. Spectra were referenced to residual CHCl₃ (7.25 ppm) for ¹H experiments and the center line of CDCl₃ (77.00 ppm) for ¹³C experiments. MALDI-TOF mass spectrometry was performed on PerSeptive DE instrument (Mass Spectrometry, The Scripps Resea ch Institute, La Jolla, Calif.). TLC was performed on glass backed Merck 60 F254 (0.2 mm; EM Separations, Wakefield, R.I.) and the developed plates routinely sprayed with ceric sulfate (1%) and ammonium molybdate (2.5%) in 10% aqueous sulfuric acid and heated to ≈150° C. Other developers include iodine (general use), 0.5% ninhydrin in acetone (for amines), and ultraviolet light (for chromophores).

### Example 2

### Preparation of 4-[2-(3,4,5,6-Tetrahydro-Pyrimidin-2-Ylamino)Ethyloxy]Benzoyl-2-(S)Amino Ethylsulfonylamino-β-Alanine

A. Preparation of N-Benzyloxycarbonyl-taurine sodium salt (2). Taurine, 1 (40g, 320 mmol) dissolved in 4N sodium hydroxide solution (80 mL) and water 1,200 mL). To this solution was added benzyloxycarbonyl chloride, (48 mL, 330 mmol) drop wise, with vigorous stirring during a period of 4 hours. The pH was maintained alkaline by the addition of 10% sodium bicarbonate solution (300 mL) and 4N sodium hydroxide solution (45 mL). The reaction mixture was then washed with ether (1000 mL) and the aqueous layer was spin evaporated to dryness, dried under high vacuum over phosphorous pentoxide overnight to yield 12.70 g (14.1%) of 2. ¹H-NMR (D₂O): δ 7.50 (5H, s, Ar—H), 5.21 (2H, s, Ar—CH₂), 3.62 (2H, t, CH₂), 3.14 (2H, t, CH₂).

B. Preparation of 2-Benzyloxycarbonylaminoethanesulfonyl chloride (3). N-CBZ Taurine sodium 2 (12.7 g, 32 mmol) was suspended in dry diethyl ether (30 mL) under a positive pressure of argon and treated with phosphorous pentachloride (7 g, 33.6 mmol) in 5 portions over 15 minutes. The reaction was stirred for 4 h, at ambient temperature. The solvent was removed by spin evaporation. Ice water (10 mL) was added and the residue was triturated after cooling the flask and the contents in an ice bath. More ice water (50 mL) was added and the product solidified. The solids were collected by filtration washed with ice water (20 mL) and dried over phosphorous pentoxide overnight to yield 6.95 g (78.0%) of 3. ¹H-NMR (CDCl₃): δ7.35 (5H, s, Ar—H), 5.12 (2H, s, Ar—CH₂), 3.89 (2H, t, CH₂) overlapping with 3.85 (2H, t, CH₂).

C. Prepration of Methyl 3-butyloxycarbonylamino-2-(S)benzyloxycarbonyl-aminoethylsulfonylaminopropionate (5). A mixture of the sulfonyl chloride 3 (21.6 g, 78.0 mmol) and methyl-3-N-butoxycarbonylamine-2-aminopropionate (4, 9.96g, 39.2 mmol) in anhydrous tetrahydrofuran (150 mL) under a positive pressure of argon was cooled in an ice bath. To this solution was added N-methylmorpholine (16 mL, 145 mmol) in anhydrous THF (275 mL) drop wise during a period of 30 min using a dropping funnel previously dried and under a positive pressure of argon. After 1 h stirring in the ice bath, by TLC it was observed that all the sulfonyl chloride (R_{f}=0.65) had disappeared (eluent: ethyl acetate/hexane 1:1); however, there was unreacted diaminopropionic acid (R_{f}=0.1, ninhydrin spray) still present. More sulfonyl chloride (5.0 g, 18 mmol) was added during a period of 3 h. The reaction was then filtered and spin evaporated to remove the solvent and dissolved in ethyl acetate (100 mL) and washed with cold dilute hydrochloric acid (20 mL), saturated sodium bicarbonate solution (20 mL) and saturated sodium chloride solution (20 mL) and dried over anhydrous sodium sulfate. The solvent removed by spin evaporation and dried under vacuum over night. The residue was recrystallized by first dissolving in ethyl acetate and then by adding equal volume of hexane to obtain 5 as a colorless solid 13.4 g (74.3%). ¹H-NMR (CDCl₃): 6 7.36 (5H, s, Ar—H), 5.83 (1H, d, NH), 5.55 (1H, t, NH), 5.12 (2H, s, Ar—CH₂), 5.06 (1H, t, NH), 4.26 (2H, m, CH), 3.79 (3H, s, CH₃), 3.70 (2H, dd, CH₂), 3.26 (2H, dd, CH₂), 1.43 (9H, s, (CH₃)₃).

D. Preparation of 3-butyloxycarbonylamino-2-(S)-benzyloxycarbonylaminoethyl-sulfonylaminopropionic acid (6). A solution of the methyl ester 5 (13.3 g, 28.9 mmol) in tetrahydrofuran (160 mL) was cooled in an ice bath and to this solution was added a solution of lithium hydroxide (5.42 g, 128 mmol) in ice water (160 mL). The reaction mixture was slowly warmed to ambient temperature by removing the ice bath and the mixture was stirred at ambient temperature for 1 h. The organic solvent was then removed by spin evaporation. The residual aqueous portion was washed with diethyl ether (20 mL) and then acidified to pH 4 using diluted hydrochloric acid. This solution was cooled in an ice bath and then mixed with ethyl acetate (100 mL) and then further acidified to pH 1 using ice-cold diluted hydrochloric acid and immediately extracted with ethyl acetate (2×200 mL). The ethyl acetate layer was washed with brine (50 mL) and dried over anhydrous sodium sulfate. The solvent was then removed by spin evaporation and dried under high vacuum overnight to obtain 13.3 g of a foamy solid, which was recrystallized from hexane/ethyl acetate (1:1) to obtain 11.6 g (89.7%) of 6. ¹H-NMR (CDCl₃): δ7.33 (5H, s, Ar—H), 6.12 (1H, d, NH), 5.68 (1H, t, NH), 5.26 (1H, t, NH), 5.1 (2H, s, Ar—CH₂), 4.24 (2H, m, CH₂), 3.67 (1H, t, CH₂), 3.27 (2H, t, CH₂), 1.45 (9H, s, C(CH₃)₃).

E. Preparation of 3-amino-2-(S)-benzyloxycarbonylaminoethylsulfonylamino-propionic acid (7). N-BOC-β-amino acid 6 (11.5 g, 25.8 mmol) was treated with trifluoroacetic acid (68 mL) in methylene chloride (350 mL) for 1.5 h and then spin evaporated to dryness. The residue was dissolved in water (200 mL) and lyophilized to obtain 7 as a solid of 10.9 g (98.8%) of the β-amino acid. ¹H-NMR (CDCl₃): δ 7.30 (5H, s, Ar—H), 6.07 (1H, d, NH), 5.61 (1H, t, NH), 5.20 (1H, t, NH), 5.17 (2H, s, Ar—CH₂), 4.11 (2H, m, CH₂), 3.53 (2H, t, CH₂), 3.32 (2H, t, CH₂). DCI-MS for C₁₃H₁₉N₃O₆S: m/z (ion) 346 (M+H) (calculated for C₁₃H₁₉N₃O₆S+H, m/z 346).

F. Preparation of 4-[2-(pyrimidin-2-ylamino)ethyloxy]benzovl-2-(S)-benzyloxycarbonylaminoethylsulfonylamino-β-alanine (9). The benzoic acid derivative 8 (6.4 g, 24.7 mmol) and N-hydroxysuccinimide (3.6 g, 31 mmol) were dissolved in anhydrous dimethylsulfoxide (110 mL), under a positive pressure of argon and cooled in an ice bath. To this solution was added 1-(3(dimethylamino)propyl)-3-ethylcarbodiimide hydrochloride (4.9 g, 25.6 mmol). The solution was stirred at ice-cold temperature for 1 h and then allowed to warm to ambient temperature and continued to stir at room temperature for another 24 h. To this mixture was added a solution of the β-amino acid 7 (12.2 g, 25.8 mmol) followed by N-methymorpholine and stirred under argon for 3 days. The mixture was then poured into water 1 L) and acidified with diluted hydrochloric acid to pH 1.5 and extracted with ethyl acetate (5×500 mL). The organic phase was washed with saturated sodium chloride solution (50 mL) and then dried over anhydrous sodium sulfate. The solvent was removed by spin evaporation and the residue was triturated in ethyl acetate, filtered and dried under high vacuum to obtain 10.5 g (72.5%) of 9. ¹H-NMR (DMSO-d₆): δ 8.30 (2H, d Ar—H), 7.99 (2H, d, Ar—H), 7.34 (5H, s, Ar—H), 7.00 (2H, d, Ar—H), 6.60 (1H, dd, Ar—H), 5.01 (2H, s, CH₂), 4.15 (1H, t, CH), 3.67 (2H, t, CH₂), 3.56 (2H, t, CH₂), 3.17 (2H, t, CH₂).

G. Preparation of 4-[2-(3,4,5,6-Tetrahydropyrimidin-2-ylamino)ethyloxy]-benzoyl-2-(S)amino ethylsulfonylamino-β-alanine (10=R′NH₂ A solution of the pyrimidine derivative 10 (3.7 g, 6.4 mmol) was dissolved in acetic acid (190 mL) and concentrated hydrochloric acid (17 mL). This solution was treated with 10% palladium over carbon (1.62 g) and hydrogenated at 45 psi of hydrogen gas for 5 h. The mixture was then filtered through celite and washed with water. The solvent was removed by spin evaporation and dried under high vacuum. The residue was dissolved in water≈100 mL) and pH adjusted to 7.0 with 1N sodium hydroxide solution and then spin evapo ated to dryness. The residue was dissolved in methanol (20 mL) and filtered. The filtrate was spin evaporated and dissolved in water (275 mL) and lyophilized. The lyophilized product was then recrystallized from water to obtain 2.96 g (78.9%) of pure product. ¹H-NMR (D₂O): δ 7.80 (2H, d, Ar—H), 7.14 (2H, d, Ar—H), 4.49 (1H, s, CHaHb), 4.28 (1H, t, CH₂), 3.94 (1H, dd, CHₐH_{b}), 3.61 (6H, m, CH₂), 3.32 (4H, t, CH₂), 1.90 (2H, t, CH₂). ES-MS for C₁₈H₂₈N₆O₆S: m/z (ion) 457 (M+H) (calculated for C₁₈H28N₆O₆S+H, m/z 457).

H. Determination of chiral purity of 4-[2-(3,4,5,6-Tetrahydropyrimidin-2-ylamino)ethyloxy]benzoyl-2-(S)-aminoethylsulfonylamino-β-alanine. To 1 mL of a solution of 10 (1.4 mg in 636 μL of water and 636 μL of acetone) was added 1 mL of a solution of Marfey's reagent (1.4 mg/mL). To the turbid solution was added 500 μL of acetone, 1.5 mL of water, and 400 μL of 1M NaHCO₃ solution and incubated at 40° C. for 24 h. The solution was then neutralized with 200 μL of 2M hydrochloric acid solution and analyzed by HPLC. A control solution made without 10 was also treated similarly and analyzed by HPLC. A sample of 10 was epimerized by heating it to melt. The epimerized compound was treated similar to 10. The 10 sample showed only the SS diastereomer and the SR diastereoisomer was completely absent indicating the %ee was >99% (tR=12.2 min for SS diastereoisomer and 10.8 min for SR diastereoisomer)

I. Synthesis of Compound 12 Compound 11 (69 mg, 50 μmole) was dissolved in anhydrous CH₃CN (5 mL), anhydrous CH₂Cl₂ (2 mL) and Et₃N (1 mL) in a 3-neck RB flask, previously flame dried and filled with argon. To this solution was added the BOP reagent (134 mg 150 lmole) and the reaction was stirred well for 5 minutes. A solution of 10 (69 mg, 150 μmoles) was prepared in a dry vial filled with argon, in a mixture of anhydrous CH₃CN (5 mL) and anhydrous DMF (2 mL). The cloudy solution of 10 was added to the lipid solution using a dry syringe with continuous stirring. The reaction was allowed to stir for 10 h in dark. TLC (solvent: CHCl₃, CH₃OH, H₂O, and CH₃COOH (73:27:4:1) showed complete disappearance of the starting material (R_{f}=0.53). There was one major product (R_{f}=0.2) and 5 minor products (R_{f}<0.16). The solvent was removed by evaporation and dried under high vacuum for 24 hours. The crude product was purified by normal phase HPLC using a semi preparative silica column, flow rate 5 mL/min, isocratic mobile phase CHCl₃/CH₃OH/H₂O/CH₃COOH (73/27/4/1). The fractions (tR=35 to 37 minutes) that contained the major product were combined and evaporated to remove the solvent, dried under high vacuum for 24 h to obtain 35.5 mg (26.5%) of the desire product (12). ¹H-NMR (CDCl₃/CD₃OD(1/1)): δ 7.74 (6H, bm, Ar—H), 6.96 (6H, bm, Ar—H), 4.59, 4.20, 3.80, 3.61, 3.32, 2.68, 2.20, 2.05, 1.94, 1.42 (overlapping peaks, 167H, bm, all CH and CH₂), 0.84 (6H, t, CH₃). High resolution MALDI-FTMS: m/z 2681.4711 (calcd for C₁₃₀H₂₀₉N₂₅O₂₉S₃+H, m/z 2681.4882)

### Example 3

### Preparation of [(PDA-PEG3)₂-DTPA-(CONHPM)₃] (12), Lipid Chelator Conjugated to Integrin Agonist

DTPA-(COOH)₃ (11, 69 mg, 50 μmole) was dissolved in anhydrous CH₃CN (5 mL), anhydrous CH₂Cl₂ (2 mL) and Et₃N (1 mL) in a 3-neck round bottomed flask, previously flame dried and filled with argon. To this solution was added the BOP reagent (134 mg, 150 μmol) and the solution was stirred well for 5 minutes. A solution of 10 (69 mg, 150 μmol) was prepared in a dry vial filled with argon, in a mixture of anhydrous CH₃CN (5 mL) and anhydrous DMF (2 mL). The solution of 10 was added to the lipid solution using a dry syringe a with continuous stirring. The reaction was allowed to stir for 10 hours in dark. TLC solvent: CHCl₃, CH₃OH, H₂O, and CH₃COOH showed complete disappearance of the starting material (R_{f}=0.53). There was one major product (R_{f}=0.2) and 5 minor products (R_{f}<0.18). The solvent was removed by spin evaporation and dried under high vacuum for 24 hours. The crude product was purified by normal phase HPLC using a semi preparative silica column, flow rate 5 mL/min. Gradient system starting with 100% CHCl₃ for 5 min, then 75% CHCl₃/25% CH₃OH for 10 minutes, then 50% CHCl₃/50% CH₃OH for 10 minutes, then 25% CHCl₃/75% CH₃OH for 10 minutes, and finally for 20 minutes with 100% CH₃OH. The fractions t_{R}=35 to 37 minutes) that contained the major product were combined and spin evaporated to remove the solvent, dried under high vacuum for 24 hours to obtain 35.5 mg (26.5%) of the desired product. High resolution MALDI-FTMS: m/z 2681.4711 (calculated for C₁₃₀H₂₀₉N₂₅O₂₉S₃+H, m/z 2681.4882)

### Example 4

### Preparation of Paramagnetic Polymerized Nanoparticles (PV1 through PV6).

Appropriate amounts of purified lipid components (12, 13, 14, and 15) dissolved in organic solvents (CHCl₃ and CH₃OH in a ratio 1:1) were combined. The solvents were evaporated and the residue dried in vacuo for 24 h while shielded from light. Distilled and deionized water was added to yield a heterogeneous solution 30 mM in lipid concentration. The lipid/water mixture was then sonicated with a probe-tip sonicator for at least one hour and the solution became clear. Throughout sonication, the pH of the solution was maintained between 7.0 and 7.5 with 1N NaOH solution, and the temperature was maintained above the gel-liquid crystal phase transition point (Tₘ) with the heat generated from sonication. To polymerize the liposomes, the liposome solution was transferred to a petri dish resting on a bed of wet ice, cooled to 0° C., and irradiated at 254 nm for at least one hour with a hand-held UV lamp placed −1 cm above the petri dish, yielding PVs. The PVs were then filtered through a 0.2 μm filter and collected. Composition and physical properties of the PVs are shown in Table Z:

### Example 5

### Polymerized Nanoparticle Binding to Integrin

In order to assess the unility of the PV's in targeting integring, PVs were constructed as outlined in Table Z and labeled with europium. Integrin binding was determined by coating purified αᵥβ₃ onto 96 well plates and then PVs were added with incubated at room temperature. The unbound PVs were removed by washing with buffers and the bound PVs were measured using time resolved fluorescence of the europium in the PV's (Wallac, Gaitherburg, Md. 20877 USA). The materials that contained 10 mol % of compound 12, (PV1 and PV5) had the highest avidity for the integrin αᵥβ₃.

In a competitive integrin binding assay, the PVs (PV1-PV5) were mixed with various concentrations of 10 to inhibit 50% of binding of the PVs to αᵥβ₃. The reported values are average of quadruplicate values and have a maximum standard error ±3. A schematic of this assay is shown in FIGS. 18A-18B.

### Example 6

### In vitro Assay for the Inhibition of Cell Adhesion

A cell adhesion inhibition study was done on plates coated with vitronectin (Wu, et al., In Methods in Molecular Biology: Integrin Protocols; Howlett, Ed.; Humana Press: Totowa, N.J., 1999; vol. 129, pp 211-217), using a human melonoma cell line M21. The multivalent particle complex PV1-PV6 as well as the monomeric ligand 10 were separately incubated with M21 cells and applied onto the 48 well plates coated with vitronectin. After 1 h incubation, the wells were washed and the cells that adhered were stained with a solution of crystal violet and the OD at 590 nm was measured. The OD measured was proportional to the number of cells bound to the vitronectin plate and was plotted against the concentration of 10 on the surface of the PVs in different formulations to calculate the IC₅₀. The reported values are average of quadruplicate values and have a maximum standard error of 0.05.

The multivalency effect was calculated by dividing the IC₅₀ for free ligand 10 by the IC₅₀ of the concentration of 10 on the PVs.

### Example 7

### Use of Paramagnetic Polymerized Nanoparticles in Imaging Tumors in vivo

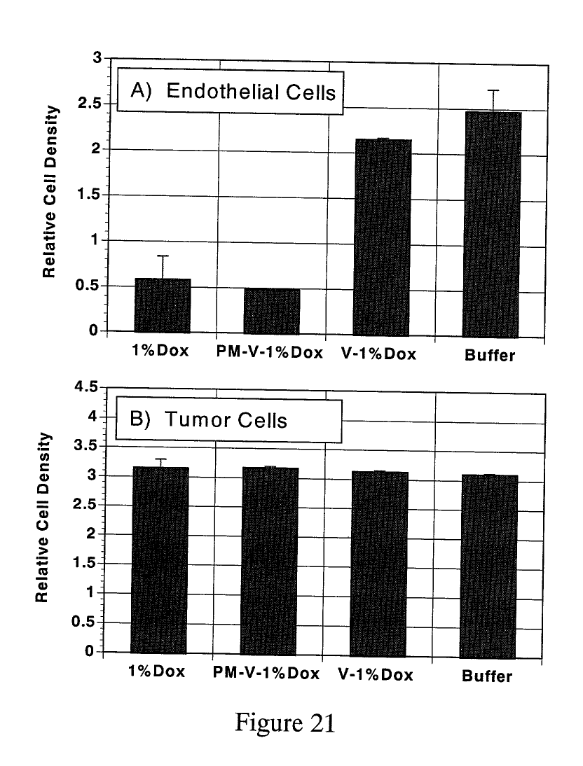

C3H/Km mice aged 10 to 12 weeks were anesthetized (Nembutal (58 mg/kg)), and their right flanks were shaved and an average of 2×10⁵ tumor cells (mouse MK504 melanoma cells) in Hanks' solution (0.5 mL) were injected intradermally in the right flank region of each mouse with a 27 G needle. Mice were monitored for tumor growth. Approximately 2 weeks were required for tumors to grow to 1 cm in size. Two mice with tumors were divided into two groups. Both the PVs were labeled with radioactive indium (¹¹¹In) as previously described, Storrs, et al., 1995a; Haubner, et al., Cancer Res. (2001) 61:1782-1785, and then were administered to the mice via tail vein injection (0.1 mg of lipid/gram weight of the animal (g); 7.1 μg/g of 10 on the surface of the PVs; 12.5 μCi/g). Using gamma scintigraphy, the accumulation of PV1 in the tumor was approxmately 5%±1% of the total counts observed in the animal. Group 1 was treated with intravenous nanoparticle-integrin antagonist complex PV1, and Group 2 received intravenous nanoparticle complex PV4. Results are shown in FIGS. 20A-20E. Those treated with PV4 showed no significant enhancement (<0.5% total counts) in the tumor (FIG. 20D-E). The PVs in the treatment group (PV1) showed enhancement in the tumor even after 24 hours (FIGS. 21B-C), indicating that multivalency gives rise to a stable complex of PV1 to the tumor site in vivo (see supplementary material). These results demonstrate that multivalent PVs can be used in vivo to target tumors and the material is retained in the tumor even after 24 hours. These materials can, therefore, serve as spatial and temporal imaging agents that have high avidity for the integrins in vivo.

### Example 8

### Preparation of Integrin-Targeting Liposomes Containing an Integrin-Targeting Lipid and Ammonium Sulfate

BisT-PC 13 (500 mg, 546.9 μmole, 95 mole %) was weighed into a clean 100 ml round bottom flask. Chelator lipid 15 (3.15 ml, 31.5 mg, 23 μmole, 4 mole %), and RGD peptidomimetic lipid 12 (1.54 ml, 15.4 mg, 5.74 μmole, 1 mole %) were added to the flask by glass syringe. Chloroform was removed by rotary evaporation. The lipid film was hydrated with 20 ml of 250 mM ammonium sulfate and 190 μl 0.5 N NaOH while rotating the flask in the 65° C. water bath. Immediately prior to extrusion, the lipid suspension was briefly sonicated in the 100 ml flask to reduce the size of the aggregates and then transferred to the extruder. The lipid suspension was extruded through a series of successively smaller pore size polycarbonate (PC) membranes. The 10 ml thermal barrel extruder maintained at 90° C. was fitted with 2 stacked membranes and the lipid suspension was extruded through 100 nm membranes, then 50 nm membranes, and finally 30 nm membranes using argon at 300-600 p.s.i. The vesicles were transferred to dialysis cassettes and dialyzed against 10% sucrose (2×1800 ml, 4 h). The size determined by dynamic light scattering was approximately 60 nm.

### Example 9

### Preparation of Integrin-Targeting Liposomes Containing an Integrin-Targeting Lipid and Sodium Citrate

BisT-PC 13 (91.4 mg, 99.96 μmoles, 95 mole %) chelator lipid 15 (5.8 mg, 4.24 μmoles, 4 mole %), and RGD peptidomimetic lipid 12 (2.8 mg, 1.04 μmoles, 1 mole %) were added to a 100 mL flask and dissolved in 10 ml of chloroform. Chloroform was removed by rotary evaporation for 60 minutes at 65° C., and 10 ml of 0.3 M sodium citrate at pH 4 was added to the evaporated lipid. The heterogeneous solution was frozen on acetone/dry ice and thawed in a 65° C. water bath. This process was repeated four times, and the solution was extruded three times in a thermal barrel extruder at 65° C. through two 0.1 μm filters, followed by extrusion six times through two 0.05 μm filters. The size determined by dynamic light scattering was 80 nm.

### Example 10

### Preparation of Non-Targeting Liposomes Containing Ammonium Sulfate

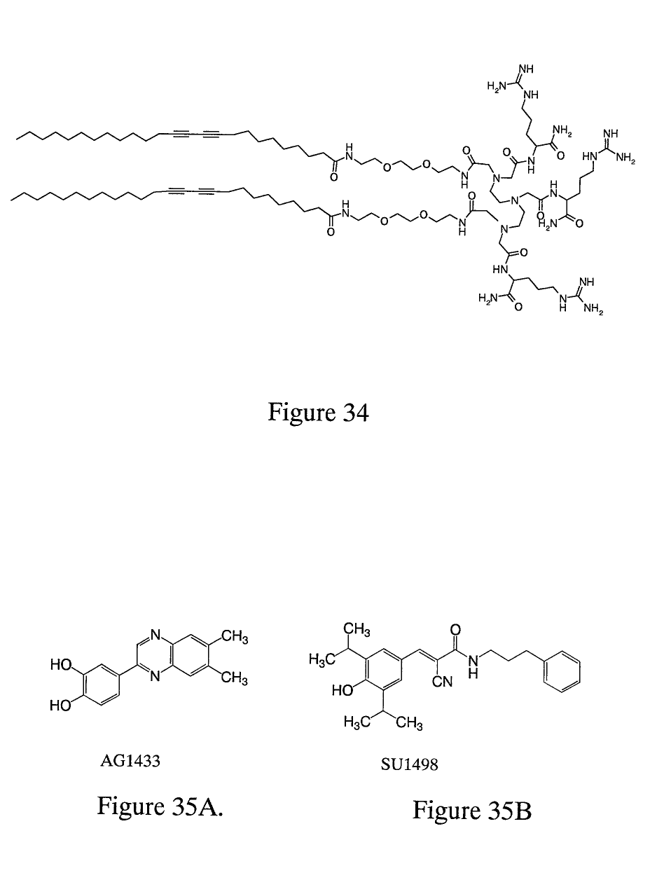

Non-targeting liposomes were prepared exactly as described in EXAMPLE 8, except no integrin-targeting lipid was used, and the mole percent of chelator lipid 15 was 5%. Alternatively, non-targeting vesicles containing 1 mole % of the tri-arginine lipid 18 (FIG. 34) were prepared exactly as described in EXAMPLE 8, except the RGD peptidomimetic lipid 12 was omitted.

### Example 11

### Preparation of Integrin-Targeted Vesicles Containing 10% Doxorubicin

Integrin-targeted liposomes containing ammonium sulfate from EXAMPLE 8 (2 mL, 60 mg) were placed in a 12×100 mm glass culture tube and 600 μl (6 mg) doxorubicin in 10% sucrose was added. The mixture was incubated for 5 minutes at 65° C. and size exclusion chromatography (SEC) showed that the loading of doxorubicin was quantitative. SEC analysis was performed with 10 mM HEPES buffer containing 200 mM NaCl pH 7.4 by adding a 100 μl sample from the doxorubicin loading mixture to a Sepharose CL 4B column (1.5×6 cm). The mixture was diluted with 10% sucrose to give a final vesicle concentration of 15 mg/ml. These vesicles contain 10% doxorubicin by weight. The size measured by dynamic light scattering was 60-65 nm.

### Example 12

### Preparation of Integrin-Targeted Vesicles Containing 1% Doxorubicin

Integrin-targeted liposomes containing ammonium sulfate from EXAMPLE 8 (2 mL, 60 mg) were placed in a 12×100 mm glass culture tube and 60 μl (0.6 mg) doxorubicin solution added. The tube was immersed in a water bath maintained at 65° C. for 5 minutes. The mixture was diluted with 10% sucrose to give a final vesicle concentration of 15 mg/ml. SEC analysis was performed as described in EXAMPLE 11 and showed that all doxorubicin added was encapsulated in the liposome. These vesicles contain 1% doxorubicin by weight. The size measured by dynamic light scattering was 60-65 nm.

### Example 13

### Preparation of Integrin-Targeted Vesicles Containing 20% Doxorubicin

The solution of the integrin-targeted vesicles containing citrate from EXAMPLE 9 was adjusted to pH 8 with 1 M HEPES buffer at pH 7.4 and sodium hydroxide. To this solution was added 200 μl of doxorubicin (10 mg/ml in 10% sucrose) to 1 ml (10 mg) of vesicles at pH 8 and the solution was incubated for 7 min at 65° C. in a water bath. SEC analysis was performed as described in EXAMPLE 12 and showed that all doxorubicin added was encapsulated in the liposome. The size measured by dynamic light scattering was 93 nm.

### Example 14

### Preparation of Non-Targeting Liposomes Containing 10% Doxorubicin

Vesicles (60 mg, 2 ml) from EXAMPLE 10 were placed in a 12×100 mm glass culture tube and 600 μl doxorubicin solution (10 mg/ml in 10% sucrose) was added. The tube was immersed in a water bath maintained at 65° C. for 5 minutes. The mixture was diluted with 10% sucrose to give a final vesicle concentration of 15 mg/ml. SEC analysis was performed as described in EXAMPLE 12 and showed that all doxorubicin added was encapsulated in the liposome. These vesicles contain 10% doxorubicin by weight.

### Example 15

### Preparation of Vesicles Containing N-succinyl-DPPE and Ammonium Sulfate

BisT-PC (1 g, 1093.7 μmole, 95 mole %) and N-succinyl-DPPE (47 mg, 57.6 μmole, 5 mole %), were weighed into a clean 100 ml round bottom flask and dissolved in 20 ml chloroform. Chloroform was removed by rotary evaporation. The lipid film was hydrated with 40 ml 250 mM ammonium sulfate and 500 μl 0.5 N NaOH while rotating the flask in the 65° C. water bath. The pH after hydration was 7.5. Vesicles were prepared with a thermal barrel extruder at 65° C. by passing the solution through two stacked membranes with pore sizes of 100 nm (400 psi argon), then 50 nm membranes (400 psi argon), and finally 30 nm membranes (700 psi argon). The vesicles were transferred to dialysis cassettes and dialyzed against 10% sucrose. The size determined by dynamic light scattering was approximately 68 nm. This procedure was also used without the addition of sodium hydroxide to prepare vesicles containing 10 mole percent of the N-succinyl-DPPE lipid, 50 mole percent of dimyristoyl-sn-glycero-3-phosphocholine (DMPC), dipalmitoyl-sn-glycero-3-phosphocholine (DPPC), or distearoyl-sn-glycero-3-phosphocholine (DSPC), and 40 mole percent cholesterol.

### Example 16

### Preparation of Vesicles Containing N-succinyl-DPPE

Vesicles identical to those in EXAMPLE 15 were prepared without ammonium sulfate.

### Example 17

### Preparation of Aminodextran-Vesicle Conjugates

Vesicles prepared with 95 mole percent 1,2-bis(10, 12-tricosadiynoyl)-sn-glycero-phosphocholine (BisT-PC 13, Avanti Polar Lipids) and 5 mole percent of the DTPA lipid derivative 13 were coated with aminodextran as follows: Vesicles (10 ml, 250 mg) were added dropwise to stirred aminodextran (amine modified 10,000 MW dextran, Molecular Probes, product D-1860, 500 mg, 3 amino groups per dextran polymer) in 5 ml of 50 mM HEPES buffer at pH 8. EDAC (Aldrich 16146-2, ethyldimethylaminodipropyl carbodimimide HCl salt, 6 mg) in 200 μg of water was added dropwise to the coating mixture while stirring. The mixture was stirred at room temperature overnight. The clear reaction mixture was purified by size exclusion chromatography on a Sepharose CL 4B column (2.5×30 cm, Amersham Pharmacia Biotech AB product 17-0150-01) equilibrated with 10 mM HEPES containing 200 mM NaCl at pH 7.4. When the coated vesicles began to elute, 4 ml fractions were collected. The peak fractions (2 thru 6) were pooled and filtered through a 0.45 μm filter (Nalgene 25 mm syringe filter, product 190-2545) followed by a 0.2 μm filter (Nalgene 25 mm syringe filter, product 190-2520). The concentration of coated vesicle was determined by drying a sample to constant weight in an oven maintained at 90° C.

### Example 18

### Succinylation of Aminodextran-Vesicle Conjugates

Aminodextran-coated vesicles from EXAMPLE 17 (15 ml, 465 mg) in 10 mM HEPES buffer at pH 7.4 were diluted with an equal volume of 200 mM HEPES buffer and the pH was adjusted to 8 with 1 N NaOH. Succinic anhydride (Aldrich product 23,969-0, 278 mg) was dissolved in 1 ml DMSO (dimethyl sulfoxide (Aldrich product 27685-5) and 100 μl aliquots were added to the coated-vesicle suspension with rapid stirring. The pH was monitored and adjusted as necessary to maintain the pH between 7.5 and 8 by the addition of 1 N NaOH. After the final addition of succinic anhydride, the mixture was stirred for 1 hour at room temperature and then transferred to dialysis cassettes and dialyzed against 10 mM HEPES buffer at pH 7.4.

### Example 19

### Coupling of an RGD Peptidomimetic to Succinylated, Dextran-Vesicle Conjugates

The succinylated aminodextran-coated vesicles from EXAMPLE 18 (200 mg in 6.9 ml water) and αᵥβ₃ integrin-targeting agent 12 (40 mg in 1 ml of water) were mixed with water (6.1 ml), 1 M NaCl (3 ml) and 500 mM MES buffer pH 6 (2 ml). EDAC (19.2 mg, 1 ml) was added. The solution was mixed and incubated at room temperature for 18 h. Analysis of the reaction mixture by size exclusion chromatography showed that the coupling yield was approximately 30-50%. The conjugate was dialyzed twice in a 10K MWCO cassette in 3.5 L of 50 mM histidine buffer containing 5 mM citrate at pH 7.4 for 8 and 18 h.

### Example 20

### Preparation of Lipid-Based, Integrin-Targeted Particles Containing Paclitaxel

Integrin-targeted paclitaxel particles containing integrin-targeting lipid 12 are made as described in EXAMPLE 8, but without ammonium sulfate. For example, the preparation of 100 mg of vesicles containing BisT-PC 13, chelator lipid 15, integrin-targeting lipid 12, and 4.5% w/w paclitaxel was achieved using 90.7 mg BisT-PC, 6.5 mg PDA-DTPA, 2.8 mg integrin-targeting lipid, and 4.5 mg paclitaxel. HPLC analysis also showed that this process did not result in the degradation of paclitaxel, and the size was 63 nm.

### Example 21

### Preparation of RGD Peptidomimetic Vesicles Containing Paclitaxel and DMPC

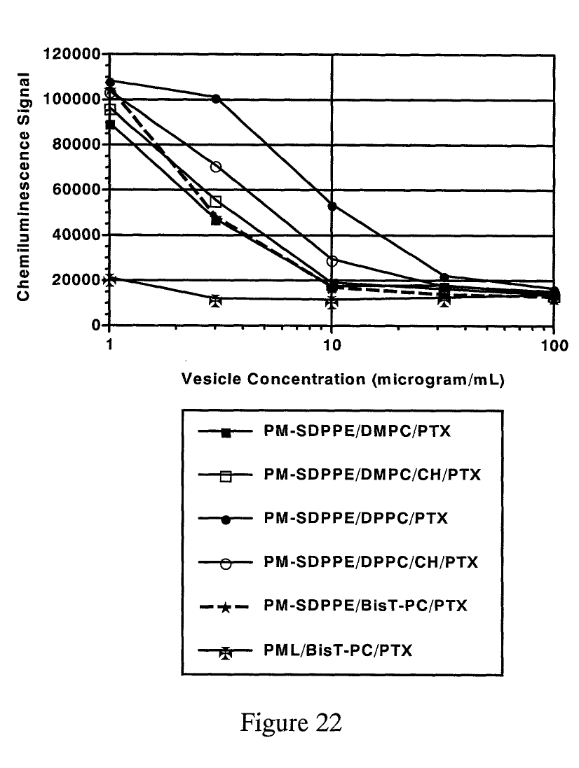

The following procedure can be used to prepare vesicles containing 1-10 weight percent paclitaxel. DMPC in chloroform (42.5 mg, 62.7 umole; Avanti), N-succinyl-DPPE in 1:1 chloroform/methanol (5 mg, 6.1 umol; Avanti), and paclitaxel in chloroform (2.5 mg, 2.9 μmole; Sigma) were placed in a round bottom flask. The total volume was 5 mL. The solvent was removed at 48° C. by rotory evaporation. The vacuum-dried lipid was hydrated with 5 ml of 50 mM HEPES buffer pH 7.4 while mixing in a 48° C. water bath. The mixture was extruded through a Lipex 10 ml thermal barrel extruder at 48° C. using 50 nm polycarbonate track-etched filters (Osmonics) by applying 700 psi of pressure of argon. The process was repeated 5 times, followed by extrusion 5 times through 50 nm filters. The size measured by dynamic light scattering was 73 nm. RGD peptidomimetic 10 was attached to the vesicles containing taxol by activation of the carboxyl group of the N-succinyl-DPPE lipid in the vesicles with EDC in the presence of the peptidomimetic. Alternatively, the vesicles may be activated with EDC, followed by the addition of the peptidomimetic, or the vesicles may be activated with EDC, followed by removal of remaining EDC by size exclusion chromatography, followed by the addition of the peptidomimetic to the activated vesicles. In a typical procedure, Vesicles (15 mg, 1 mM carboxyl group), peptidomimetic 10 (2 mM) and EDC (5 mM) are incubated in a volume of 1.5 mL at room temperature in a 1.5 mL polypropylene tube. The conjugate was dialyzed against 50 mM HEPES buffer at pH 7.4 (10K MWCO dialysis cassette) to remove unreacted peptidomimetic. The attachments were monitored by SEC analysis, and the RGD peptidomimetic-vesicle conjugates containing paclitaxel inhibit the binding of biotinylated fibrinogen, as shown in FIG. 22.

### Example 22

### Preparation of RGD Peptidomimetic Vesicles Containing Paclitaxel DMPC, and Cholesterol

Vesicles identical to those in EXAMPLE 21 were prepared, except the components were DMPC (30.8 mg, 45.4 umole), N-succinyl-DPPE (5 mg, 6.1 umol; Avanti), cholesterol (11.7 mg, 30.3 umol), and paclitaxel (2.5 mg, 2.9 μmole; Sigma). The size measured by dynamic light scattering was 85.3 nm.

### Example 23

### Preparation of RGD Peptidomimetic Vesicles Containing Paclitaxel and DPPC

Vesicles identical to those in EXAMPLE 21 were prepared, except the components were DPPC (42.5 mg, 57.9 umole), N-succinyl-DPPE (5 mg, 6.1 umol; Avanti), and paclitaxel (2.5 mg, 2.9 μmole; Sigma). The size measured by dynamic light scattering was 80.0 nm.

### Example 24

### Preparation of RGD Peptidomimetic Vesicles Containing Taxol, DPPC and Cholesterol

Vesicles identical to those in EXAMPLE 21 were prepared, except the components were DPPC (31.4 mg, 42.8 umole), N-succinyl-DPPE (5 mg, 6.1 umol; Avanti), cholesterol (11.1 mg, 28.6 umol), and paclitaxel (2.5 mg, 2.9 μmole; Sigma). The size measured by dynamic light scattering was 91.1 nm.

### Example 25

### Preparation of RGD Peptidomimetic-Dextran-Vesicle Conjugates Containing Doxorubicin by Process 1

A dried lipid film containing BisT-PC (1 g, 1093.7 μmole, 95 mole %) and N-succinyl-DPPE (47 mg, 57.6 μmole, 5 mole %) was prepared by rotary evaporation of a chloroform solution. The dried film was hydrated by addition of 250 mM ammonium sulfate and warming in a 65° C. water bath for 30 minutes. The hydrated lipid suspension was then extruded through a series of successively smaller pore sized polycarbonate track etched filter membranes using a thermal barrel extruder maintained at 65° C. Extrusion was initiated with a 100 nm pore size filter and terminated with a 30 nm pore size filter. Excess ammonium sulfate was removed by dialysis in 10% sucrose solution. The vesicles were coated with aminodextran, succinylated, and coupled to integrin antagonist 10 by the procedure described in Examples 17-19. Doxorubicin was loaded into the vesicles by mixing with a sucrose solution of doxorubicin and warming the mixture to 65° C. for 5 minutes. In a typical preparation, doxorubicin at 10 mg/mL in 10% sucrose solution was added to 1 mL of vesicles containing ammonium sulfate. Complete uptake of the added doxorubicin was confirmed by SEC on a column of Sepharose CL 4B equilibrated and eluted with 10 mM HEPES, 200 mM NaCl pH 7.4.

### Example 26

### Preparation of RGD Peptidomimetic-Dextran-Vesicle Conjugates Containing Doxorubicin by Process 2

Succinylated dextran-coated vesicles containing BisT-PC (1 g, 1093.7 μmole, 95 mole %) and N-succinyl-DPPE were prepared as described in EXAMPLE 20, except no ammonium sulfate was used. The RGD mimetic 10 was coupled to these vesicles as described in EXAMPLE 19. The resulting RGD mimetic-dextran vesicle conjugates were suspended in 250 mM ammonium sulfate solution and heated to 65° C. for 30 minutes. Excess ammonium sulfate was removed by dialysis with 10% sucrose solution. Doxorubicin was loaded into the vesicles by mixing with a sucrose solution of doxorubicin and warming the mixture to 65° C. for 5 minutes. In a typical preparation, doxorubicin at 10 mg/ml in 10% sucrose solution was added to 1 ml of vesicles containing ammonium sulfate. Uptake of the added doxorubicin was confirmed by SEC on a column of Sepharose CL 4B equilibrated and eluted with 10 mM HEPES, 200 mM NaCl pH 7.4.

### Example 27

### Preparation of Integrin-Targeted Liposomes Containing Doxorubicin

The αᵥβ₃ integrin-binding RGD peptidomimetic 10 was attached to liposomes containing ammonium sulfate (EXAMPLE 15) using the method described in EXAMPLE 19. For example, the peptidomimetic was attached in 50 mM HEPES buffer at pH 7 to ammonium sulfate loaded vesicles containing N-succinyl-DPPE, DMPC, and cholesterol in mole ratios of 10/50/40 by adding EDAC to a final concentration of 5 mM, followed by 2 equivalents of the peptidomimetic 10 to generate vesicles containing approximately 14 μg of the peptidomimetic per mg of lipid.

### Example 28

### Attachment of ⁹⁰Y to Peptidomimetic-Vesicle Complexes

The peptidomimetic-vesicle complexes containing chelator lipid 15 are labeled with ⁹⁰Y in 50 mM histidine buffer containing 5 mM citrate at pH 7.4 by the following procedure. Yttrium-90 chloride in 50 mM HCl (NEN Life Science Products) was diluted to a working solution containing approximately 20 mCi/mL. To 100 μL of the Integrin-targeted vesicles (0.1-50 mg/mL), approximately 100-250 μCi of yttrium-90 chloride (NEN Life Science Products) was added, mixed using a vortex mixer, and incubated at room temperature for 30 minutes. In duplicate, the percent ⁹⁰Y bound to the therapeutic vesicle was determined by adding 100 μL of the ⁹⁰Y-vesicle complex to a 100k MWCO Nanosep™ (Pall Filtron) filter. The filter assembly was spun in a microfuge at 4000 rpm for 1 hr or until all of the solution has passed through the filter. The “total ⁹⁰Y” in the assembly was determined with the Capintec CRC-15R dosimeter. The filter portion of the assembly was removed and discarded. Using the dosimeter, the remaining part of the assembly containing the “unbound 90Y” that passed through the filter was counted. “Bound ⁹⁰Y” was determined by subtracting the “unbound ⁹⁰Y” from the “total ⁹⁰Y”. Percent⁹⁰Y bound was determined by dividing the “bound ⁹⁰Y” by the “total ⁹⁰Y” and multiplying by 100.

### Example 29

### Study of Antitumor Efficacy of ⁹⁰Y-peptidomimetic-Vesicle Complexes in a Mouse Melanoma Model

The K1735-M2 mouse melanoma model was prepared by subcutaneous injection of tumor cells as previously described (X. Li, et al. Invasion Metastasis 1998, 18, 1-14). Animals received a single i.v. injection of placebo or therapeutic agent and tumor volume was measured until the tumors had quadrupled in size. Tumors were induced in the mice as follows: tumors were implanted by subcutaneous injection of approximately 1×10⁶ K1735 M2 melanoma cells (X. Li, B. Chen, S. D. Blystone, K. P. McHugh, F. P. Ross, D. M. Ramos, Differential expression of alpha v integrins in K1735 melanoma cells. Invasion Metastasis 18(1) (1998) 1-14). The K1735 M2 tumor cells were grown in tissue culture flasks in Dubelco's medium with 10% fetal calf serum (FCS). Cells were harvested using Trypsin-EDTA solution (containing 0.05% trypsin), resuspended in PBS at 10,000,000/ml, and kept on ice. Animals with tumors between 100 and 200 mm³ were selected for treatment as described in Table I.

TABLE I
Description of treatment groups

| | # | IA Dose* | NP Dose* | Y90 Dose* |
|---|---|---|---|---|
| Group | Animals | (μg/g) | (mg/g) | (μCi/g) |
| 1) Buffer | 8 | NA | NA | NA |
| 2) IA | 8 | 14 | NA | NA |
| 3) IA-NP | 8 | 14 | 0.1 | NA |
| 4) NP-Y90 (low) | 8 | NA | 0.1 | 2.5 |
| 5) NP-Y90 (high) | 8 | NA | 0.1 | 5.0 |
| 6) IA-NP-Y90 (low) | 8 | 14 | 0.1 | 2.5 |
| 7) IA-NP-Y90 (high) | 8 | 14 | 0.1 | 5.0 |

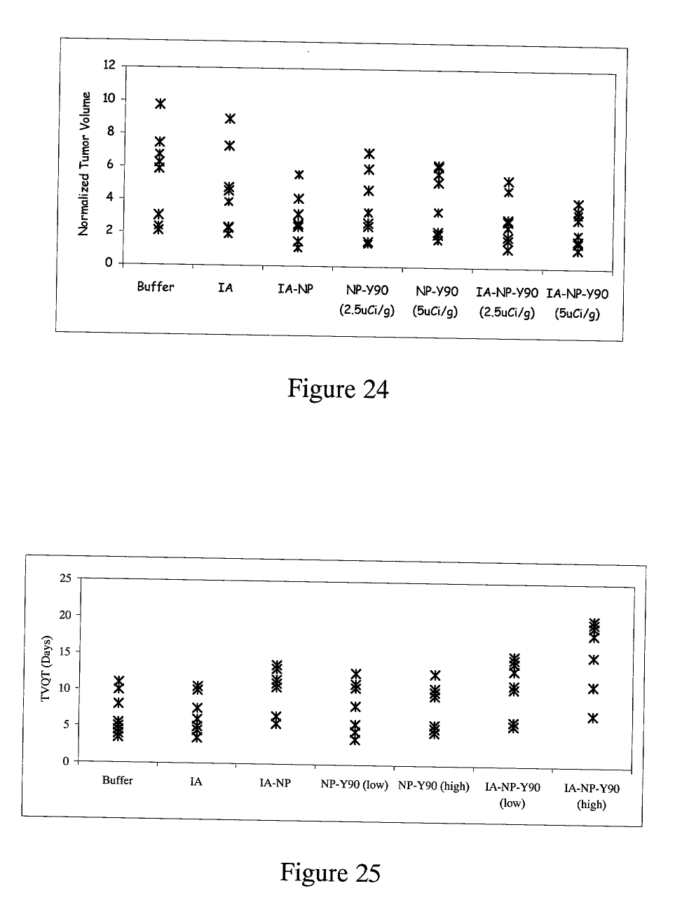

FIG. 23 shows the normalized tumor volume data obtained in this study. The seventh day post treatment is the last day that all animals in the study were alive. FIG. 24 on the following page shows the normalized tumor volumes for each animal sorted by treatment group on the seventh day post treatment.

Normalized tumor volume seven days post treatment was compared using analysis of variance (ANOVA) and Kruskal-Wallis statistical tests. These tests determine if the observed differences between treatment groups are due to chance alone. The ANOVA tests the equality of the treatment means. The ANOVA is most reliable when there are no significant outliers in the data. The Kruskal Wallis test, on the other hand, considers the order, or rank of the tumors in a given group compared to other treatments and therefore minimizes the impact of outliers. The Kruskal-Wallis test looks for significant differences in the medians of the treatment populations and is more reliable when the data contains significant outliers.

For normalized tumor volume seven days post treatment, the P-value for the ANOVA test was 0.052. The P-value for the Kruskal-Wallis test was 0.167. Neither of these tests is significant at the 95% confidence level. As FIG. 23 and FIG. 24 show, this study contains a number of control groups with small differences in efficacy. In order to determine if the number of treatment groups diluted the results of the statistical tests the tests were repeated after removing some of the less distinct control groups. When only buffer, IA-NP, IA-NP-Y90 (2.5) and IA-NP-Y90 (5) treatments are compared the P-values improve substantially (0.009 for the ANOVA test and 0.034 for the Kruskal-Wallis test). This indicates that there is at least one significantly different treatment in this reduced comparison.

Pairwise, or one to one, comparisons of the different treatment groups were made with different statistical procedures. The results indicate that IA-NP and IA-NP-Y90 (2.5) treatments when compared to treatment with buffer may have significantly lower normalized tumor volumes depending on the how the data are analyzed. On the other hand, treatment with IA-NP-Y90 (5) compared to treatment with buffer yields significantly lower normalized tumor volume regardless of the statistical test employed.

Tumor growth delay was also used to monitor efficacy in this study. Tumor growth delay is defined as the time required for a given tumor to show a fourfold increase in volume when compared with the tumor volume measured on the day of treatment (Tumor Volume Quadrupling Time or TVQT). The exact time for four-fold growth is extrapolated by drawing a line between the two nearest time points. FIG. 23 summarizes the growth delay data for this study.

Again ANOVA and Kruskal-Wallis tests were used to compare TVQT values from different treatment groups. The P-values associated with both tests were highly significant (0.001 for the Kruskal-Wallis test and <0.0005 for the ANOVA). Pairwise comparisons of the different treatment groups indicate that treatment with IA-NP-Y90 at higher radiation doses (5 μCi./g) is significantly different from buffer, IA and both low and high IA-NP treatments. Table III on the following page shows the results of Tukey's W pairwise comparison procedure. These results were confirmed by non-parametric statistical tests as well.

TABLE III
Summary of P-values obtained using Tukey's pairwise
comparisons with tumor volume quadrupling time data.

| | | | | NP-Y90 | NP-Y90 | IA-NP-Y90 |
|---|---|---|---|---|---|---|
| | Buffer | IA | IA-NP | (2.5 μCi/g) | (5 μCi/g) | (2.5 μCi/g) |
| IA | >0.05 | | | | | |
| IA-NP | >0.05 | >0.05 | | | | |
| NP-Y90 (2.5 μCi/g) | >0.05 | >0.05 | >0.05 | | | |
| NP-Y90 (5 μCi/g) | >0.05 | >0.05 | >0.05 | >0.05 | | |
| IA-NP-Y90 (2.5 μCi/g) | >0.05 | >0.05 | >0.05 | >0.05 | >0.05 | |
| IA-NP-Y90 (5 μCi/g) | <0.01 | <0.01 | >0.05 | <0.01 | <0.01 | >0.05 |

Eight days post treatment one tumor from each treatment group was selected at random for histological staining. The tumors were resceted and frozen in isopentane at liquid nitrogen temperatures.

Marin Biologic Laboratories, Inc. in Tiburon Calif. performed TUNEL assay, Von Willebrand's Factor and H&E staining on resceted tumors. TUNEL assay results indicate that Hist/Cit Buffer, IA, and NP-Y90 2.5 μCi/g treatment result in mostly healthy cells, while, IA-NP, IA-NP-Y90 2.5 μCi/g, and IA-NP-Y90 5 μCi/g, show inceasing amounts of apotosis and cell death.

Treatment with IA-NP-Y90 at 5 μCi/g significantly reduces tumor growth in this tumor model (significance was established at the 95% confidence level). On average the norrnalized tumor volume for tumors treated with IA-NP-Y90 at 5 μCi/g were less than half the volume when compared to tumors treated with buffer. In addition the average TVQT for tumors treated with IA-NP-Y90 at 5 μCi/g is 15.0 days compared to 6.4 days for tumors treated with buffer. Histological study of tumor samples confirms this result.

Interestingly, melanoma cells are known to be relatively resistant to radiotherapy. This type of targeted therapy relies only on the presence of neovascular cell surface markers on the endothelial cells that are terminally differentiated and genetically stable.

### Example 30

### Study of Antitumor Efficacy of Peptidomimetic-Dextran-Vesicle ⁹⁰Y Complexes in a Mouse Melanoma Model

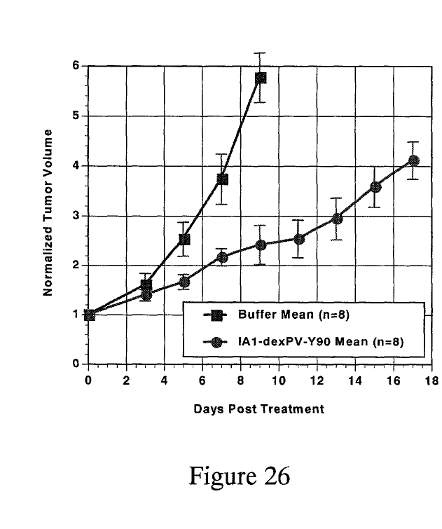

Dextran coated vesicles were also tested in the mouse melanoma model as described in EXAMPLE 29. Results are shown in FIG. 26. For these studies, dextran-coated vesicles containing BisT-PC and chelator lipid 15 were used, and they were prepared as described in Examples 17-19, and labled with yttrium-90 as described in Example 28.

### Example 31

### Study of Antitumor Efficacy of Peptidomimetic-Vesicle-⁹⁰Y Complexes in a Mouse Colon Cancer Model

In this study, a CT-26 colon cancer cell line, implanted by subcutaneous injection in female BALB/c mice as previously reported (H. N., Moehler, T., Siang, R., Jonczyk, A., Gillies, S. D., Cheresh, D. A., Reisfeld, R. A., Proc. Natl. Acad. Sci. USA, 96: 1591-1596, 1999), was used to assess the anti-tumor activity of Targesome's radiopharmaceutical agent. The purpose of this study was to investigate the potential anti-tumor effects with a single intravenous administration of the IA-NP-Y90 complex.

Tumors were implanted by subcutaneous injection of approximately 1×106 CT-26 cells. The CT-26 tumor cells were grown in tissue culture flasks in Dulbeco's medium with 10% fetal calf serum (FCS). Cells are harvested using Trypsin-EDTA solution (containing 0.05% trypsin), resuspended in PBS at 10,000,000/ml, and kept on ice.

TABLE I
Description of treatment groups

| | IA dose | NP dose | **Y90 dose | Number |
|---|---|---|---|---|
| Group | (μg/g) | (mg/g) | (μCi/g)*** | of mice |
| 1) Buffer | NA | NA | NA | 8* |
| 2) IA | 13.7 | NA | NA | 8 |
| 3) IA-NP | 13.7 | 0.1 | NA | 8* |
| 4) NP-Y90 | NA | 0.1 | 6 | 8* |
| 5) IA-NP-Y90 | 13.7 | 0.1 | 6 | 8* |
| | | | Total | 38 |

*8 days post treatment one mouse was sacrificed for histological study from all but the IA groups.
**0.1 mg/g = 100 mg/kg
***6 μCi/g = 6 mCi/kg (6 times rabbit dose)

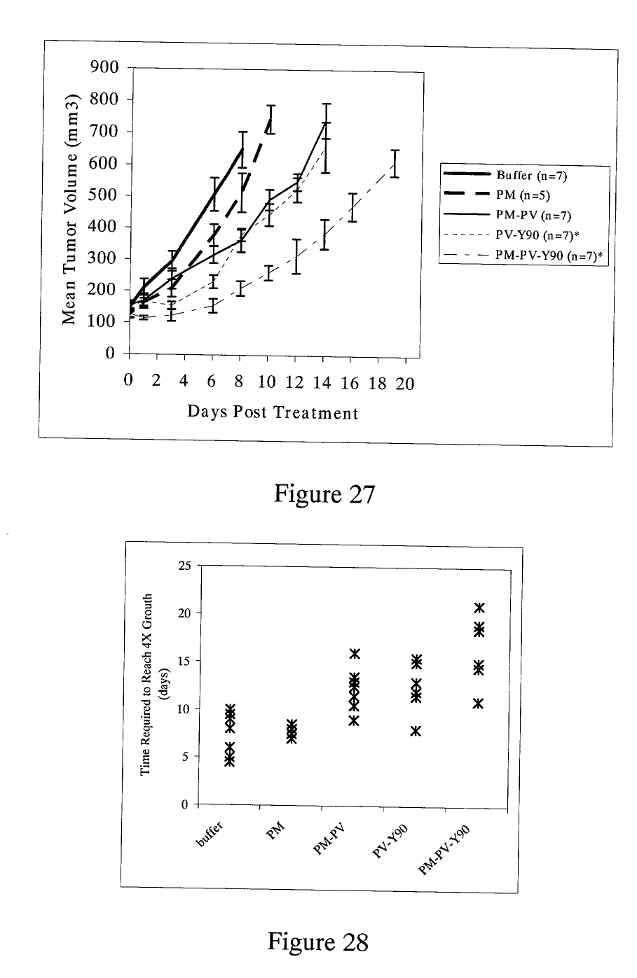

FIG. 27 summarizes the normalized tumor volume data. Day eight is the last day that all animals in the study were still alive. Differences between treatment groups were compared using analysis of variance (ANOVA) and Kruskal-Wallis statistical tests. In the case of the normalized tumor volume on day 8, the P-value for both the ANOVA and the Kruskal-Wallis tests is below 0.0005. It is reasonable to conclude that there are significant differences between treatments in this study. None of the treatment groups contained large outliers that might skew the results of an ANOVA analysis. For this reason Tukey's W procedure was used to determine which treatments show significantly different normalized tumor volumes on the eighth day post treatment.

TABLE III
Summary of P-values obtained using Tukey's Pairwise
Comparisons with Normalized Tumor Volume Measurements
Eight Days Post treatment.

| | Buffer | IA | IA-NP | NP-Y90 | IA-NP-Y90 |
|---|---|---|---|---|---|
| IA | >0.05 | | | | |
| IA-NP | <0.001 | 0.0183 | | | |
| NP-Y90 | 0.0021 | >0.05 | >0.05 | | |
| IA-NP-Y90 | <0.001 | <0.001 | >0.05 | >0.05 | |

As Table III indicates there is a significant difference in normalized tumor volume eight days post treatment between the following therapies:

Buffer compared with IA-NP, NP-Y90 or IA-NP-Y90

IA compared with IA-NP or IA-NP-Y90

Tumor growth delay was also used to monitor efficacy in this study. Tumor growth delay is defined as the time required for a given tumor to show a four-fold increase in volume when compared with tumor volume measured on the day of treatment. The exact time for fourfold growth is extrapolated by drawing a line between the two nearest time points. FIG. 28 summarizes the growth delay data for this study.

P-values associated with the ANOVA and Kruskal-Wallis tests were both less than 0.0005 indicating that the differences between treatment groups shown in FIG. 28 are not due to chance alone. Since there are no outliers in the tumor growth delay data, Tukey's Pairwise Comparison procedure was used to determine which treatments are significantly different from others. Table IV below shows the P-values obtained with Tukey's procedure.

TABLE IV

| | Buffer | IA | IA-NP | NP-Y90 | IA-NP-Y90 |
|---|---|---|---|---|---|
| IA | >0.05 | | | | |
| IA-NP | 0.0106 | 0.0400 | | | |
| NP-Y90 | 0.0103 | 0.0363 | >0.05 | | |
| IA-NP-Y90 | <0.001 | <0.001 | 0.0432 | >0.05 | |

As Table IV indicates there is a significant difference in tumor growth between the followingvtherapies:

Buffer compared with IA-NP, NP-Y90 and IA-NP-Y90

IA compared with IA-NP, NP-Y90 and IA-NP-Y90

IA-NP compared with IA-NP-Y90 (note, the significance value associated with this comparison is much lower than for the other significant comparisons). Interestingly, this tumor type is known to be resistant to radiation therapy. In vitro αᵥβ₃ integrin binding assay Integrin binding of RGD peptidomimetic-liposome conjugates containing the chelator lipid 15 was demonstrated in vitro using a radiometric binding assay. Briefly, 96-well plates coated with the αᵥβ₃ integrin were blocked with BSA. Samples of rabbit serum or buffer containing 0-100 micrograms/mL of the agonist-liposome-⁹⁰Y complex were added and incubated for one hour at room temperature. The plate was washed 3× with PBST buffer and the 90Y was measured using a Wallac Microbeta scintillation counter.

### Example 32

### Preparation of Lipid-Based Particles Containing Paclitaxel

The following procedure can be used to prepare vesicles containing 1-10 weight percent paclitaxel. Weigh out 93.4 mg of BisT-PC 13, 6.6 mg of chelator lipid 15, and 1 mg paclitaxel (Sigma). Place in a round bottom flask and add 5 ml chloroform. Swirl to dissolve lipids and paclitaxel. Attach the round bottom to a rotary evaporator equipped with a dry ice/acetone cold trap and lower the flask into a 48° C. water bath. Pull a vacuum while the flask is rotating to remove the chloroform and continue the vacuum for one hour. Remove the flask from the rotary evaporator and add 10 ml of 50 mM HEPES, pH 7.4. Immediately return to the rotary evaporator and rotate in the water bath to resuspend the lipids. Set up a Lipex 10 ml extruder, including a thermobarrel attached to a 48° C. water bath. Place a filter disk (Poretics) in the extruder, followed by a 100 nm polycarbonate track-etched filter (Osmonics), followed by a filter disk and another 100 nm filter. Pipet the lipid mixture into the extruder barrel and wait 5 minutes to equilibrate to the extruder temperature. Extrude the mixture by applying 700 psi of pressure with compressed air. Repeat this process three times, then replace the top filter with a 30 nm filter (Osmonics) and extrude a total of four times. Filter the liposome solution through a 0.2 μm surfactant free cellulose acetate syringe filter and obtain the liposome size by diluting 100 ul into 3 ml 10 mM HEPES, pH 7.4 in a polystyrene cuvette and measuring the size by dynamic light scattering in a Brookhaven ZetaPALS. Quantitation of paclitaxel was obtained by reverse phase HPLC (C18 column, 0.5 ml/min 70% methanol/30% water, 227 nm detection). HPLC analysis also showed that this process did not result in the degradation of paclitaxel.

### Example 33

### Preparation of Lipid-Based Particles Containing Tyrosine Kinase Inhibitors

The following procedure was used to prepare 100 mg of the composition 95% mol/mol BisT-PC 13, 5% mol/mol chelator lipid 15, and 10% w/w AG1433 (2-(3,4-dihydroxyphenyl)-6,7-dimethylquinoxaline, Kroll and Waltenberger, 1997, J. Biol. Chem. 272, 32521; Strawn et al., 1996 Cancer Res. 56, 3540) or SU1498 ((E)-3-(3,5-diisopropyl-4-hydroxyphenyl)-2-[(3-phenyl-N-propyl)amino-carbonyl]acrylonitrile, Strawn et al., 1996 Cancer Res. 56, 3540). Weigh out 93.4 mg of BisT-PC (Avanti), 6.6 mg chelator lipid 15 and 10 mg AG1433 or SU1498 (Calbiochem). Place in a round bottom flask and add 1 ml chloroform and 0.5 ml methanol. Swirl to dissolve lipids and small molecule. Attach the round bottom to a rotary evaporator equipped with a dry ice/acetone cold trap and lower the flask into a 65° C. water bath. Pull a vacuum while the flask is rotating to remove the chloroform and continue the vacuum for one hour. Remove the flask from the rotary evaporator and add 4 ml of 50 mM HEPES, pH 7.4 to resuspend the lipids. Set up a Lipex 10 ml extruder, including a thermobarrel attached to a 90° C. water bath. Place a filter disk (Poretics) in the extruder, followed by a 50 nm polycarbonate track-etched filter (Osmonics), followed by a filter disk and another 50 nm filter. Pipet the lipid mixture into the extruder barrel and extrude the mixture by applying 700 psi of pressure with compressed air. Filter the liposome solution through a 0.2 μm surfactant free cellulose acetate syringe filter and obtain the liposome size by diluting 100 ul into 3 ml 10 mM HEPES, pH 7.4 in a polystyrene cuvette and measuring in a Brookhaven ZetaPALS. Quantitation of the inhibitors was achieved by reverse phase HPLC (C18 column, 0.5 ml/min 70% methanol/30% water, 227 nm detection).

### Example 34

### Preparation of Lipid-Based, Integrin-Targeted Particles Containing Tyrosine Kinase Inhibitors

Integrin-targeted particles containing AG1433 and integrin-targeting lipid 12 are made as described in EXAMPLE 33. For example, the preparation of 100 mg of vesicles containing BisT-PC 13, chelator lipid 15, integrin-targeting lipid 12, and 12.7 weight percent AG1433 was achieved with 90.7 mg BisT-PC, 6.5 mg chelator lipid 4, 2.8 mg integrin-targeting lipid 12, and 12.7 mg AG1433.

### Example 35

### Treatment of a the K1735-M2 Mouse Melanoma Model

The K1735-M2 mouse melanoma model was prepared by subcutaneous injection of tumor cells as previously described (X. Li, et al. Invasion Metastasis 1998, 18, 1-14). Animals received a single i.v. injection of placebo or therapeutic agent and tumor volume was measured until the tumors had quadrupled in size. Tumors were induced in the mice as follows: tumors were implanted by subcutaneous injection of approximately 1×10⁶ K1735 M2 melanoma cells (X. Li, B. Chen, S. D. Blystone, K. P. McHugh, F. P. Ross, D. M. Ramos, Differential expression of alpha v integrins in K1735 melanoma cells. Invasion Metastasis 18(1) (1998) 1-14). The K1735 M2 tumor cells were grown in tissue culture flasks in Dubelco's medium with 10% fetal calf serum (FCS). Cells were harvested using Trypsin-EDTA solution (containing 0.05% trypsin), resuspended in PBS at 10,000,000/ml, and kept on ice. Animals with tumors between 100 and 200 mm³ were selected for treatment.

TABLE 1
Description of treatment groups

| | # | RGD PM | PV Dose | Doxorubicin |
|---|---|---|---|---|
| Group | Animals | Dose (μg/g) | (mg/g) | Dose (μg/g) |
| Buffer | 7 | NA | NA | NA |
| dox10 | 7 | NA | NA | 10 |
| Ldox10 | 7 | NA | 0.1 | 10 |
| ITL | 7 | 1.4 | 0.1 | NA |
| ITL | 7 | 1.4 | 0.1 | NA |
| ITLdox1 | 7 | 1.4 | 0.1 | 1 |
| ITLdox10 | 7 | 1.4 | 0.1 | 10 |

### Example 36

### In vitro Cell Toxicity Measured in a Cell Proliferation Assay

Targeted drug delivery was assessed in vitro by incubating vesicles with MS 1 mouse endothelial pancreatic islet cells and the K1735-M2 murine melanoma tumor cells. The effect of free doxorubicin or liposome-encapsulated doxorubicin on the cells was assayed calorimetrically by crystal violet staining method with slight modification. Mouse endothelial cells (ATCC# CRL-2279) and mouse melanoma cells (M2) were seeded in 96-well flat-bottomed microtitre plates. The effect of the vesicles on cell proliferation was determined with cells near confluence (about 80%). The medium in each well was replaced with 100 ul of culture medium containing 250 ug/ml of vesicles containing doxorubicin. The mouse endothelial and melanoma cells were incubated for 1 hour at 37° C. and 5% CO₂. After 1 hour incubation, the drug was removed, and compete medium that was lacking drug was added, and the cells were incubated for 48 hours at 37° C. and 5% CO₂. The experiment was performed in duplicate. At the end of the incubation period, the cells were washed once with PBS and the cultures were fixed by 70% ethanol overnight. Next, the cells were stained with 100 ul of 0.1% crystal violet in 10% ethanol for 10 minutes at room temperature, and the cells were gently washed with water for 5 times. Next, 100 ul of 1% SDS was added to each well and the plates were placed on an orbital shaker for 15 minutes. After the crystal violet in cell membrane was removed with SDS, the plate was read at 590 nm. Finally, cell viability was evaluated as the mean value of optical density. Results are shown in FIG. 21.

### Example 37

### Preparation of RGD Peptidomimetic Vesicles Containing Paclitaxel DPPC, and Cholesterol

Vesicles identical to those in EXAMPLE 21 were prepared, except the components were DPPC (31.4 mg, 42.8 umole), N-succinyl-DPPE (5 mg, 6.1 umol; Avanti), cholesterol (11.1 mg, 28.6 umol), and paclitaxel (2.5 mg, 2.9 μmole; Sigma). The size measured by dynamic light scattering was 91.1 nm.

### Example 38

### Inhibition of Fibronectin Binding to the αᵥβ₃ Integrin in vitro

96-well plates were coated with 0.1 μg of αᵥβ₃ integrin per well overnight at 4° C. The liquid was removed, and the plates were blocked with 100 μl of Buffer A (25 mM tris, 150 mM NaCl pH 7.2) containing 2% BSA for 2 h, and washed with this buffer three times. RGD peptidomimetic-vesicle conjugates and RGD peptidomimetic-vesicle conjugates containing a therapeutic agent were added in Buffer A containing 0.1% BSA and 1 mM MnCl2 followed by the addition of a fibronectin-HRP conjugate (HRP=horse radish peroxidase). After a 1 h incubation, the plates were washed three times, and 100 ul of chemiluminescent HRP substrate was added. Chemiluminescence was monitored using a Wallac Victor reader. Results are shown in FIG. 22.

### Example 39

### 10,12-tricosadiynoic Acid, NHS Ester (Compound 40)

In a 500 ml round bottom flask were placed 10,12-tricosadiynoic acid (TA, Lancaster, 25 g, 72.2 mmol), N-hydroxysuccinimide (NHS, Aldrich, 13.5 g, 117.2 mmol) and 1-(3-dimethylaminopropyl)-3-ethylcarbodiimide hydrochloride (EDC, Aldrich, 16.2 g, 84.4 mmol). Dichloromethane (580 ml) was added and the mixture was stirred at room temperature, shielded from light for 20 h. The resulting reddish solution was then washed with water/dichloromethane (125 ml each), saturated sodium bicarbonate (125 mL), brine (125 mL), and water (125 mL). The dichloromethane solution was dried over magnesium sulfate, and concentrated to give a slightly bluish solid (30 g). Proton, and carbon NMR were consistent with the desired compound.

### Example 40

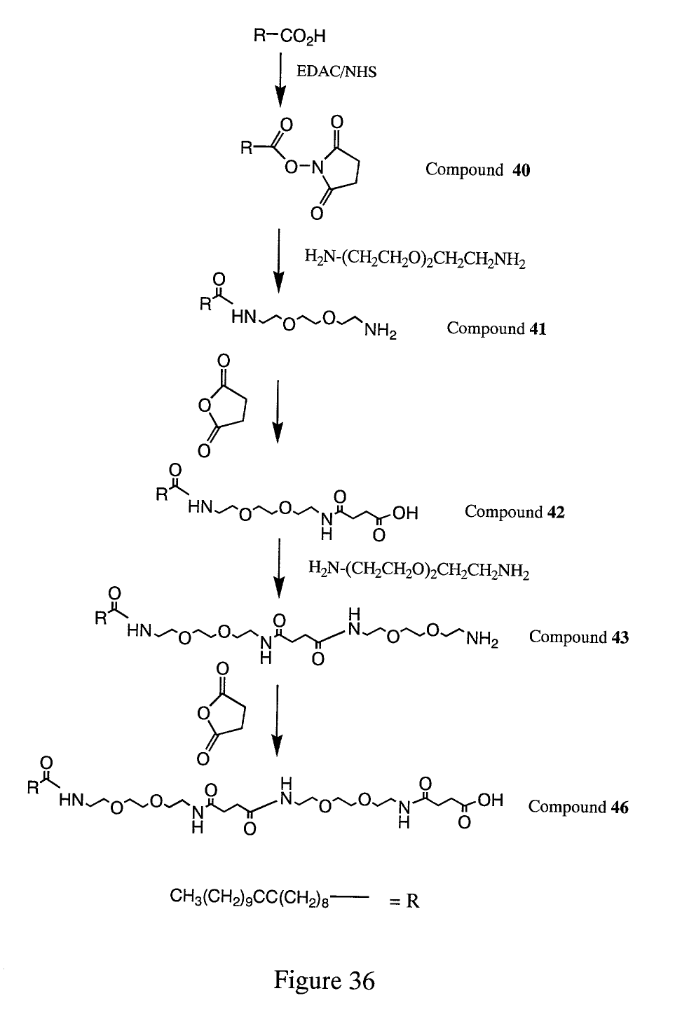

### N-(8′-amino-3′,6′-dioxaoctyl)-10,12-tricosadiynamide (TA-PEG3 Amine) (FIG. 36, Compound 41)

In a 500 ml three-neck flask with a magnetic stirrer was added 10,12-tricosadiynoic acid, NHS ester from EXAMPLE 39 (30 g) in dichloromethane was added from a dropping funnel, slowly, to a stirred solution of 1,8-diamino-3,6-dioxaoctane (PEG3, Jeffamine, Texaco Chemical Co, 28 g, 187 mmol) in dichloromethane (100 mL). The mixture was stirred at room temparature, shielded from light, for 40 h. The resulting emulsion was chromatographed on a silica gel column (9 cm×18 cm) using a gradient of dichloromethane/methanol (25/1 to 8/1). The homogeneous fractions were pooled and concentrated to give 4.8 g of a bluish solid. Proton and carbon NMR, and mass spectrum analysis were consistent with the desired compound.

### Example 41

### N-succinamido-PEG3-TA (SP-TA) (FIG. 36, Compound 42)

TA-PEG3 amine from EXAMPLE 40 (4.79 g, 9.9 mmol) were dissolved in pyridine (50 mL). Some insoluble material was removed by filtration. The solution was concentrated to 25 ml, and succinic anhydride (0.99 g, 9.9 mmol, Aldrich) was added. The mixture was stirred overnight at room temperature, shielded from light. The solution was filtered and concentrated under reduced pressure, followed by evaporation to dryness with methanol (50 mL) twice. The residue was dissolved in acetone (200 ml), and the mixture precipitated upon storage at 4° C. overnight. A reddish powder was isolated by filtration (3.2 g), and the product was recrystallized from methanol (100 ml) to give 2.9 g of a solid. Proton and carbon NMR were consistent with the desired structure.

### Example 42

### N-succinamido-PEG3-TA, NHS ester (NHS-SP-TA) (FIG. 36 Compound 43)

In a 100 ml amber colored bottle with a magnetic stir bar were placed N-succinamido-PEG3-TA from EXAMPLE 41 (1.6 g, 2.77 mmol), N-hydroxysuccinimide (Aldrich, 0.53 g, 4.7 mmol), and EDC (0.7 g, 3.4 mmol). The mixture in dichloromethane (50 ml) was stirred at room temperature, shielded from light for 15 h. The resulting clear solution was washed with water (40 ml). The organic layer was washed with 1% HCl (50 mL), saturated sodium bicarbonate (50 ml), and brine (50 ml). The dichloromethane extracts were dried over anhydrous magnesium sulfate and concentrated under reduced pressure to give 1.6 g of a colorless solid. Proton and carbon NMR are consistent with the desired structure.

### Example 43

### PEG3-succinamido-PEG3-TA (FIG. 36, Compound 44)

In a 100 ml round bottom flask with a magnetic stir bar was placed N-succinamido-PEG3-TA, NHS ester (1.6 g, 2.3 mmol) from EXAMPLE 42 in dichloromethane (25 ml) was added dropwise to a stirred solution of 1,8-diamino-3,6-dioxaoctane (PEG3, Jeffamine, Texaco Chemical Company, 1.1 g, 7.3 mmol) in dichloromethane (5 mL). The mixture was stirred at room temparature, shielded from light, for 40 h. The mixture was concentrated by rotory evaporation, and the residue was dissolved in a small volume of dichloromethane for chromatography on a 4×25 cm silica gel column using a gradient of dichloromethane/methanol (25/1 to 8/1). The appropriate fractions were pooled and concentrated to give 153 mg of colorless solid. Proton and carbon NMR were consistent with the desired compound.

### Example 44

### Preparation of Ligand-Vesicle Conjugates Capable of Modulating Protein Activity

A. Coupling of a protease inhibitor to polymer-coated vesicles: Succinylated aminodextran-coated vesicles (120 mg in 0.414 ml water), water (0.546 ml) MOPS buffer (120 μl of 500 mM) and Ac-LVK-aldehyde (120 μl of 25 mM; Bachem) were added to a 2 ml polypropylene tube with cap and the solution was mixed. To 1 ml of solution, EDAC (0.96 mg, 10 μl) was added. The solution was mixed and incubated at room temperature for 18 hr. The conjugate was dialyzed twice in a 10K MWCO cassette in 3.5 L of 50 mM Histidine buffer containing 5 mM citrate at pH 7.4. Analysis of the conjugate mixture by size exclusion chromatography showed that the coupling yield was approximately 82%

B. Coupling of a protease inhibitor to polymerized vesicles: Polymerized vesicles containing lipid 15 (120 mg in 0.6 ml water), water (0.36 ml) MOPS buffer (120 μL of 500 mM; Sigma) and Ac-LVK-aldehyde (120 μl of 25 mM; Bachem) were added to a 2 ml polypropylene tube with cap and the solution was mixed. To 1 ml of solution, EDAC (0.96 mg, 10 μl) was added. The solution was mixed and incubated at room temperature for 18 hr. Analysis of the conjugate mixture by size exclusion chromatography showed that the coupling yield was approximately 45%. The conjugate was dialyzed twice in a 10K MWCO cassette in 3.5 L of 50 mM Histidine buffer containing 5 mM citrate at pH 7.4.

C. Coupling of a protease inhibitor to polymerized vesicles: GFG-aldehyde semicarbazone was attached to vesicles in the same manner as Ac-LVK-aldehyde.

### Example 45

### Inhibition of Protease Activity

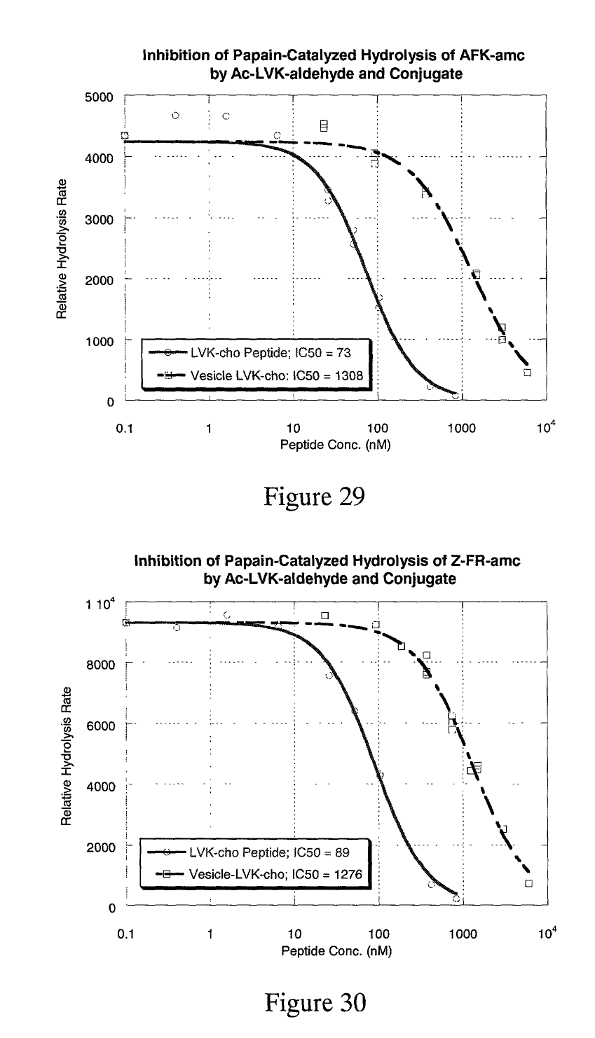

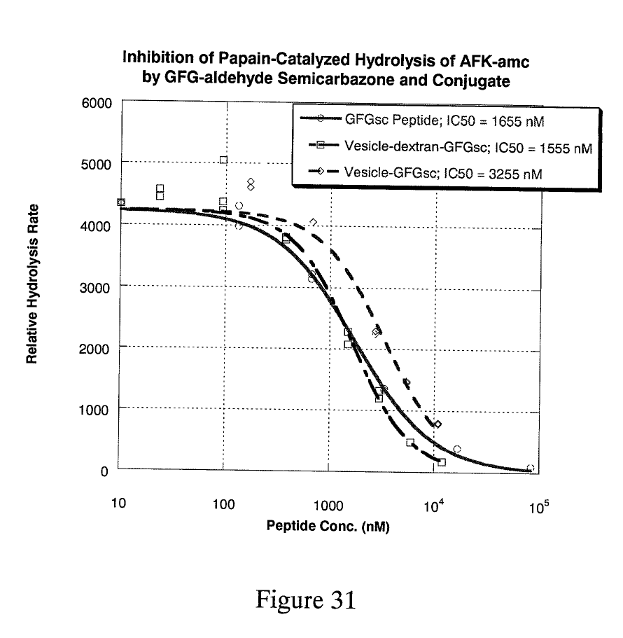

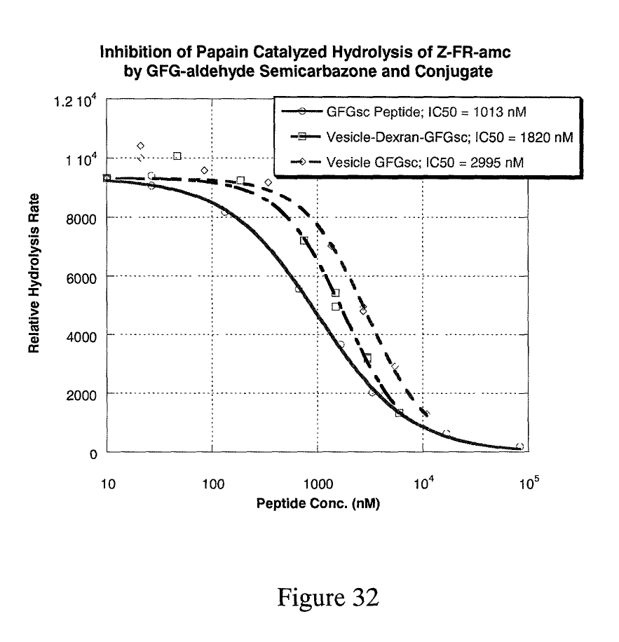

A. Papain Activity Assay. Add substrate (20 ul of 3 mM AFK-7AMC or 2 mM Z-FR-AMC; Bachem) to 3 ml of buffer (50 mM potassium phosphate/1 mM EDTA.5%DMSO pH 6.8) in a 4.5 ml methyl acrylate cuvette (VWR). Add peptide (10 μl of 25 mM GFGsc or 0.25 mM LVK-ald) or inhibitor-vesicle conjugate (20 μl of 0 to 320 μg/ml dilutions in water) to cuvette. Add papain (20 μl of 2 μM Papain in 50 mM potassium phosphate/1 mM EDTA.5%DMSO pH 6.8 containing 5 mM DTT). Cover the cuvette with Parafilm (VWR) and mix by inversion. The cuvette was read immediately on a fluorometer (exc. 380 nm, em. 460 nm; readings at 1-60 sec, Photon Technology). Results are shown in FIGS. 29, 30, 31, and 32.

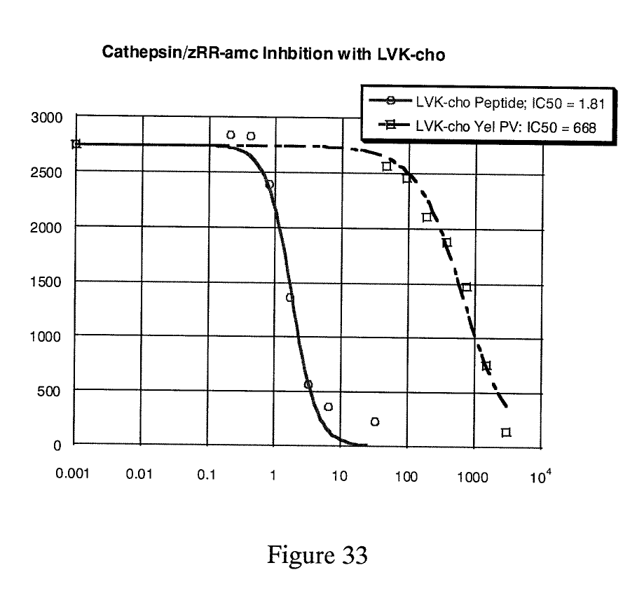

B. Cathepsin Activity Assay. Add cathepsin (15 μl of 1 μM in 50 mM Acetate buffer at pH 5.5 and 5 mM DTT) to 15 μl of peptide inhibitor (2 μM Ac-LVK-cho) or peptide-vesicle conjugate (0 to 80 μg/ml dilutions in water) in a 1.5 ml polypropylene tube and incubate at room temperature for 15 min. Add substrate (10 μl of 4 mM Z-RR-amc; Bachem) to 3 ml buffer (50 mM Acetate buffer at pH 5.5) in 4.5 ml cuvette immediately before adding inhibitor soulution. Add cathepsin inhibitor solutions from Example 45 or unmodified inhibitor (20 μl) to cuvette. Cover the cuvette with Parafilm (VWR) and mix by inversion. The cuvette was read immediately on fluorometer (exc. 380 nm, em. 460 nm; readings at 1-60 sec, Photon Technology). Results are shown in FIG. 33.

### Example 46

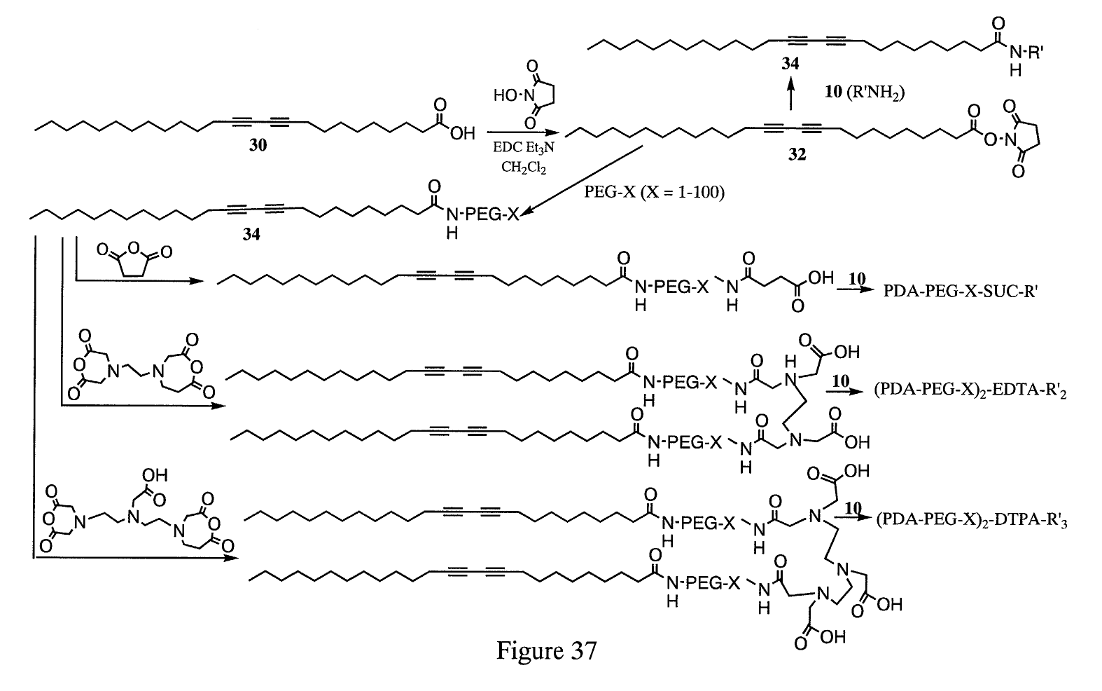

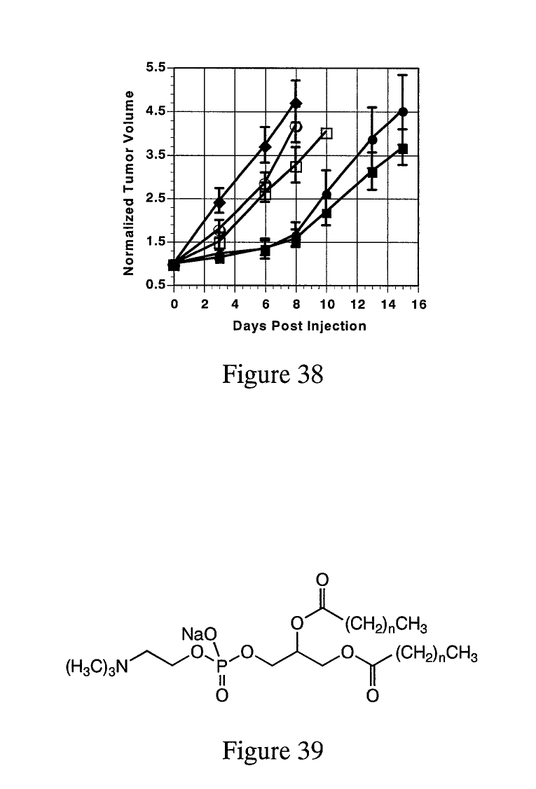

### Preparation of 10,12-Pentacosadiynoic acid N-hydroxysuccinimide ester (PDA-CONHS 32) (FIG. 37)

10,12-Pentacosadiynoic acid (PDA 30) (Lancaster, FW: 374.61, 374 mg, 1 mmole) was dissolved in methylene chloride (Aldrich, 10 mL)) (under argon). To this solution was added N-hyrdroxy succinimide (NHS) (Aldrich, FW: 115.09, 173 mg, 1.5 mmole), and triethylamine (Et₃N) (Aldrich, FW: 101.19, d: 0.726, 0.4 mL 3 mmole). The solution was stirred to dissolve. EDC was added (Aldrich, FW: 191.71, 288 mg. 1.5 mmole) and stirred at room temperature overnight. TLC of the reaction mixture showed complete disappearance of the starting material and a single product. The reaction mixture was diluted with methylene chloride (100 mL), washed with 0.1 N HCl (25 mL), water (25 mL), and finally with brine (25 mL). The organic layer was dried over anhydrous sodium sulfate and the solvent then removed by spin evaporation. The crude product thus obtained (401 mg, 85% yield) was used without further purification.

### Example 47

### EXAMPLE Preparation of 10,12-Pentacosadiynoic polyethyleneglycolamide (PDA-CONH-PEG33 36) (FIG. 37)

PDA-CONHS from EXAMPLE 46 (401 mg, 0.85 mmole) was dissolved in methylene chloride (Aldrich, 10 mL)under argon. To this solution was added PEG33 (Huntsman, FW: 2000, 2.55 g, 1.28 mmole) using a syringe pump during a period of 5 h. TLC showed complete disappearance of the starting material. The reaction was then diluted with methylene chloride (200 mL) and washed with 0.1N HCl (3×50 mL). The organic layer was then washed with water (50 mL) and finally with brine (50 mL), and dried over anhydrous sodium sulfate. The residue was partitioned between water (250 mL) and CH₂Cl₂ (25 mL) to remove remaining PEG33.

The CH₂Cl₂ layer was separated, dried over anhydrous sodium sulfate, filtered to remove the sodium sulfate, and the solvent removed by spin evaporation. The product 36 was dried under high vacuum.

### Example 48

### Preparation of 4-[2-(3,4,5,6-Tetrahydropyrimidin-2-ylamino)ethyloxy]benzoyl-2-(S)-(10′,11′-Pentacosadiynoic amidoethylsulfonylamino)-β-alanine (PDA-PM 34) (FIG. 37)

PDA-CONHS from EXAMPLE 46 (141.3 mg, 300 μmol) and compound 10 (FIG. 16) (SRI, FW: 456, 162.5 mg, 330 μmol) were dissolved in anhydrous pyridine (Aldrich, 10 mL) in a previously flame dried flask filled with argon. The solution was stirred overnight for two nights. The reaction was then stirred under reflux. After 6 h the starting material was still present. To the reaction was added another 163 mg of PM (330 μmol). The reaction was stirred under reflux over night. The reaction mixture was spin evaporated under high vacuum to remove the solvent. The product mixture was dried under high vacuum overnight. Yield of the crude product was 125.7 mg (51.6%, 1-00% =244 mg). The product 34 was used without further purification.

### Example 49

### Preparation of Vesicles Containing 10% PDA-PM, 10% PDA-DTPA 1% PDA-DTPA-Eu, and 79% PC (FIG. 37).

| | | | mol % of | | Amt. Of |
|---|---|---|---|---|---|
| Material | MW | Supplier | lipid | [lipid] | Lipid |
| PDA-PM | 812 | | 10% | 3 mM | 9.7 mg |
| PDA-DTPA-Eu | 1517 | | 1% | 0.3 mM | 182 μg* |
| PDA-DTPA | 1367 | SRI | 10% | 3 mM | 16.4 mg |
| PC | 914 | Avand | 79% | 23.7 mM | 86.9 mg |

*182 μL of a Img/mL solution in CHCl₃

The above lipids (114.82 mg, 4 mL) were mixed in test tubes, dissolved in 0.5 mL of CHCl₃, spin evaporated to dryness, and dried under high vacuum overnight. The effective concentration of PM (compound 10) was 3 mM (1.37/mg/mL)

The dried residue was suspended in 4 mL of water and sonicated for about one hour while checking the pH frequently.

The formed vesicles were polymerized by first cooling the solution in a petri dish in an ice bath and then placing under an UV lamp for 120 min. The solution was then dialyzed in 50 mM histidine, 5 mM sodium citrate, pH 7.4 overnight.

Physical parameters of the polymerized vesicles (PVs) were as follows: 47.7 nm effective diameter, 53.3 nm mean diameter, zeta potential, −94.55 mv, λₘₐₓ=486 nm and 518 nm, pH=7.4

The solution was removed from the dialysis cassette using a 30 mL naked syringe. The needle was removed from the syringe and was fitted with a 0.2μ filter and the particles were filtered into a vial. Size and zeta potential were measured by diluting 25 μL of the PV with 2 mL of water.

What is claimed is:

1. A targeted therapeutic agent, comprising a linking carrier, a therapeutic entity associated with the linking carrier, and at least one targeting entity.

2. The targeted therapeutic agent of claim 1, wherein the linking carrier comprises an amount of targeting entities selected from the group consisting of two or more targeting entities, ten or more targeting entities, 100 or more targeting entities, and 1000 or more targeting entities.

3. The targeted therapeutic agent of claim 1, wherein the targeting entity is present at a concentration from 0.1 to 30 mole percent.

4. The targeted therapeutic agent of claim 1, wherein the linking carrier is selected from the group consisting of a liposome, and a polymerized vesicle.

5. The targeted therapeutic agent of claim 4, wherein said linking carrier comprises a phosphatidylcholine derivative.

6. The targeted therapeutic agent of claim 1, wherein said targeting entity targets the lipid construct to a target selected from the group consisting of a cell surface target, an intracellular target, and an extracellular matrix component.

7. The targeted therapeutic agent of claim 1, wherein the targeting entity is associated with the lipid construct by covalent means.

8. The targeted therapeutic agent of claim 1, wherein the targeting entity is associated with the lipid construct by non-covalent means.

9. The targeted therapeutic agent of claim 1, wherein said targeting entity has a vascular target.

10. The targeted therapeutic agent of claim 1, wherein said targeting entity has a vascular target selected from the group consisting of chemokine receptors CCR4 and CCR5, VCAM, FGFR, matrix metallopreases (MMPs) including surface associated MMPs, PDGFR, P- and E-selectins, pleiotropin, Flk-1/KDR. Flt-1, Tek, Tie, neuropilin-1, endoglin, endosialin, Ax1, the integrins including αᵥβ₃, αᵥβ₅, α₅β₁, α₄β₁, α₁β₁, α₂β₂, or prostate specific membrane antigen (PSMA).

11. The targeted therapeutic agent of claim 1, wherein said targeting entity has a tumor cell target.

12. The targeted therapeutic agent of claim 1, wherein said targeting entity has a tumor cell target selected from the group consisting of chemokine receptors CCR4 and CCR5, VCAM, EGFR, FGFR, matrix metalloproteases (MMPs) including surface associated MMPs, PDGFR, P- and E-selectins, pleiotropin, Flk-1/KDR, Flt-1, Tek, Tie, neuropilin-1, endoglin, endosialin, Ax1, the integrins including αᵥβ₃, αᵥβ₅, α₅β₁, α₄β₁, α₁β₁, α₂β₂, or prostate specific membrane antigen (PSMA).

13. The targeted therapeutic agent of claim 1, wherein the targeting entity is an integrin-specific molecule.

14. The targeted therapeutic agent of claim 13, wherein the integrin-specific molecule comprises an RGD peptide.

15. The targeted therapeutic agent of claim 13, wherein the integrin-specific molecule comprises 3-{4-[2-(3,4,5,6-tetrahydropyrimidin-2-ylamino)-ethyloxy]-benzoylamino}-2(S)-benzene-sulfonyl-aminopropionic acid.

16. The targeted therapeutic agent of claim 13, comprising a compound of the formula: 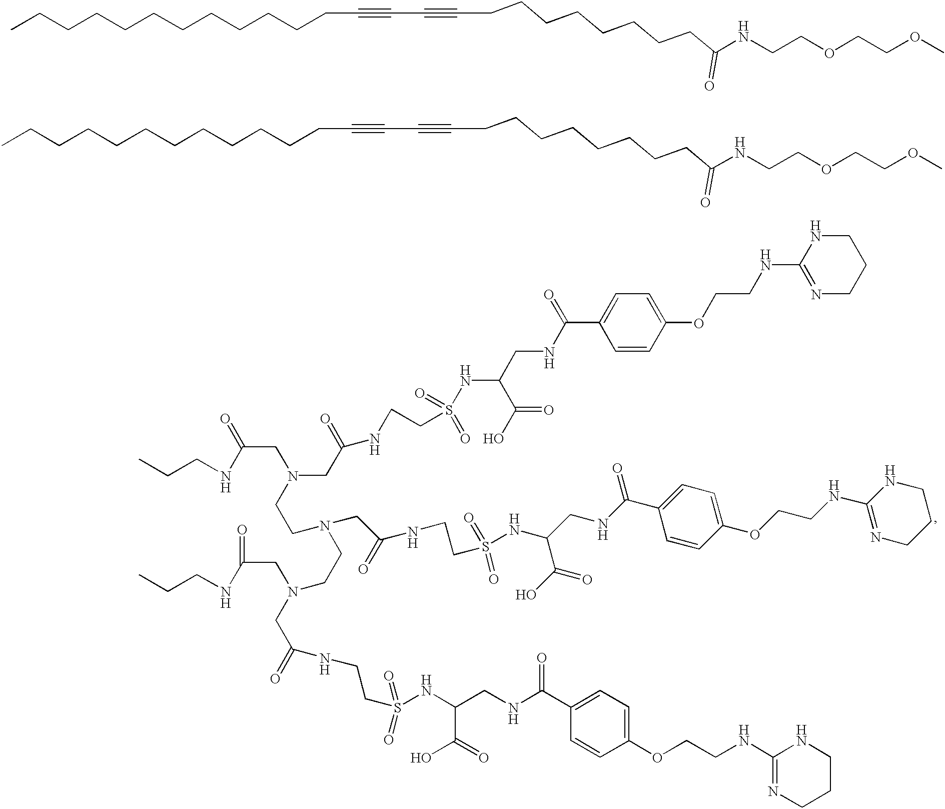
wherein the compound is associated with the lipid construct by non-covalent or covalent means.

17. The targeted therapeutic agent of claim 1, wherein the targeting entity is a kinase modulator.

18. The therapeutic agent of claim 17, wherein the kinase modulator is AG1433 or SU1498.

19. The targeted macromolecule of claim 1, wherein the targeting entity is a protease-specific molecule.

20. The therapeutic agent of claim 19 wherein the protease-specific molecule is a peptide or peptidomimetic having a C-terminal aldehyde or derivative thereof.

21. The targeted therapeutic agent of claim 1, further comprising a stabilizing entity.

22. The targeted therapeutic agent of claim 21, wherein the stabilizing entity is selected from the group consisting of a natural polymer, a semi-synthetic polymer, and a synthetic polymer.

23. The targeted therapeutic agent of claim 22, wherein the stabilizing entity is selected from the group consisting of dextran, modified dextran, and poly(ethylene imine).

24. The targeted therapeutic agent of claim 21, wherein the stabilizing entity provides the capacity for multivalency.

25. The targeted therapeutic agent of claim 1, wherein the therapeutic entity is present at a concentration of about 1% to about 40%.

26. The targeted therapeutic agent of claim 1, wherein the therapeutic entity is selected from the group consisting of doxorubicin, daunorubicin, epirubin, and idarubicin.

27. The targeted therapeutic agent of claim 26, wherein the therapeutic entity is doxorubicin.

28. The targeted therapeutic agent of claim 1, wherein the therapeutic entity is a taxane compound.

29. The targeted therapeutic agent of claim 28, wherein the taxane compound is paclitaxel or docetaxel.

30. The targeted therapeutic agent of claim 1, wherein the therapeutic entity is camptothecin or topotecan.

31. The targeted therapeutic agent of claim 1, wherein the therapeutic entity is selected from the group consisting of matrix metalloprotease inhibitors, analgesics, aggrecanase inhibitors, osteoclast inhibitors, alkylating agents, cisplatinum and derivatives, pyrimidine and purine analogues, topoisomerase inhibitors, microtuble-targeting agents, estrogen derivatives, androgen derivatives, interferons, intercalating agents, kinase inhibitors, and MDR inhibitors.

32. The targeted therapeutic agent of claim 1, wherein the therapeutic entity is doxorubicin and the targeting entity is 3-{4-[2-(3,4,5,6-tetrahydropyrimidin-2-ylamino)-ethyloxy]-benzoylamino}-2(S)-benzene-sulfonyl-aminopropionic acid.

33. A method of preparing a targeted therapeutic agent, comprising providing a targeted lipid construct, said targeted lipid construct comprising more than one targeting entity, and associating a therapeutic entity within the lipid construct.

34. The method of claim 33, wherein targeted lipid construct comprises a lipid construct selected from the group consisting of liposomes, micelles, vesicles, and polymerized liposomes.

35. The method of claim 33, wherein the therapeutic entity is selected from the group consisting of doxorubicin, daunorubicin, epirubin, and idarubicin.

36. The method of claim 34, wherein the therapeutic entity is a taxane compound.

37. The method of claim 35, wherein the taxane compound is paclitaxel or docetaxel.

38. The method of claim 36, wherein the therapeutic entity is camptothecin or topotecan.

39. A method of treating a patient in need thereof comprising administering an effective amount of a pharmaceutical composition comprising a linking carrier, said linking carrier comprising at least one targeting entity, and an associated therapeutic entity to a patient need thereof.
